(19) United States
(12) Patent Application Publication
(10) Pub. No.: US 2010/0330013 A1
(43) Pub. Date: Dec. 30, 2010

(54) AMADORIASES IN WASHING AND CLEANING PRODUCTS

(75) Inventors: Timothy O'Connell, Duesseldorf (DE); Nina Hoven, Duesseldorf (DE); Petra Siegert, Haan (DE); Karl-Heinz Maurer, Erkrath (DE)

Correspondence Address: Ratner Prestia, P.O. Box 980, Valley Forge, PA, 19482, US

(73) Assignee: HENKEL AG & CO. KGaA, Duesseldorf (DE)

(21) Appl. No.: 12/266,860

(22) Filed: Nov. 7, 2008

Related U.S. Application Data
(63) Continuation of application No. PCT/EP2007/003997, filed on May 7, 2007.

(30) Foreign Application Priority Data
May 8, 2006 (DE) 10 2006 021 401.3

(51) Int. Cl. A61K 8/66; C12N 9/06; A61K 38/44; A61K 38/54; A61Q 5/08; A61Q 5/02; A61Q 19/02; A61Q 19/10; C11D 3/395

(52) U.S. Cl. 424/62; 435/191; 424/94.4; 424/94.2; 424/70.1; 252/186.1; 435/264

(57) ABSTRACT

Body care products, hair care products, shampoos, oral care, dental care and dental prosthesis care products, dental brace care products, cosmetics, therapeutics, washing agents, cleaning agents, brightening products, disinfectants, rinsing agents, hand washing products, dishwashing agents, machine dishwashing agents and agents for the bleaching and disinfecting treatment of filter media, textiles, furs, paper, skins or leather, and other products containing amadoriases are provided. Also, the use of amadoriases for decomposition products of the Amadori rearrangement is provided.

### CROSS REFERENCE TO RELATED APPLICATIONS

This application is a continuation of PCT Application Serial No. PCT/EP2007/003997, filed May 7, 2007, and claims priority to German Patent Application Serial No. 102006021401.3, filed May 8, 2006. These applications are incorporated by reference herein, in their entirety and for all purposes.

### FIELD

The present invention relates to body care products, hair care products, hair shampoos, compositions for oral care, dental care and dental prosthesis care, dental brace care products, cosmetics, therapeutics, laundry detergents, cleaning compositions, brightening agents, bleaching agents, disinfectants, rinse agents, detergents for hand washing, dish washing detergents, automatic dishwasher detergents and compositions for bleaching or disinfecting filter media, textiles, furs, paper, hides or leather, which comprise amadoriases as well as uses of amadoriases.

### BACKGROUND

Stains of biological origin, especially those originating from foodstuffs, are particularly intractable when heated. Complex reactions proceed on heating, which are summarized by the term “Maillard Reaction” or also “Non-enzymatic browning”. The reducing sugars comprised in the foodstuffs react with an amino group of the proteins, peptides or amino acids. This yields the derivative of an N-glycosylamine, from which the derivative of a 1-amino-desoxy-2-ketose “amino ketose” is formed by the Amadori rearrangement. Finally, there occurs a great number of further reactions that lead to e.g. melanoids (browning), various aromas and crosslinked proteins.

These Amadori products or products of the Maillard reaction are either already comprised in the stain on the fabric or tableware or first develop during the cleaning and thereby possibly accentuate the discoloration. An effective and gentle composition for removing such discolorations is not known in the prior art.

Consequently there exists a not insubstantial need for compositions that counteract the unwanted discoloration caused by these Amadori products.

### BRIEF DESCRIPTION OF THE DRAWINGS

FIG. 1 shows the results of a laundry test comparing the cleaning power of an amadoriase-containing cleaning composition.

FIG. 2 shows the results of a laundry test comparing the cleaning power of an amadoriase-containing cleaning composition, whereby the Bacillus licheniformis protease concentrate was purified.

### DETAILED DESCRIPTION

Accordingly, the subject matter of the present invention is a laundry detergent, cleaning composition, rinse aid, detergent for hand washing or automatic dishwasher detergent, comprising at least one amadoriase.

In the context of the present invention, an amadoriase is defined as an enzyme that is capable of decomposing the products of the Amadori rearrangement.

The amadoriase comprised in the composition according to the invention decomposes the above-described products of the Amadori rearrangement and thereby advantageously prevents the typical, intractable stains that result from the Maillard reaction.

The action of the amadoriase is synergistically reinforced by the addition of a protease that increases the availability of a substrate of the amadoriase, the peptide. The action of the protease is again reinforced by the decomposition of the Amadori products, as these represent reversible inhibitors of the protease. Moreover, the reversible inhibition leads to a stabilization of the enzyme in the composition according to the invention, which is offset by the uptake of the substrate by the amadoriase.

The stains resulting from the Maillard reaction are partly formed first in the washing machine or in the dishwasher. This formation is prevented or reduced by the use of the amadoriase in the composition according to the invention.

In addition, in a particularly preferred embodiment of the composition according to the invention, the use of the amadoriase produces an advantageous bleaching action if the composition comprises at least one surfactant and at least one perhydrolase. A composition that comprises at least one bleach activator and/or at least one bleach catalyst is likewise particularly preferred. The stain and possibly also the substances comprised in the laundry detergent/cleaning composition is or are then oxidized by the amadoriase and in this way hydrogen peroxide is produced. This reacts with the perhydrolase that was also added to yield a peracid and leads to the desired bleaching agent. Compared with the conventional direct addition to the product in the prior art, this enzymatic production of hydrogen peroxide has the advantage inter alia that the substance is first produced directly during the application, storage instability is avoided, loss of the hydrogen peroxide by decomposition during storage is avoided, and that no hydrogen peroxide has to be declared as an ingredient of the formulation. A further advantage is the continuous post-production of hydrogen peroxide, as this is withdrawn by its reactivity from the equilibrium.

This bleaching action can also be utilized in compositions other than the abovementioned inventive compositions, as long as said compositions comprise at least one surfactant and at least one perhydrolase.

Accordingly, further subject matters of the present invention are body care compositions, hair care compositions, hair shampoos, compositions for oral care, dental care and dental prosthesis care, dental brace care products, cosmetics, therapeutics, brightening agents, bleaching agents, disinfectants and compositions for bleaching or disinfecting filter media, textiles, furs, paper, hides or leather, which comprise at least one amadoriase.

According to the invention, an amadoriase that can be extracted from fungi or bacteria is preferred. Amadoriases from the species Aspergillus and Bacillus are particularly preferred.

Particularly preferred amadoriases are those that can be obtained from organisms selected from among Agrobacterium tumefaciens, Agrobacterium tumefaciens, Arthrobacter sp., Aspergillus clavatus, Aspergillus fumigatus, Aspergillus nidulans, Aspergillus oryzae, Aspergillus terreus, Bacillus halodurans, Candida boidinii, Coniochaeta sp., Corynebacterium sp., Cryptococcus neoformans, Debaryomyces hansenii, Eupenicillium terrenum, Filobasidiella neoformans, Gibberella zeae, Neosartorya fischeri, Ulocladium sp., Fusarium oxysporum, Acremonium, Bacillus cereus, Bacillus clausii; Brevibacterium linens, Thermobifidafusca; Bacillus anthracis, Bacillus thuringiensis, Eobacillus kaustophilus, Bacillus subtilis, Oceanobacillus iheyensjs, Geobacillus kaustophilus, Penicillium spp., Penicillium janthinellum, Pichia sp., Bacillus licheniformis and Schizosaccharomyces pombe.

Amadoriases are quite particularly preferred, whose amino acid sequences include a sequence or consist of a sequence, which match the amino acid sequences listed in the SEQ ID NO. 1 to 26, to at least 80, 85 or 90%, preferably to at least 91, 92 or 93%, especially to at least 94, 95 or 96%, particularly preferably to at least 97, 98 or 99% and quite particularly preferably to 100%. Such amadoriases are obtainable for example by one or multiple conservative amino acid exchanges from an amadoriase according to one of the sequences 1 to 26, or by derivatization, fragmentation, deletion mutation or insertion mutation of an amadoriase according to one of the sequences 1 to 26.

In the context of the present application, a protein is understood to mean a polymer composed of natural amino acids, which is essentially linear in structure and which assumes in the main a three dimensional structure for carrying out its function. In the present application, the 19 proteinogenic, naturally occurring L-amino acids as well as glycine are identified with the customary international 1- and 3 letter codes.

In the context of the present application, an enzyme is understood to mean a protein that has a specific biocatalytic function.

In the context of the present application, nucleic acids are understood to mean the molecules that are naturally constructed from nucleotides, which serve as information carriers and code for the linear amino acid sequence in proteins or enzymes. They can be present as a single strand, as a complementary single strand to this single strand or as a double strand. Nucleic acid DNA is preferred as the naturally, long lasting information carrier for molecular biological work. On the other hand, an RNA is formed for the realization of the invention in natural surroundings, such as for example in an expression cell, which is why RNA molecules that are essential for the invention also represent embodiments of the present invention.

In DNA the sequences of both complementary strands have to be taken into account in each of all three possible reading frames. In addition, it has to be taken into account that different codon triplets can code for the same amino acids, with the result that a specific amino acid sequence can be derived from a plurality of different and nucleotide sequences exhibiting possibly only slight identity (degeneracy of the genetic code). Moreover, various organisms exhibit differences in the use of these codons. On these grounds, both amino acid sequences as well as nucleotide sequences have to be included in considerations of the field of protection, and listed nucleotide sequences are only to be regarded as an example of coding for a specific amino acid sequence.

The information unit corresponding to a protein is also designated as a gene in the context of the present application.

The present invention includes the manufacture of recombinant proteins. According to the invention, processes for their manufacture include all gene technical or microbiological processes that are based on the fact that the genes for the proteins of interest are brought in a host organism that is suitable for the production and are transcribed and translated by it. The gene in question is suitably incorporated through vectors, especially expression vectors, but also through those that cause the gene of interest in the host organism to be inserted into an already present genetic element such as the chromosome or another vector. The Functional unit of gene and promoter and possibly additional genetic elements is designated as the expression cassette according to the invention. However, it must not also necessarily be present as a physical unit.

Using today's generally known methods, such as for example chemical synthesis or the polymerase chain reaction (PCR) in combination with molecular biological and/or protein chemical standard methods, it is possible for the person skilled in the art to manufacture, with the help of known DNA sequences and/or amino acid sequences, the corresponding nucleic acids up to complete genes. Such methods are known for example from Sambrook, J., Fritsch, E. F. and Maniatis, T. 2001. Molecular cloning: a laboratory manual, 3rd Edition Cold Spring Laboratory Press.

Modifications of the nucleotide sequence, as can be brought about by known molecular biological methods, are called mutations. Known types depend on the nature of the modification, for example deletion mutations, insertion mutations or substitution mutations or those in which various genes or parts of genes are fused together or recombined; they are gene mutations. The associated organisms are called mutants. The proteins derived from mutated nucleic acids are called variants. Thus, for example deletion-, insertion-, substitution mutations or fusions lead to deletion-, insertion-, substitution mutants or fusion genes and at the protein level to corresponding deletion-, insertion- or substitution variants or fusion proteins.

Fragments are understood to mean all proteins or peptides, which are smaller than natural proteins or those that correspond to completely translated genes, and for example can also be obtained synthetically. Due to their amino acid sequences, they can be assigned to the relevant complete proteins. For example, they can assume the same structure or exercise catalytic activities or partial activities, such as for example the complexation of a substrate. Fragments and deletion variants of starting proteins are in principle very similar; while fragments depict rather smaller debris, the deletion mutants rather lack only short regions, and therefore only a few partial functions.

At the nucleic acid level, the partial sequences correspond to the fragments.

In the context of the present application, chimeric or hybrid proteins are understood to mean those proteins that are coded from nucleic acid chains that naturally come from different or from the same organism. This procedure is also called recombination mutagenesis. The sense of such a recombination can consist in, for example, providing or modifying a specific enzymatic function with the help of the fused-on protein part. It is irrelevant in the context of the present invention whether such a chimeric protein consists of a single polypeptide chain or a plurality of sub-units, onto which various functions can be distributed.

“Proteins obtained by means of insertion mutation” are understood to mean those variants that have been obtained by known methods of inserting a nucleic acid fragment or protein fragment into the starting sequences. Due to their fundamental similarity, they are classified as chimeric proteins. They differ from those only in the proportion of the size of the unchanged part of the protein to the size of the whole protein. In these insertion mutated proteins, the share of foreign protein is less than in chimeric proteins.

Inversion mutagenesis, meaning a partial reversal of the sequence, can be regarded as a special form of both deletion as well as of insertion. The same is true for new groupings of different molecular parts that differ from the original amino acid sequence. It can be regarded both as a deletion variant, as an insertion variant as well as a shuffling variant of the original protein.

In the context of the present application, derivatives are understood to mean proteins, whose particular amino acid chain has been chemically modified. Such derivatizations can be effected biologically, for example by the host organism in connection with the protein biosynthesis. Molecular biological methods can be used for this. However, they can also be effected chemically, for example by the chemical transformation of a side chain of an amino acid or by the covalent bonding of another compound onto the protein. This type of compound can also concern other proteins for example that are bonded to the inventive protein through a bifunctional chemical compound, for example. These types of modification can influence, for example, the substrate specificity or the binding strength to the substrate or provide a temporary blocking of the enzymatic activity in the case where the attached substance is an inhibitor. This can be meaningful for the storage period, for example. Similarly, derivatization is also understood to mean the covalent bonding to a macromolecular support.

Proteins can also be assimilated to groups of immunologically related proteins by reaction with an antiserum of a specific antibody. The members of a group are characterized in that they possess the same antigen determinant recognized by an antibody.

In the context of the present invention, all enzymes, proteins, fragments and derivatives, in so far as they do not need to be explicitly treated as such, are assimilated under the generic term proteins.

In the context of the present invention, vectors are understood to mean elements that consist of nucleic acids, which comprise a gene of interest as the characterizing nucleic acid region. They are able to establish the gene as a stable genetic element in a species or a cell line over several generations or cell divisions. Vectors, particularly when used in bacteria, especially plasmids, are therefore circular genetic elements. In gene technology, a differentiation is made, on the one hand, between those vectors that serve the storage and thereby to a certain extent also the technical genetic work, the so called cloning vectors, and on the other hand, those that fulfil the function of realizing the gene of interest in the host cells, i.e. to enable the expression of the protein in question. These vectors are called expression vectors.

By comparing with known enzymes, which for example have been deposited in generally accessible data banks, the enzymatic activity of an enzyme under study can be deduced from the amino acid sequence or the nucleotide sequence. This can be qualitatively or quantitatively modified by other regions of the protein, which do not participate in the actual reaction. This can concern, for example, the enzyme stability, the activity, the reaction conditions or the substrate specificity.

This comparison is made by assigning similar sequences in the nucleotide sequences or amino acid sequences of the studied protein with one another. This is called homologization. A tabular assignment of the positions is called the alignment. When analyzing nucleotide sequences, both complementary strands and each of all three possible reading frames have again to be taken into account; the same goes for the degeneracy of the genetic code and the organism-specific codon usage. Alignments have since been drawn up by means of computer programs, such as, for example by the algorithms FASTA or BLAST; this method is described, for example, by D. J. Lipman and W. R. Pearson (1985) in Science, volume 227, pp. 1435-1441.

A compilation of all matching positions in the compared sequences is called a consensus sequence.

A comparison of this type allows a statement to be made of the similarity or homology of the compared sequences to one another. This is reported in percent identity, i.e. the proportion of identical nucleotides or amino acid residues in the same positions. Another accepted homology term includes the conservative amino acid exchanges in this value. This is then termed the percent similarity. Such statements can refer to the whole protein or gene or only to specific regions.

The construction of an alignment is the first step for defining a sequence space. This hypothetical space includes all sequences obtained by permutation in single positions, which can occur by considering all variations appearing in the relevant single positions of the alignment. Every hypothetically possible protein molecule forms a point in this sequence. For example, two amino acid sequences that each exhibit two different amino acids at only two different positions in a complete identity, therefore establish a sequence space of four different amino acid sequences. A very large sequence space is obtained when additional homologous sequences are each found for single sequences of a space. For those high homologies consisting in pairs, also very low homologous sequences can be recognized as belonging to a sequence space.

Homologous regions of different proteins are defined by matching the amino acid sequence. They can also be characterized by identical functions. This goes as far as complete identities in the smallest region, so called boxes, which include only a few amino acids and mostly exercise essential functions for the overall activity. Functions of the homologous regions are understood to mean the smallest partial functions of the function exercised by the whole protein, such as for example the formation of single hydrogen bonds for complexing a substrate or transition complex.

In the context of the present invention, the nucleic acid is suitably cloned into a vector. The molecular biological dimension of the invention accordingly consists in vectors with the genes for the corresponding proteins. For example, they can include those that derive from bacterial plasmids, from viruses or from bacteriophages, or essentially synthetic vectors or plasmids with elements from the most different origin. Vectors with each of the additional available genetic elements are able to establish themselves in the relevant host cells for several generations to as far as stable units. Accordingly, in the context of the invention, it is irrelevant whether they establish themselves extrachromosomally as their own units or are integrated into a chromosome. Whichever of the numerous systems known from the prior art is selected, depends on the individual case. The achievable number of copies, the available selection systems, principally among them resistance to antibiotics, or the ability to cultivate host cells that can take up the vectors, for example, can be decisive.

The vectors form suitable starting points for molecular biological and biochemical investigations of the relevant genes or associated proteins and for further developments according to the invention and finally for the amplification and production of proteins according to the invention. In this respect, they illustrate embodiments of the present invention, as the sequences of the resulting inventively employable nucleic acid regions each lie within the homology regions more precisely designated above.

Besides storage, biological amplification or selection of genes of interest for the characterization of the relevant genes, cloning vectors are suitable, for example, for building a restriction map or the sequencing.

Expression vectors are chemically similar to cloning vectors, but differ in each partial sequence that enables them to replicate host organisms optimized for the production of proteins and to bring the resulting gene to expression there. The expression is influenced, for example by promoters that regulate the transcription of the genes. Thus, the expression can occur by means of the natural, original, localized promoter with this gene, but also after gene technical fusion, both by means of a prepared promoter of the host cell on the expression vector and also by a modified or a completely other promoter of another organism.

Those expression vectors that are preferred can be regulated by changing the conditions of culture or by adding certain compounds, such as for example the cell density or specific factors. Expression vectors permit the associated protein to be produced heterologously, i.e. in a different organism as that from which it can be naturally obtained. A homologous protein production from a host organism that naturally expresses the gene over an appropriate vector lies within the field of protection of the present invention. This can have the advantage that natural, modification reactions in a context of the translation on the resulting protein can be carried out in the same way as they would normally be.

According to the invention, cell-free expression systems, in which the protein biosynthesis is reconstructed in vitro, can also be employed. Such expression systems are also established in the prior art.

The in vivo synthesis of an enzyme, i.e. by living cells, requires the transfer of the associated gene into a host cell, its so called transformation. In principle, all organisms, i.e. prokaryotes or eukaryotes, are suitable host cells. Those host cells are preferred, which can be genetically handled with ease, for example in relation to the transformation with the expression factor and its stable establishment, for example single cell fungi or bacteria. In addition, preferred host cells are those with a good microbiological and biotechnological handleability. For example this relates to ease of cultivation, high growth rates, low demands on fermentation media and good production rates and secretion rates for foreign proteins. Frequently, the optimum expression system for the individual case must be experimentally determined from the abundance of different systems available from the prior art. Each inventive protein can be obtained in this way from a plurality of host organisms.

Such host cells are preferred that can be regulated in their activity due to the genetic regulation elements that are, for example, made available to the expression vector, but which can also be already present in these cells. For example, they can be stimulated to expression by the controlled addition of chemical compounds that serve as activators, by changing the cultivation conditions or by attaining a specific cell density. This enables a very economical production of the products of interest.

Preferred host cells are prokaryotic or bacterial cells. Bacteria, in comparison with eukaryotes, generally have shorter generation times and lesser demands on the cultivation conditions. This enables cost effective processes for obtaining the interesting proteins to be established. In gram-negative bacteria, such as Escherichia coli (E. coli), a large number of proteins are secreted into the periplasmatic space, i.e. into the compartment between both the membranes that encapsulate the cells. This can be advantageous for specific applications. On the other hand, gram-positive bacteria, such as bacilli or actinomycetes or other representatives of the actinomycetes, possess no external membrane, such that secreted proteins are immediately emitted into the alimentation medium surrounding the cells, from which according to another preferred embodiment the expressed inventively employable proteins can be directly purified.

Expression systems illustrate a variant of this experimental principle, in which additional genes, for example those that are made available on other vectors, influence the production of interesting proteins. They can be modified gene products or those intended to be purified together with the interesting protein, for example to influence its enzymatic function. They can be other proteins or enzymes, for example, inhibitors or such elements that influence the interactions with various substrates.

Due to the far-reaching experience obtained with regard to, for example the molecular biological methods and the cultivation with coliform bacteria, they are preferred for obtaining the inventively employable enzymes. Those of the genera Escherichia coli, especially non-pathogenic strains suitable for the biotechnological production, are particularly preferred.

Representative members of these genera are the K12 derivatives and the B-strains of Escherichia coli. Strains that can be derived from them according to known genetic and/or microbiological methods and thereby can be considered as their derivatives, possess the most important significance for genetic and microbiological work and are preferably employed for the development of inventive processes. Such derivatives can be modified for example through deletion mutagenesis or insertion mutagenesis in regard to their demands on the conditions of culture, exhibit other or additional selection markers or express other or additional proteins. In particular, they can be such derivatives that express additional economically interesting proteins in addition to the inventively manufactured proteins.

It is well known to the person skilled in the art that the heterologous expression of genes is particularly successful when the chosen host cell of the organism, from which the original gene originates, is particularly closely related. Therefore, expression systems within the actinomycetes are also preferred systems for expression, streptomycetes being particularly preferred, among which Streptomyces coelicolor, Streptomyces avermitilis, Streptomyces lividans and in principle particularly those streptomycetes strains that by suitable selection or genetic manipulation are particularly suited for an industrial production of enzymes.

Preferred microorganisms are also those, which have been obtained by transformation with one of the vectors described above. This can concern cloning vectors, for example, which have been inserted into any bacterial strain for storage and/or modification. In general, such steps are generally widespread in the storage and further development of the genetic elements under consideration. As the relevant genetic elements from these microorganisms can be directly transferred into gram-negative bacteria for expression, the preceding transformation products can also fulfil the subject matter of the invention under consideration.

Eukaryotic cells can also be suitable for the production of interesting proteins. Examples of these are fungi like actinomycetes or yeasts like saccharomyces or kluyveromyces. For example, this can be particularly advantageous if the proteins should be subjected to specific modifications in connection with their synthesis, which permit such systems. For example, these include the binding of low molecular weight compounds such as docking membranes or oligosaccharides.

The host cells are cultivated and fermented in a conventional manner, for example in discontinuous or continuous systems. In the first case, a suitable nutrient medium is inoculated with the microorganisms and the product is harvested from the medium after an experimentally determined time. Continuous fermentations are characterized by the attainment of a flow equilibrium, in which, for a comparatively long time, cells partially die off but also grow again, and product can be removed from the medium.

Fermentation processes per se are well known from the prior art and represent the actual industrial production step; followed by a suitable purification method. All fermentation processes that are based on one of the above listed processes for manufacturing recombinant proteins, correspondingly represent preferred embodiments of this subject matter of the invention.

Here the optimal conditions for the production process, the host cells and/or the protein being produced have to be experimentally determined by the person skilled in the art with the help of the previously optimized culture conditions of the strains in question, for example in regard to fermentation volumes, medium composition, oxygen demand or stirring rate.

Fermentation processes, wherein the fermentation is carried out with a supply strategy, can also be considered. For this the ingredients of the medium that are used up by the ongoing cultivation are fed in; this is also known as a feed strategy. Considerable increases in both the cell density and in the dry biomass and/or above all in the activity of the protein of interest can be achieved by this.

In analogy with this, the fermentation can also be designed in such a way that unwanted metabolic products can be filtered off or be neutralized by the addition of buffer or matching counter ions.

The manufactured protein can be subsequently harvested from the fermentation medium. This fermentation process is preferred over the product purification from the dry mass, but requires the availability of suitable secretion markers and transport systems.

Without secretion, the purification of the proteins from the cell mass is possibly required and various processes are known for this, such as precipitation with e.g. ammonium sulfate or ethanol, or chromatographic purification, when required to homogeneity. However, the majority of the described techniques should be done with an enriched, stabilized preparation.

All of the above listed elements can be combined in processes to manufacture the interesting proteins. A great many possible combinations of process steps are conceivable for each interesting protein. The optimum process has to be determined experimentally for each particular case.

The amadoriases of interest can be produced by expression or cloning in sufficient quantities for industrial use.

The inventively employable amadoriases exhibit a pH optimum preferably in the weakly acid to alkaline range of about pH 4 to pH 12, particularly pH 6 to pH 11, preferably pH 7 to pH 10 and particularly preferably of about pH 7-8 and of about pH 9-10.

The activity of such enzymes is usually expressed in U, the unit corresponding to the quantity of enzyme that generates 1 μmol of hydrogen peroxide (H₂O₂) in 1 minute at a defined pH and a defined temperature.

The temperature optimum of the inventively employable amadoriases is in the range of about 20 to 60° C., particularly in the range 30 to 60° C. and particularly preferably in the range from about 30 to 40° C. for liquid products and from about 40 to 60° C. for powder products.

A further subject matter of the invention is the use of an amadoriase for the enzymatic decomposition of Amadori products. A preferred embodiment of the process according to the invention is the use in laundry detergent, rinse aid and cleaning compositions.

A further subject matter of the invention is the use of an amadoriase for the enzymatic generation of hydrogen peroxide in situ. A preferred embodiment of the process according to the invention is the use for bleaching, for color transfer inhibition and for disinfection.

The inventively employable amadoriases can be advantageously incorporated in body care products, hair care products, hair shampoos, compositions for oral care, dental care and dental prosthesis care, dental brace care products, cosmetics, therapeutics, laundry detergents, cleaning compositions, brightening agents, bleaching agents, disinfectants, rinse aids, detergents for hand washing, dish washing detergents, automatic dishwasher detergents and compositions for bleaching or disinfecting filter media, textiles, pelts, paper, hides or leather.

The laundry detergent, rinse aid, cleaning composition or bleaching agent according to the invention preferably possesses an amadoriase activity of 0.1 to 18 U/ml wash liquor, cleaning solution or user solution in the process; it is preferably present as a free flowing powder with a bulk density of 300 g/l to 1200 g/l, especially 500 g/l to 900 g/l. Alternatively however, it can also be in the form of a pasty or liquid laundry detergent, especially in the form of a non-aqueous liquid laundry detergent or a non-aqueous paste or in the form of an aqueous liquid laundry detergent or a water-containing paste.

The laundry detergent, rinse aid, cleaning composition or bleaching agent according to the invention can be packaged in air-impervious container, from which it is discharged shortly before use or during the wash cycle, in particular, the amadoriase can be encapsulated with a substance that is impermeable to the enzyme and/or its substrate at room temperature or in the absence of water, and which becomes permeable to the enzyme and/or its substrate under the conditions of use of the composition.

The laundry detergent, rinse aid or cleaning composition according to the invention comprises, in addition to the amadoriase,
5 wt. % to 70 wt. %, particularly 10 wt. % to 50 wt. % surfactant,
10 wt. % to 65 wt. %, particularly 12 wt. % to 60 wt. % of water-soluble, water-dispersible inorganic builder,
1 wt. % to 10 wt. %, particularly 2 wt. % to 8 wt. % water-soluble organic builders,
not more than 15 wt. % solid inorganic and/or organic acids or their acid salts,
not more than 5 wt. % sequestrants for heavy metals,
not more than 5 wt. % graying inhibitors,
not more than 5 wt. % color transfer inhibitors and
not more than 5 wt. % foam inhibitor.

Due to their high industrial importance, the different aspects and other ingredients of the inventive, i.e. the laundry detergent, rinse aid and cleaning compositions characterized by the above described amadoriases, will now be described in order to amplify in detail the above described particularly preferred embodiments.

There will be no overall distinction made between textiles and hard surfaces as the material to be washed. The available choices, in particular for the conditions required for the various ingredients, such as, for example temperature, pH, ion strength, redox conditions or mechanical influences, should be optimised for each cleaning problem. Thus, usual temperatures for laundry detergents and cleaning compositions are in the range 10° C. for manual compositions over 40° C. and 60° C. up to 95° C. for machine compositions or for industrial applications. As the temperature is mostly steplessly adjustable in modern washing machines and dishwashers, all intermediate steps of temperature are included. Preferably, the ingredients of the composition are harmonized with each other. Synergies in regard to the cleaning power are preferred.

An inventively employable amadoriase can be used both in compositions for large-scale end users or industrial users as well as in products for the private consumer, wherein all types of cleaning compositions established in the prior art also represent embodiments of the present invention. This includes for example concentrates and compositions to be used without dilution—for use on a commercial scale in washing machines or in hand washing or hand cleaning. These include, for example, laundry detergents for fabrics, carpets or natural fibers, for which the term “laundry detergent” is used in the present invention. These also include, for example, dishwashing detergents for dishwashing machines or manual dishwashing detergents or cleaners for hard surfaces, such as metal, glass, china, ceramic, tiles, stone, painted surfaces, plastics, wood or leather, for which the term “cleaning composition” is used in the present invention.

Embodiments of the present invention include all established and/or all appropriate presentation forms. These include for example solid, powdered, liquid, gel or pasty agents, optionally from a plurality of phases, compressed or non-compressed; further included are for example: extrudates, granulates, tablets or pouches, both in bulk and also packed in portions.

In inventive compositions, the inventively employable amadoriase is combined with individual or a plurality of the following ingredients: non-ionic, anionic and/or cationic surfactants, (optionally additional) bleaching agents, bleach activators, bleach catalysts, builders and/or cobuilders, solvents, thickeners, sequestrants, electrolytes, optical brighteners, graying inhibitors, corrosion inhibitors, especially silver protectants, soil release agents, color transfer inhibitors, foam inhibitors, abrasives, colorants, fragrances, antimicrobials, UV stabilizers, enzymes such as for example proteases, amylases, lipases, cellulases, hemicellulases or oxidases, stabilizers, especially enzyme stabilizers, and other components, which are known from the prior art.

Preferred non-ionic surfactants are alkoxylated, advantageously ethoxylated, particularly primary alcohols preferably containing 8 to 18 carbon atoms and, on average, 1 to 12 moles of ethylene oxide (EO) per mole of alcohol, in which the alcohol group may be linear or, preferably, methyl-branched in the 2-position or may contain linear and methyl-branched groups in the form of the mixtures typically present in oxoalcohol groups. In particular, however, alcohol ethoxylates with linear alcohol groups of natural origin with 12 to 18 carbon atoms, e.g. from coco-, palm-, tallow- or oleyl alcohol, and an average of 2 to 8 EO per mole alcohol are preferred. Exemplary preferred ethoxylated alcohols include C₁₂₋₁₄ alcohols with 3 EO or 4EO, C₉₋₁₁ alcohol with 7 EO, C₁₃₋₁₅ alcohols with 3 EO, 5 EO, 7 EO or 8 EO, C₁₂₋₁₈ alcohols with 3 EO, 5 EO or 7 EO and mixtures thereof, as well as mixtures of C₁₂₋₁₄ alcohol with 3 EO and C₁₂₋₁₈ alcohol with 5 EO. The cited degrees of ethoxylation constitute statistically average values that can be a whole or a fractional number for a specific product. Preferred alcohol ethoxylates have a narrowed homolog distribution (narrow range ethoxylates, NRE). In addition to these non-ionic surfactants, fatty alcohols with more than 12 EO can also be used. Examples of these are tallow fatty alcohol with 14 EO, 25 EO, 30 EO or 40 EO.

Another class of preferred non-ionic surfactants which may be used, either as the sole non-ionic surfactant or in combination with other non-ionic surfactants are alkoxylated, preferably ethoxylated or ethoxylated and propoxylated fatty acid alkyl esters preferably containing 1 to 4 carbon atoms in the alkyl chain, in particular fatty acid methyl esters.

A further class of non-ionic surfactants, which can be advantageously used, are the alkyl polyglycosides (APG). Suitable alkyl polyglycosides satisfy the general Formula RO(G)_{z} where R is a linear or branched, particularly 2-methyl-branched, saturated or unsaturated aliphatic group containing 8 to 22 and preferably 12 to 18 carbon atoms and G stands for a glycose unit containing 5 or 6 carbon atoms, preferably glucose. Here, the degree of glycosidation z is between 1.0 and 4.0, preferably between 1.0 and 2.0 and particularly between 1.1 and 1.4. Linear alkyl polyglucosides are preferably employed, that is alkyl polyglycosides, in which the polyglycosyl group is a glucose group and the alkyl group is an n-alkyl group.

Non-ionic surfactants of the amine oxide type, for example N-cocoalkyl-N,N-dimethylamine oxide and N-tallow alkyl-N,N-dihydroxyethylamine oxide, and the fatty acid alkanolamides may also be suitable. The quantity of these non-ionic surfactants is preferably no more than the quantity in which the ethoxylated fatty alcohols are used and, particularly no more than half that quantity.

Exemplary suitable anionic surfactants are those of the sulfonate and sulfate type. Suitable surfactants of the sulfonate type are, advantageously C₉₋₁₃ alkylbenzene sulfonates, olefin sulfonates, i.e. mixtures of alkene- and hydroxyalkane sulfonates, and disulfonates, as are obtained, for example, from C₁₂₋₁₈ monoolefins having a terminal or internal double bond, by sulfonation with gaseous sulfur trioxide and subsequent alkaline or acidic hydrolysis of the sulfonation products. Those alkane sulfonates, obtained from C₁₂₋₁₈ alkanes by sulfochlorination or sulfoxidation, for example, with subsequent hydrolysis or neutralization, are also suitable. The esters of a-sulfofatty acids (ester sulfonates), e.g. the α-sulfonated methyl esters of hydrogenated coco-, palm nut- or tallow acids are likewise suitable.

Further suitable anionic surfactants are sulfated fatty acid esters of glycerine. They include the mono-, di- and triesters and also mixtures of them, such as those obtained by the esterification of a monoglycerine with 1 to 3 moles fatty acid or the transesterification of triglycerides with 0.3 to 2 moles glycerine. Preferred sulfated fatty acid esters of glycerine in this case are the sulfated products of saturated fatty acids with 6 to 22 carbon atoms, for example caproic acid, caprylic acid, capric acid, myristic acid, lauric acid, palmitic acid, stearic acid or behenic acid.

Preferred alk(en)yl sulfates are the alkali metal and especially sodium salts of the sulfuric acid half-esters derived from the C₁₂-C₁₈ fatty alcohols, for example from coconut butter alcohol, tallow alcohol, lauryl, myristyl, cetyl or stearyl alcohol or from C₁₀-C₂₀ oxo alcohols and those half-esters of secondary alcohols of these chain lengths. Additionally preferred are alk(en)yl sulfates of the said chain lengths, which contain a synthetic, straight-chained alkyl group produced on a petrochemical basis and which show similar degradation behaviour to the suitable compounds based on fat chemical raw materials. The C₁₂-C₁₅ alkyl sulfates and C₁₂-C₁₅ alkyl sulfates and C₁₄-C₁₅ alkyl sulfates are preferred on the grounds of laundry performance. 2,3-Alkyl sulfates are also suitable anionic surfactants.

Sulfuric acid mono-esters derived from straight-chained or branched C₇₋₂₁ alcohols ethoxylated with 1 to 6 moles ethylene oxide are also suitable, for example 2-methyl-branched C₉₋₁₁ alcohols with an average of 3.5 mole ethylene oxide (EO) or C₁₂₋₁₈ fatty alcohols with 1 to 4 EO. Due to their high foaming performance, they are only used in fairly small quantities in cleaning compositions, for example in amounts of up to 5% by weight, usually from 1 to 5% by weight.

Other suitable anionic surfactants are the salts of alkylsulfosuccinic acids, which are also referred to as sulfosuccinates or esters of sulfosuccinic acid and the monoesters and/or di-esters of sulfosuccinic acid with alcohols, preferably fatty alcohols and especially ethoxylated fatty alcohols. Preferred sulfosuccinates comprise C₈₋₁₈ fatty alcohol groups or mixtures of them. Especially preferred sulfosuccinates comprise a fatty alcohol group derived from ethoxylated fatty alcohols and may be considered as non-ionic surfactants (see description below). Once again the particularly preferred sulfosuccinates are those, whose fatty alcohol groups are derived from ethoxylated fatty alcohols with narrow range homolog distribution. It is also possible to use alk(en)ylsuccinic acids with preferably 8 to 18 carbon atoms in the alk(en)yl chain, or salts thereof.

Soaps in particular can be considered as further anionic surfactants. Saturated fatty acid soaps are suitable, such as the salts of lauric acid, myristic acid, palmitic acid, stearic acid, hydrogenated erucic acid and behenic acid, and especially soap mixtures derived from natural fatty acids such as coconut oil fatty acid, palm kernel oil fatty acid or tallow fatty acid.

Anionic surfactants, including soaps may be in the form of their sodium, potassium or ammonium salts or as soluble salts of organic bases, such as mono-, di- or triethanolamine. Preferably, the anionic surfactants are in the form of their sodium or potassium salts, especially in the form of the sodium salts.

The surfactants can be comprised in the inventive cleaning compositions or laundry detergents in a total amount of preferably 5 to 50 wt. %, particularly 8 to 30 wt. %, based on the finished composition.

Inventive compositions can comprise additional bleaching agent. Among the compounds, which serve as bleaches and liberate H₂O₂ in water, sodium percarbonate, sodium perborate tetrahydrate and sodium perborate monohydrate are of particular importance. Examples of further bleaching agents that may be used are peroxypyrophosphates, citrate perhydrates and H₂O₂-liberating peracidic salts or peracids, such as persulfates or persulfuric acid. The urea peroxyhydrate percarbamide that can be described by the formula H₂N—CO—NH₂H₂O₂ is also suitable. Particularly when agents are used to clean hard surfaces, for example in automatic dishwashers, they can, if desired, also comprise bleaching agents from the group of the organic bleaching agents, although in principal they can also be used for washing textiles. Typical organic bleaching agents are the diacyl peroxides, such as e.g. dibenzoyl peroxide. Further typical organic bleaching agents are the peroxy acids, wherein the alkylperoxy acids and the arylperoxy acids may be named as examples. Preferred representatives that can be added are peroxybenzoic acid and ring-substituted derivatives thereof, such as alkyl peroxybenzoic acids, but also peroxy-α(-naphthoic acid and magnesium monoperphthalate, the aliphatic or substituted aliphatic peroxy acids, such as peroxylauric acid, peroxystearic acid, ε-phthalimidoperoxycaproic acid (phthalimidoperoxyhexanoic acid PAP), o-carboxybenzamidoperoxycaproic acid, N-nonenylamidoperadipic acid and N-nonenylamidopersuccinates and aliphatic and araliphatic peroxydicarboxylic acids, such as 1,12-diperoxycarboxylic acid, 1,9-diperoxyazelaic acid, diperoxysebacic acid, diperoxybrassylic acid, diperoxyphthalic acids, 2-decyldiperoxybutane-1,4-dioic acid, N,N-terephthaloyl-di(6-aminopercaproic acid).

The bleaching agent content of the composition is preferably 1 to 40 wt. % and particularly 10 to 20 wt. %, perborate monohydrate or percarbonate being advantageously used.

The preparations can also comprise bleach activators in order to achieve an improved bleaching action for washing temperatures of 60° C. and below and particularly during the pre-treatment wash. Bleach activators, which can be used, are compounds which, under perhydrolysis conditions, yield aliphatic peroxycarboxylic acids having preferably 1 to 10 carbon atoms, in particular 2 to 4 carbon atoms, and/or optionally substituted perbenzoic acid. Substances, which carry O-acyl and/or N-acyl groups of said number of carbon atoms and/or optionally substituted benzoyl groups, are suitable. Preference is given to polyacylated alkylenediamines, in particular tetraacetyl ethylenediamine (TAED), acylated triazine derivatives, in particular 1,5-diacetyl-2,4-dioxohexahydro-1,3,5-triazine (DADHT), acylated glycolurils, in particular 1,3,4,6-tetraacetyl glycoluril (TAGU), N-acylimides, in particular N-nonanoyl succinimide (NOSI), acylated phenol sulfonates, in particular n-nonanoyl- or isononanoyloxybenzene sulfonate (n- or iso-NOBS), carboxylic acid anhydrides, in particular phthalic anhydride, acylated polyhydric alcohols, in particular triacetin, ethylene glycol diacetate and 2,5-diacetoxy-2,5-dihydrofuran and the enol esters known from the German Patent applications DE 196 16 693 and DE 196 16 767 and acetylated sorbitol and mannitol or their mixtures (SORMAN) described in the European Patent application EP 0 525 239, acylated sugar derivatives, in particular pentaacetyl glucose (PAG), pentaacetyl fructose, tetraacetyl xylose and octaacetyl lactose as well as acetylated, optionally N-alkylated glucamine and gluconolactone, triazole or triazole derivatives and/or particulate caprolactams and/or caprolactam derivatives, preferably N-acylated lactams, for example N-benzoyl caprolactam and N-acetyl caprolactam. Hydrophilically substituted acyl acetals and acyl lactams are also preferably used. Combinations of conventional bleach activators may also be used. Nitrile derivatives such as cyanopyridines, nitrilequats, for example N-alkylammonium acetonitrile, and/or cyanamide derivatives can also be used. Preferred bleach activators are sodium 4-(octanoyloxy)benzene sulfonate, n-nonanoyl- or isononanoyloxybenzene sulfonate (n- or iso-NOBS), undecenoyloxybenzene sulfonate (UDOBS), sodium dodecanoyloxybenzene sulfonate (DOBS), decanoyloxybenzoic acid (DOBA, OBC 10) and/or dodecanoyloxybenzene sulfonate (OBS 12), and N-methylmorpholinum acetonitrile (MMA).

These types of bleach activators are comprised in the usual quantity range of 0.01 to 20 wt. %, preferably in amounts of 0.1 wt. % to 15 wt. %, particularly 1 wt. % to 10 wt. %, based on the total composition.

In the context of the present application, further preferred added bleach activators are compounds from the group of the cationic nitriles, particularly cationic nitriles of the Formula

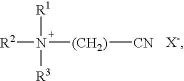

in which R¹ stands for —H, —CH₃, a C₂₋₂₄-alkyl or alkenyl group, a substituted C₂₋₂₄ alkyl or alkenyl group having at least one substituent from the group of —Cl, —Br, —OH, —NH₂, —CN, an alkylaryl or alkenylaryl group having a C₁₋₂₄ alkyl group or for a substituted alkylaryl or alkenylaryl group having a C₁₋₂₄ alkyl group and at least one further substituent on the aromatic ring, R² and R³ independently of one another are selected from —CH₂—CN, —CH₃, —CH₂—CH₃, —CH₂—CH₂—CH₃, —CH(CH₃)—CH₃, —CH₂—OH, —CH₂—CH₂—OH, —CH(OH)—CH₃, —CH₂—CH₂—CH₂—OH, —CH₂—CH(OH)—CH₃, —CH(OH)—CH₂—CH₃, —(CH₂CH₂—O)ₙH with n=1, 2, 3, 4, 5 or 6 and X is an anion.

A cationic nitrile of the following Formula is particularly preferred

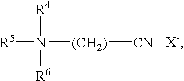

in which R⁴, R⁵ and R⁶ independently of one another are selected from —CH₃, —CH₂—CH₃, —CH₂—CH₂—CH₃, —CH(CH₃)—CH₃, wherein R⁴ can also be —H and X is an anion, wherein preferably R⁵═R⁶=—CH₃ and in particular R⁴=R⁵=R⁶=—CH₃ and compounds of the formulae (CH₃)₃N⁽⁺⁾CH₂—CNX⁻, (CH₃CH₂)₃N⁽⁺⁾CH₂—CNX⁻, (CH₃CH₂CH₂)₃N⁽⁺⁾CH₂—CNX⁻, (CH₃CH(CH₃))₃N⁽⁺⁾CH₂—CNX⁻, or (HO—CH₂—CH₂)₃N⁽⁺⁾CH₂—CNX⁻ are particularly preferred, wherein once again from the group of these substances the cationic nitrile of the formula (CH₃)₃N⁽⁺⁾CH₃X⁻, in which X⁻ stands for an anion selected from the group chloride, bromide, iodide, hydrogen sulfate, methosulfate, p-toluene sulfonate (tosylate) or xylene sulfonate is particularly preferred.

In addition to, or instead of the conventional bleach activators mentioned above, so-called bleach catalysts may also be incorporated. These substances are bleach-boosting transition metal salts or transition metal complexes such as, for example, manganese-, iron-, cobalt-, ruthenium- or molybdenum-salen complexes or -carbonyl complexes. Manganese, iron, cobalt, ruthenium, molybdenum, titanium, vanadium and copper complexes with nitrogen-containing tripod ligands, as well as cobalt-, iron-, copper- and ruthenium-amine complexes may also be employed as the bleach catalysts.

Complexes of manganese in the valence state II, III, IV or V are particularly preferably employed, which preferably comprise one or a plurality of macrocyclic ligands with the donor functions N, NR, PR, O and/or S. Ligands having nitrogen donor functions are preferably employed. In this regard, it is particularly preferred to incorporate bleach catalysts into the compositions according to the invention, which comprise 1,4,7-trimethyl-1,4,7-triazacyclononane (Me-TACN), 1,4,7-triazacyclononane (TACN), 1,5,9-trimethyl-1,5,9-triazacyclododecane (Me-TACD), 2-methyl-1,4,7-trimethyl-1,4,7-triazacyclononane (Me/Me-TACN) and/or 2-methyl-1,4,7-triazacyclononane (Me/TACN) as the macromolecular ligands. Suitable manganese complexes are for example [Mn^{III}2(μ-O)₁(μ-OAc)₂(TACN)₂](CIO₄)₂, [Mn^{III}Mn^{IV}(μ-O)₂(μ-OAc)₁(TACN)₂](BPh₄)₂, [Mn^{IV}₄(μ-O)₆(TACN)₄](CIO₄)₄, [Mn^{III}₂(μ-O)₁(μ-OAc₂(Me-TACN)₂](CIO₄)₂, [Mn^{III}Mn^{IV}(μ-O)₁(μ-OAc)₂(Me-TACN)₂](CIO₄)S, [Mn^{IV}₂(μ-O)₃(Me-TACN)₂](PF₆)₂ and [Mn^{IV}₂(μ-O)₂(Me/Me-TACN)₂](PF₆)₂ (OAc=OC(O)CH₃).

Bleach catalysts can be added in usual amounts, preferably in an amount of up to 5 wt. %, particularly from 0.0025 wt. % to 1 wt. % and particularly preferably from 0.01 to 0.25 wt. %, each based on the total weight of the bleach activator-containing composition. However, in special cases more bleach activator may also be employed.

According to WO 99/63038, acetonitrile derivatives, and according to WO 99/63041, bleach activating transition metal complexes in combination with amylases, are also capable of developing a bleach activating effect.

Enzymes can also be employed as the bleach activator, which with hydrogen peroxide catalyze a perhydrolysis reaction of a substrate to yield peracids. Such enzymes can derive from lipases or cutinases, or also from esterases or proteases, in the case where namely a perhydrolysis reaction completely or partially occurs instead of hydrolysis. Such perhydrolases are described for example in WO2005/056782 (based on an esterase) or WO2004/058961 (based on a modified subtilisin Carlsberg). The use of the inventive bleaching system with the aid of perhydrolases is also a component of the present invention. Accordingly, the compositions according to the invention can comprise lipases or cutinases, particularly due to their triglyceride cleaving activities, but also in order to produce in situ peracids from suitable preliminary steps. These include for example the available or further developed lipases originating from Humicola lanuginosa (Thermomyces lanuginosus), particularly those with the amino acid substitution D96L. They are commercialized, for example by the Novozymes Company under the trade names Lipolase®, Lipolase® Ultra, LipoPrime®, Lipozyme® and Lipex®. Moreover, suitable cutinases, for example are those that were originally isolated from Fusarium solani pisi and Humicola insolens. Likewise useable lipases are available from the Amano Company under the designations Lipase CE®, Lipase P®, Lipase B®, and Lipase CES®, Lipase AKG®, Bacillis sp. Lipase®, Lipase AP®, Lipase M-AP® and Lipase AML®. Suitable lipases or cutinases whose starting enzymes were originally isolated from Pseudomonas mendocina and Fusarium solanii are for example available from the Genencor Company. Further important commercial products that may be mentioned are the commercial preparations M1 Lipase® and Lipomax® originally from the Gist-Brocades Company, and the commercial enzymes from the Meito Sangyo KK Company, Japan under the names Lipase MY-30®, Lipase OF® and Lipase PL® as well as the product Lumafast® from the Genencor Company.

Generally, inventive compositions comprise one or more builders, in particular zeolites, silicates, carbonates, organic cobuilders and—where there are no ecological grounds against their use—also phosphates. The last are particularly preferred builders employed in cleaning compositions for automatic dishwashers.

Suitable silicate builders are the crystalline, layered sodium silicates corresponding to the general formula NaMSiₓO₂ₓ₊₁yH₂O, wherein M is sodium or hydrogen, x is a number from 1.6 to 4. preferably 1.9 to 4.0 and y is a number from 0 to 20, preferred values for x being 2, 3 or 4. These types of crystalline layered silicates are described, for example, in the European Patent application EP 0 164 514. Preferred crystalline layered silicates of the given formula are those in which M stands for sodium and x assumes the values 2 or 3. In particular, both β- and δ-sodium disilicates Na₂Si₂O₅.yH₂O are preferred. These types of compounds are commercially available, for example, under the designation SKS® (Clariant). SKS-6® is mainly a 5-sodium disilicate with the formula Na₂Si₂O₅.yH₂O, and SKS-7® is mainly the β-sodium disilicate. On reaction with acids (e.g. citric acid or carbonic acid), δ-sodium silicate affords Kanemit NaHSi₂O₅, commercially available under the trade names SKS-9® and SKS-10® (Clariant). It can also be advantageous to chemically modify these layered silicates. The alkalinity, for example, of the layered silicates can be suitably modified. In comparison with the δ-sodium disilicate, layered silicates, doped with phosphate or carbonate, exhibit a different crystal morphology, dissolve more rapidly and show an increased calcium binding ability. Thus, layered silicates of the general formula xNa₂OySiO₂zP₂O₅ in which the ratio x to y corresponds to a number 0.35 to 0.6, the ratio x to z a number from 1.75 to 1200 and the ratio y to z a number from 4 to 2800, are described in the patent application DE 196 01 063. The solubility of the layered silicates can also be increased by employing particularly finely dispersed layered silicates. Compounds of the crystalline layered silicates with other ingredients can also be used. Compounds with cellulose derivatives, which possess advantages in the disintegration action, and which are particularly used in detergent tablets, as well as compounds with polycarboxylates, for example citric acid or polymeric polycarboxylates, for example copolymers of acrylic acid can be particularly cited in this context.

Other useful builders are amorphous sodium silicates with a modulus (Na₂O:SiO₂ ratio) of 1:2 to 1:3.3, preferably 1:2 to 1:2.8 and more preferably 1:2 to 1:2.6, which dissolve with a delay and exhibit multiple wash cycle properties. The delay in dissolution compared with conventional amorphous sodium silicates can have been obtained in various ways, for example by surface treatment, compounding, compressing/compacting or by over-drying. In the context of this invention, the term “amorphous” also means “X-ray amorphous”. In other words, the silicates do not produce any of the sharp X-ray reflexes typical of crystalline substances in X-ray diffraction experiments, but at best one or more maxima of the scattered X-radiation, which have a width of several degrees of the diffraction angle. However, particularly good builder properties may even be achieved where the silicate particles produce indistinct or even sharp diffraction maxima in electron diffraction experiments. This is to be interpreted to mean that the products have microcrystalline regions between 10 and a few hundred nm in size, values of up to at most 50 nm and especially up to at most 20 nm being preferred. Compacted/densified amorphous silicates, compounded amorphous silicates and over dried X-ray-amorphous silicates are particularly preferred.

An optionally suitable fine crystalline, synthetic zeolite containing bound water, is preferably zeolite A and/or P. Zeolite MAP® (commercial product of the Crosfield company), is particularly preferred as the zeolite P. However, zeolite X and mixtures of A, X and/or P are also suitable. Commercially available and preferably used in the context of the present invention is, for example, also a co-crystallizate of zeolite X and zeolite A (ca. 80 wt. % zeolite X), which is marketed by CONDEA Augusta S.p.A. under the trade name VEGOBOND AX® and which can be described by the Formula

nNa₂O.(1-n)K₂O.Al₂O₃.(2-2.5)SiO₂.(3.5-5.5)H₂O

Suitable zeolites have a mean particle size of less than 10 μm (volume distribution, as measured by the Coulter Counter Method) and contain preferably 18 to 22% by weight and more preferably 20 to 22% by weight of bound water.

Naturally, the generally known phosphates can also be added as builders, in so far that their use should not be avoided on ecological grounds. In the detergent and cleaning agent industry, among the many commercially available phosphates, the alkali metal phosphates are the most important and pentasodium or pentapotassium triphosphates (sodium or potassium tripolyphosphate) are particularly preferred.

“Alkali metal phosphates” is the collective term for the alkali metal (more particularly sodium and potassium) salts of the various phosphoric acids, in which metaphosphoric acids (HPO₃)ₙ and orthophosphoric acid (H₃PO₄) and representatives of higher molecular weight can be differentiated. The phosphates combine several inherent advantages: they act as alkalinity sources, prevent lime deposits on machine parts and lime incrustations in fabrics and, in addition, contribute towards the cleaning effect.

Sodium dihydrogen phosphate NaH₂PO₄ exists as the dihydrate (density 1.91 gcm⁻³, melting point 60° C.) and as the monohydrate (density 2.04 gcm⁻³). Both salts are white, readily water-soluble powders that on heating, lose the water of crystallization and at 200° C. are converted into the weakly acidic diphosphate (disodium hydrogen diphosphate, Na₂H₂P₂O₇) and, at higher temperatures into sodium trimetaphosphate (Na₃P₃O₉) and Maddrell's salt (see below). NaH₂PO₄ shows an acidic reaction. It is formed by adjusting phosphoric acid with sodium hydroxide to a pH value of 4.5 and spraying the resulting “mash”. Potassium dihydrogen phosphate (primary or monobasic potassium phosphate, potassium biphosphate, KDP), KH₂PO₄, is a white salt with a density of 2.33 gcm⁻³, has a melting point of 253° C. [decomposition with formation of potassium polyphosphate (KPO₃)ₓ] and is readily soluble in water.

Disodium hydrogen phosphate (secondary sodium phosphate), Na₂HPO₄, is a colorless, very readily water-soluble crystalline salt. It exists in anhydrous form and with 2 mol (density 2.066 gcm⁻³, water loss at 95° C.), 7 mol (density 1.68 gcm⁻³, melting point 48° C. with loss of 5H₂O) and 12 mol of water (density 1.52 gcm⁻³, melting point 35° C. with loss of 5H₂O), becomes anhydrous at 100° C. and, on fairly intensive heating, is converted into the diphosphate Na₄P₂O₇. Disodium hydrogen phosphate is prepared by neutralization of phosphoric acid with soda solution using phenolphthalein as the indicator. Dipotassium hydrogen phosphate (secondary or dibasic potassium phosphate), K₂HPO₄, is an amorphous white salt, which is readily soluble in water.

Trisodium phosphate, tertiary sodium phosphate, Na₃PO₄, consists of colorless crystals with a density of 1 62 gcm⁻³ and a melting point of 73-76° C. (decomposition) as the dodecahydrate, a melting point of 100° C. as the decahydrate (corresponding to −19-20% P₂O₅) and in anhydrous form (corresponding to 39-40% P₂O₅) a density of 2.536 gcm⁻³. Trisodium phosphate is readily soluble in water with an alkaline reaction and is manufactured by evaporating a solution of exactly 1 mole disodium phosphate and 1 mole NaOH. Tripotassium phosphate (tertiary or tribasic potassium phosphate), K₃PO₄, is a white deliquescent granular powder with a density of 2.56 gcm⁻³, has a melting point of 1340° C. and is readily soluble in water through an alkaline reaction. It is produced by e.g. heating Thomas slag with carbon and potassium sulfate. Despite their higher price, the more readily soluble and therefore highly effective potassium phosphates are often preferred to corresponding sodium compounds in the detergent industry.

Tetrasodium diphosphate (sodium pyrophosphate), Na₄P₂O₇, exists in anhydrous form (density 2.534 gcm⁻³, melting point 988° C., a figure of 880° C. has also been mentioned) and as the decahydrate (density 1.815-1.836 gcm⁻³, melting point 94° C. with loss of water). Both substances are colorless crystals that dissolve in water with an alkaline reaction. Na₄P₂O₇ is formed when disodium phosphate is heated to more than 200° C. or by reacting phosphoric acid with soda in a stoichiometric ratio and spray drying the solution. The decahydrate complexes heavy metal salts and hardness salts and, hence, reduces the hardness of water. Potassium diphosphate (potassium pyrophosphate), K₄P₂O₇, exists in the form of the trihydrate and is a colorless hygroscopic powder with a density of 2.33 gcm⁻³, which is soluble in water, the pH of a 1% solution at 25° C. being 10.4.

Relatively high molecular weight sodium and potassium phosphates are formed by condensation of NaH₂PO₄ or KH₂PO₄. They may be divided into cyclic types, namely the sodium and potassium metaphosphates, and chain types, the sodium and potassium polyphosphates. The chain types in particular are known by various different names: fused or calcined phosphates, Graham's salt, Kurrol's salt and Maddrell's salt. All higher sodium and potassium phosphates are known collectively as condensed phosphates.

The industrially important pentasodium triphosphate, Na₅P₃O₁₀ (sodium tripolyphosphate), is anhydrous or crystallizes with 6H₂O to a non-hygroscopic white water-soluble salt which and which has the general formula NaO—[P(O)(ONa)—O]ₙ—Na where n=3. Around 17 g of the salt free from water of crystallization dissolve in 100 g of water at room temperature, around 20 g at 60° C. and around 32 g at 100° C. After heating the solution for 2 hours to 100° C., around 8% orthophosphate and 15% diphosphate are formed by hydrolysis. In the preparation of pentasodium triphosphate, phosphoric acid is reacted with soda solution or sodium hydroxide in a stoichiometric ratio and the solution is spray-dried. Similarly to Graham's salt and sodium diphosphate, pentasodium triphosphate solubilizes many insoluble metal compounds (including lime soaps, etc.). K₅P₃O₁₀ (potassium tripolyphosphate), is marketed for example in the form of a 50% by weight solution (>23% P₂O₅, 25% K₂O). The potassium polyphosphates are widely used in the laundry detergent and cleaning industry. Sodium potassium tripolyphosphates also exist and are also usable in the scope of the present invention. They are formed for example when sodium trimetaphosphate is hydrolyzed with KOH:

(NaPO₃)₃+2KOH->Na₃K₂P₃O₁₀+H₂O

According to the invention, they may be used in exactly the same way as sodium tripolyphosphate, potassium tripolyphosphate or mixtures thereof. Mixtures of sodium tripolyphosphate and sodium potassium tripolyphosphate or mixtures of potassium tripolyphosphate and sodium potassium tripolyphosphate or mixtures of sodium tripolyphosphate and potassium tripolyphosphate and sodium potassium tripolyphosphate may also be used in accordance with the invention.

Organic co builders, which may be used in the detergents and cleaning agents according to the invention, include, in particular, polycarboxylates or polycarboxylic acids, polymeric polycarboxylates, polyaspartic acid, polyacetals, optionally oxidized dextrins, other organic co builders (see below) and phosphonates. These classes of substances are described below.

Useful organic builders are, for example, the polycarboxylic acids usable in the form of their sodium salts, polycarboxylic acids in this context being understood to be carboxylic acids that carry more than one acid function. These include, for example, citric acid, adipic acid, succinic acid, glutaric acid, malic acid, tartaric acid, maleic acid, fumaric acid, sugar acids, aminocarboxylic acids, nitrilotriacetic acid (NTA), providing its use is not ecologically unsafe, and mixtures thereof. Preferred salts are the salts of polycarboxylic acids such as citric acid, adipic acid, succinic acid, glutaric acid, tartaric acid, sugar acids and mixtures thereof.

Acids per se can also be used. Besides their building effect, the acids also typically have the property of an acidifying component and, hence also serve to establish a relatively low and mild pH in washing or cleaning agents, when the pH, which results from the mixture of other components, is not wanted. Acids that are system-compatible and environmentally compatible such as citric acid, acetic acid, tartaric acid, malic acid, glycolic acid, succinic acid, glutaric acid, adipic acid, gluconic acid and mixtures thereof are particularly mentioned in this regard. However, mineral acids, particularly sulfuric acid or bases, particularly ammonium or alkali metal hydroxides can also serve as pH regulators. These types of regulators are preferably comprised in the inventive agents in amounts of not more than 20 wt. %, particularly from 1.2 wt. % to 17 wt. %.

Other suitable builders are polymeric polycarboxylates, i.e. for example the alkali metal salts of polyacrylic or polymethacrylic acid, for example those with a relative molecular weight of 500 to 70 000 g/mol.

The molecular weights mentioned in this specification for polymeric polycarboxylates are weight-average molecular weights M_{w} of the particular acid form which, fundamentally, were determined by gel permeation chromatography (GPC), equipped with a UV detector. The measurement was carried out against an external polyacrylic acid standard, which provides realistic molecular weight values by virtue of its structural similarity to the polymers investigated. These values differ significantly from the molecular weights measured against polystyrene sulfonic acids as the standard. The molecular weights measured against polystyrene sulfonic acids are generally significantly higher than the molecular weights mentioned in this specification.

Particularly suitable polymers are polyacrylates, which preferably have a molecular weight of 2000 to 20 000 g/mol. By virtue of their superior solubility, preferred representatives of this group are the short-chain polyacrylates, which have molecular weights of 2000 to 10 000 g/mol and, more particularly, 3000 to 5000 g/mol.

Further suitable copolymeric polycarboxylates are particularly those of acrylic acid with methacrylic acid and of acrylic acid or methacrylic add with maleic acid. Copolymers of acrylic acid with maleic acid, which comprise 50 to 90 wt. % acrylic acid and 50 to 10 wt. % maleic acid, have proven to be particularly suitable. Their relative molecular weight, based on free acids, generally ranges from 2 000 to 70 000 g/mol, preferably 20 000 to 50 000 g/mol and especially 30 000 to 40 000 g/mol. The (co)polymeric polycarboxylates can be used either as powders or as aqueous solutions. The (co)polymeric polycarboxylate content of the compositions is preferably from 0.5 to 20% by weight, in particular from 1 to 10% by weight.

In order to improve the water solubility, the polymers can also comprise allylsulfonic acids, such as for example, allyloxybenzene sulfonic acid and methallyl sulfonic acid as monomers.

Other particularly preferred polymers are biodegradable polymers of more than two different monomer units, for example those which contain salts of acrylic acid and maleic acid and vinyl alcohol or vinyl alcohol derivatives as monomers or those which contain salts of acrylic acid and 2-alkylallyl sulfonic acid and sugar derivatives as monomers.

Other preferred copolymers are those, which preferably contain acrolein and acrylic acid/acrylic acid salts or acrolein and vinyl acetate as monomers.

Similarly, other preferred builders are polymeric amino dicarboxylic acids, salts or precursors thereof. Polyaspartic acids or their salts and derivatives are particularly preferred.

Further preferred builders are polyacetals that can be obtained by treating dialdehydes with polyol carboxylic acids that possess 5 to 7 carbon atoms and at least 3 hydroxyl groups. Preferred polyacetals are obtained from dialdehydes like glyoxal, glutaraldehyde, terephthalaldehyde as well as their mixtures and from polycarboxylic acids like gluconic acid and/or glucoheptonic acid.

Further suitable organic builders are dextrins, for example oligomers or polymers of carbohydrates that can be obtained by the partial hydrolysis of starches. The hydrolysis can be carried out using typical processes, for example acidic or enzymatic catalyzed processes. The hydrolysis products preferably have average molecular weights in the range 400 to 500 000 g/mol. A polysaccharide with a dextrose equivalent (DE) of 0.5 to 40 and, more particularly, 2 to 30 is preferred, the DE being an accepted measure of the reducing effect of a polysaccharide by comparison with dextrose, which has a DE of 100. Both maltodextrins with a DE between 3 and 20 and dry glucose syrups with a DE between 20 and 37 and also so-called yellow dextrins and white dextrins with relatively high molecular weights of 2000 to 30 000 g/mol may be used.

The oxidized derivatives of such dextrins concern their reaction products with oxidizing agents that are capable of oxidizing at least one alcohol function of the saccharide ring to the carboxylic acid function. Particularly preferred organic builders for inventive compositions are oxidized starches and their derivatives from the applications EP 472 042, WO 97/25399, and EP 755 944.

Oxydisuccinates and other derivatives of disuccinates, preferably ethylenediamine disuccinate are also further suitable cobuilders. Ethylenediamine-N,N′-disuccinate (EDDS) is preferably used here in the form of its sodium or magnesium salts. In this context, glycerine disuccinates and glycerine trisuccinates are also preferred. Suitable addition quantities in zeolite-containing and/or silicate-containing formulations range from 3 to 15% by weight.

Other useful organic co-builders are, for example, acetylated hydroxycarboxylic acids and salts thereof which may optionally be present in lactone form and which contain at least 4 carbon atoms, at least one hydroxy group and at most two acid groups.

The phosphonates represent a further class of substances with cobuilder properties. In particular, they are hydroxyalkane phosphonates or aminoalkane phosphonates. Among the hydroxyalkane phosphonates, 1-hydroxyethane-1,1-diphosphonate (HEDP) is of particular importance as the cobuilder. It is normally added as the sodium salt, the disodium salt reacting neutral and the tetrasodium salt reacting alkaline (pH 9). Ethylenediamine tetramethylene phosphonate (EDTMP), diethylenetriamine pentamethylene phosphonate (DTPMP) and their higher homologs are preferably chosen as aminoalkane phosphonates. They are preferably added in the form of the neutral-reacting sodium salts, e.g. as the hexasodium salt of EDTMP or as the hepta and octasodium salt of DTPMP. Of the class of phosphonates, HEDP is preferably used as the builder. The aminoalkane phosphonates additionally possess a pronounced ability to complex heavy metals. Accordingly, it can be preferred, particularly where the agents also contain bleach, to use aminoalkane phosphonates, particularly DTPMP, or mixtures of the mentioned phosphonates.

In addition, any compounds capable of forming complexes with alkaline earth metal ions may be used as co-builders.

Builders can be comprised in the inventive compositions optionally in quantities of up to 90% by weight. They are preferably comprised in quantities of up to 75% by weight. Inventive laundry detergents possess builder contents of particularly 5 wt. % to 50 wt. %. In inventive compositions for cleaning hard surfaces, in particular for automatic dishwashing of tableware, the content of builders is particularly 5 wt. % to 88 wt. %, wherein in this type of composition, no water-insoluble builders are employed. In a preferred embodiment, the inventive composition, particularly for automatic dishwashers, comprises 20 wt. % to 40 wt. % of water-soluble organic builders, particularly alkali citrate, 5 wt. % to 15 wt. % alkali carbonate and 20 wt. % to 40 wt. % alkali disilicate.

Solvents that can be added to the liquid to gel-like compositions of laundry detergent and cleaning compositions originate, for example, from the group of mono- or polyhydric alcohols, alkanolamines or glycol ethers, in so far that they are miscible with water in the defined concentrations. Preferably, the solvents are selected from ethanol, n- or i-propanol, butanols, ethylene glycol methyl ether, ethylene glycol ethyl ether, ethylene glycol propyl ether, ethylene glycol mono-n-butyl ether, diethylene glycol methyl ether, diethylene glycol ethyl ether, propylene glycol methyl-, -ethyl- or -propyl ether, dipropylene glycol methyl-, or -ethyl ether, methoxy-, ethoxy- or butoxy triglycol, 1-butoxyethoxy-2-propanol, 3-methyl-3-methoxybutanol, propylene glycol t-butyl ether as well as mixtures of these solvents.

Solvents can be employed in the inventive liquid to gel-like detergents and cleaning compositions in amounts between 0.1 and 20 wt. %, preferably, however below 15 wt. % and particularly below 10 wt. %.

One or more thickeners or thickener systems can be added to the inventive compositions to adjust the viscosity. These high molecular weight substances, which are also called swelling agents, soak up mostly liquids, thereby swelling up and subsequently transform into viscous, real or colloidal solutions.

Suitable thickeners are inorganic or polymeric organic compounds. The inorganic thickeners include, for example, polysilicic acids, mineral clays like montmorillonite, zeolites, silicic acids and bentonites. The organic thickeners come from the groups of natural polymers, derivatives of natural polymers and synthetic polymers. Exemplary, naturally occurring polymers that can be used as thickeners are agar agar, carrageen, tragacanth, gum Arabic, alginates, pectins, polyoses, guar meal, locust tree bean flour, starches, dextrins, gelatines and casein. Modified natural products that are used as thickeners are mainly derived from the group of the modified starches and celluloses. Examples can be cited as carboxymethyl cellulose and other cellulose ethers, hydroxyethyl- and hydroxypropyl cellulose as well as bean flour ether. Totally synthetic thickeners are polymers such as polyacrylics and polymethacrylics, vinyl polymers, polycarboxylic acids, polyethers, polyimines, polyamides and polyurethanes.

The thickeners can be comprised in amounts up to 5 wt. %, preferably from 0.05 to 2 wt. %, and particularly preferably from 0.1 to 1.5 wt. %, based on the finished preparation.

The laundry detergents or cleaning compositions according to the invention can optionally comprise other conventional ingredients, such as sequestering agents, electrolytes and further auxiliaries.

The detergents for textiles may contain derivatives of diaminostilbene disulfonic acid or alkali metal salts thereof as optical brighteners. Suitable optical brighteners are, for example, salts of 4,4′-bis-(2-anilino-4-morpholino-1,3,5-triazinyl-6-)stilbene-2,2′-disulfonic acid or compounds of similar structure which contain a diethanolamino group, a methylamino group, an anilino group or a 2-methoxyethylamino group instead of the morpholino group. Optical brighteners of the substituted diphenylstyryl type may also be present, for example the alkali metal salts of 4,4′-bis(2-sulfostyryl)diphenyl, 4,4′-bis(4-chloro-3-sulfostyryl)diphenyl or 4-(4-chlorostyryl)-4′-(2-sulfostyryl)diphenyl. Mixtures of the mentioned optical brighteners may also be used.

Graying inhibitors have the task of ensuring that the dirt removed from the textile fibers is held suspended in the wash liquid. Water-soluble colloids of mostly organic nature are suitable for this, for example starch, glue, gelatines, salts of ether carboxylic acids or ether sulfonic acids of starches or celluloses, or salts of acidic sulfuric acid esters of celluloses or starches. Water-soluble, acid group-containing polyamides are also suitable for this purpose. Moreover, aldehyde starches, for example, can be used instead of the abovementioned starch derivatives. Preference, however, is given to the use of cellulose ethers such as carboxymethyl cellulose (Na salt), methyl cellulose, hydroxyalkyl cellulose, and mixed ethers such as methyl hydroxyethyl cellulose, methyl hydroxypropyl cellulose, methyl carboxymethyl cellulose and mixtures thereof, which can be added, for example in amounts of 0.1 to 5 wt. %, based on the composition.

In order to realize a silver corrosion protection, silver protectors for tableware can be added to the inventive cleaning compositions. Benzotriazoles, ferric chloride or CoSO₄, for example, are known from the prior art. As is known from the European Patent EP 0 736 084 B1, for example, particularly suitable silver corrosion inhibitors for general use with enzymes are salts and/or complexes of manganese, titanium, zirconium, hafnium, vanadium, cobalt or cerium, in which the cited metals exist in the valence states II, III, IV, V or VI.

Examples of these types of compounds are MnSO₄, V₂O₅, V₂O₄, VO₂, TiOSO₄, K₂TiF₆, K₂ZrF₆, Co(NO₃)₂, Co(NO₃)₃ and mixtures thereof.

Soil repellents are mostly polymers that when used in a detergent, lend the fibers soil-repelling properties and/or support the soil repellent capabilities of the conventional ingredients. A comparable effect can also be observed when they are added in cleaning compositions for hard surfaces.

Particularly effective and well-known soil release agents are copolyesters with dicarboxylic acid, alkylene glycol and polyalkylene glycol units. Examples of these are copolymers or mixed polymers of polyethylene terephthalate and polyoxyethylene glycol (DT 16 17 141 and DT 22 00 911). German Offenlegungsschrift DT 22 53 063 cites acidic compositions, which inter alia comprise a copolymer of a dibasic acid and an alkylene or cycloalkylene polyglycol. Polymers of ethylene terephthalate and polyethylene oxide terephthalate and their use in laundry detergents are described in the German texts DE 28 57 292 and DE 33 24 258 and the European Patent EP 0 253 567. The European Patent EP 066 944 relates to compositions, which contain a copolyester of ethylene glycol, polyethylene glycol, aromatic dicarboxylic acids and sulfonated aromatic dicarboxylic acids in defined molar ratios. Polyesters, end-capped with methyl or ethyl groups, with ethylene- and/or propylene terephthalate units and polyethylene oxide terephthalate units, and laundry detergents that comprise such a soil-release polymer are known from EP 0 185 427. The European Patent EP 0 241 984 relates to a polyester, which in addition to oxyethylene groups and terephthalic acid units also comprises substituted ethylene units as well as glycerine units. Polyesters are known from EP 0 241 985 which contain, beside oxyethylene groups and terephthalic acid units, 1,2-propylene-, 1,2-butylene- and/or 3-methoxy-1,2-propylene groups as well as glycerine units, and are end-capped with C₁ to C₄ alkyl groups. Polyesters with polypropylene terephthalate units and polyoxyethylene terephthalate units, at least partially end-capped with C₁₋₄ alkyl or acyl groups, are known from the European Patent application EP 0 272 033. The European Patent EP 0 274 907 describes soil-release polyesters containing terephthalate end-capped with sulfoethyl groups. According to the European Patent application EP 0 357 280, soil-release polyesters with terephthalate units, alkylene glycol units and poly-C₂₋₄ glycol units are manufactured by sulfonation of the unsaturated end groups. The international patent application WO 95/32232 relates to acidic, aromatic polyesters that are capable of releasing soil. For cotton materials, non-polymeric soil repellent active substances with a plurality of functional units are known from the international patent application WO 97/31085: a first unit, which can be cationic, for example, is able to be adsorbed onto the cotton surface by electrostatic attraction, and a second unit, which is designed to be hydrophobic, is responsible for the retention of the active agent at the water/cotton interface.

Color transfer inhibitors that can be used in inventive detergents for textiles particularly include polyvinyl pyrrolidones, polyvinyl imidazoles, polymeric N-oxides such as polyvinyl pyridine-N-oxide and copolymers of vinyl pyrrolidone with vinyl imidazole.

On using the agents in automatic cleaning processes, it can be advantageous to add foam inhibitors. Suitable foam inhibitors include for example, soaps of natural or synthetic origin, which have a high content of C₁₈-C₂₄ fatty acids. Suitable non-surface-active types of foam inhibitors are, for example, organopolysiloxanes and mixtures thereof with microfine, optionally silanized silica and also paraffins, waxes, microcrystalline waxes and mixtures thereof with silanized silica or bis-stearyl ethylene diamide. Mixtures of various foam inhibitors, for example mixtures of silicones, paraffins or waxes, are also used with advantage. Preferably, the foam inhibitors, especially silicone-containing and/or paraffin-containing foam inhibitors, are loaded onto a granular, water-soluble or dispersible carrier material. Especially in this case, mixtures of paraffins and bis stearylethylene diamides are preferred.

An inventive cleaning composition for hard surfaces can moreover comprise abrasive ingredients, especially from the group comprising quartz meal, wood flour, plastic powder, chalk and microspheres as well as their mixtures. Abrasives are preferably comprised in the inventive cleaning compositions in amounts of not more than 20 wt. %, particularly from 5 wt. % to 15 wt. %.

Colorants and fragrances may be added to the laundry detergents and cleaning compositions in order to improve the esthetic impression created by the products and to provide the consumer not only with the required performance but also with a visually and sensorially “typical and unmistakable” product. Suitable perfume oils or fragrances include individual odoriferous compounds, for example synthetic products of the ester, ether, aldehyde, ketone, alcohol and hydrocarbon type. Odoriferous compounds of the ester type are, for example, benzyl acetate, phenoxyethyl isobutyrate, p-tert.-butylcyclohexyl acetate, linalyl acetate, dimethylbenzyl carbinyl acetate, phenylethyl acetate, linalyl benzoate, benzyl formate, ethylmethylphenyl glycinate, allylcyclohexyl propionate, styrallyl propionate and benzyl salicylate. The ethers include, for example, benzyl ethyl ether; the aldehydes include, for example, the linear alkanals containing 8 to 18 carbon atoms, citral, citronellal, citronellyloxyacetaldehyde, cyclamen aldehyde, hydroxycitronellal, lilial and bourgeonal; the ketones include, for example, the ionones, α-isomethyl ionone and methyl cedryl ketone; the alcohols include anethol, citronellol, eugenol, geraniol, linalool, phenylethyl alcohol and terpineol and the hydrocarbons include, above all, the terpenes, such as limonene and pinene. However, mixtures of various odoriferous substances, which together produce an attractive fragrant note, are preferably used. Perfume oils such as these may also contain natural odoriferous mixtures obtainable from vegetal sources, for example pine, citrus, jasmine, patchouli, rose or ylang-ylang oil. Also suitable are muscatel oil, oil of sage, chamomile oil, clove oil, melissa oil, mint oil, cinnamon leaf oil, lime blossom oil, juniper berry oil, vetivert oil, olibanum oil, galbanum oil and laudanum oil and orange blossom oil, neroli oil, orange peel oil and sandalwood oil. Normally the content of dyes lies below 0.01 wt. %, while fragrances can make up to 2 wt. % of the total formulation of the laundry detergent and cleaning compositions.

The fragrances may be directly incorporated in the laundry detergent and cleaning compositions, although it can also be of advantage to apply the fragrances on carriers, which reinforce the adsorption of the perfume on the washing and thereby ensuring a long-lasting fragrance on the textiles by decreasing the release of the fragrance, especially for treated textiles. Suitable carrier materials are, for example, cyclodextrins, the cyclodextrin/perfume complexes optionally being coated with other auxiliaries. A further preferred carrier for fragrances is the described zeolite X, which instead of or in mixtures with surfactants can also take up fragrances. Accordingly, preferred laundry detergents and cleaning compositions comprise the described zeolite X and fragrances that are preferably at least partially absorbed on the zeolite.

Preferred colorants, which are not difficult for the expert to choose, have high storage stability, are not affected by the other ingredients of the composition or by light and do not have any pronounced substantivity for the textile fibers being treated, so as not to color them.

To control microorganisms, the laundry detergent or cleaning compositions may contain antimicrobial agents. Depending on the antimicrobial spectrum and the action mechanism, antimicrobial agents are classified as bacteriostatic agents and bactericides, fungistatic agents and fungicides, etc. Important substances from these groups are for example benzalkonium chlorides, alkylaryl sulfonates, halophenols and phenol mercury acetate. In the present context of the inventive teaching, the expressions “antimicrobial activity” and “antimicrobial agent” have the usual technical meaning as defined, for example, by K. H. Wallhäuβer in “Praxis der Sterilisation, Desinfektion—Konservierung Keimidentifizierung—Betriebshygiene” (5th Edition, Stuttgart/New York: Thieme, 1995), any of the substances with antimicrobial activity described therein being usable. Suitable antimicrobial agents are preferably selected from the groups of alcohols, amines, aldehydes, antimicrobial acids and salts thereof, carboxylic acid esters, acid amides, phenols, phenol derivatives, diphenyls, diphenylalkanes, urea derivatives, oxygen and nitrogen acetals and formals, benzamidines, isothiazolines, phthalimide derivatives, pyridine derivatives, antimicrobial surface-active compounds, guanidines, antimicrobial amphoteric compounds, quinolines, 1,2-dibromo-2,4-dicyanobutane, iodo-2-propyl butyl carbamate, iodine, iodophores, peroxy compounds, halogen compounds and mixtures of the above.

The antimicrobials can be selected from ethanol, n-propanol, i-propanol, 1,3-butanediol, phenoxyethanol, 1,2-propylenelycol, glycerin, undecylenic acid, benzoic acid, salicylic acid, dihydracetic acid, o-phenylphenol, N-methylmorpholine-acetonitrile (MMA), 2-benzyl-4-chlorophenol, 2,2′-methylene-bis-(6-bromo-4-chlorophenol), 4,4′-dichloro-2′-hydroxydiphenyl ether (dichlosan), 2,4,4′-trichloro-2′-hydroxydiphenyl ether (trichlosan), chlorhexidine, N-(4-chlorophenyl)-N-(3,4-dichlorophenyl)-urea, N,N′-(1,10-decanediyldi-1-pyridinyl-4-ylidene)-bis-(1-octamine) dihydrochloride, N,N′-bis-(4-chlorophenyl)-3,12-diimino-2,4,11,13-tetraaza-tetradecanediimideamide, glucoprotamines, surface-active antimicrobial quaternary compounds, guanidines, including the bi- and polyguanidines, such as for example 1,6-bis(2-ethylhexylbiguanidohexane) dihydrochloride, 1,6-di-(N₁,N₁′-phenyldiguanido-N₅,N₅′)hexane tetrahydrochloride, 1,6-di-(N₁,N₁-phenyl-N₁,N₁-methyldiguanido-N₅,N₅′)hexane dihydrochloride, 1,6-di-(N₁,N₁,N₁′-o-chlorophenyldiguanido-N₅,N₅′)hexane dihydrochloride, 1,6-di-(N₁,N₁′-2,6-dichlorophenyldiguanido-N₅,N₅′)hexane dihydrochloride, 1,6-di-[N₁,N₁′-β-(p-methoxyphenyl) diguanido-N₅,N₅′]hexane dihydrochloride, 1,6-di-(N₁,N₁′-α-methyl-β-phenyldiguanido-N₅,N₅′)hexane dihydrochloride, 1,6-di-(N₁,N₁′-p-nitrophenyldiguanido-N₅,N₅′)hexane dihydrochloride, phenyldiguanido-N₅,N₅′)di-n-propyl ether dihydrochloride, ω:ω-di-(N₁,N₁′-p-chlorophenyldiguanido-N₅,N₅′)di-n-propyl ether tetrahydrochloride, 1,6-di-(N₁,N₁′-2,4-dichlorophenyldiguanido-N₅,N₅′)hexane tetrahydrochloride, 1,6-di-(N₁,N₁′-p-methylphenyldiguanido-N₅,N₅′)hexane dihydrochloride, 1,6-di-[N₁,N₁′-2,4,5-trichlorophenyldiguanido-N₅,N₅′)hexane tetrahydrochloride, chlorophenyl)ethyldiguanido-N₅,N₅′]hexane dihydrochloride, ω:ω-di-(N₁,N₁,N₁′-p-chlorophenyldiguanido-N₅,N₅′)xylene dihydrochloride, 1,12-di-(N₁,N₁′-p-chlorophenyldiguanido-N₅,N₅′)dodecane dihydrochloride, 1,10-di-(N₁,N₁′-phenyldiguanido-N₅,N₅′)decane tetrahydrochloride, 1,12-di-(N₁,N₁′-phenyldiguanido-N₅,N₅′)dodecane tetrahydrochloride, 1,6-di-(N₁,N₁′-o-chlorophenyldiguanido-N₅,N₅′)hexane dihydrochloride, 1,6-di-(N₁,N₁′-o-chlorophenyldiguanido-N₅,N₅′)hexane tetrahydrochloride, ethylene-bis-(1-tolylphenylbiguanide), ethylene-bis-(p-tolylphenylbiguanide), ethylene-bis-(3,5-dimethylphenylbiguanide), ethylene-bis-(p-tert-amylphenylbiguanide), ethylene-bis-(nonylphenylbiguanide), ethylene-bis-(phenylbiguanide), ethylene-bis-(N-butylphenylbiguanide), ethylene-bis-(2,5-diethoxyphenylbiguanide), ethylene-bis-(2,4-dimethylphenylbiguanide), ethylene-bis-(o-diphenylbiguanide), ethylene-bis-(mixed amylnaphthylbiguanide), N-butylethylene-bis-(phenylbiguanide), trimethylene bis(o-tolylbiguanide), N-butyltrimethylene-bis-(phenylbiguanide) and the corresponding salts like acetates, gluconates, hydrochlorides, hydrobromides, citrates, bisulfites, fluorides, polymaleates, N-coco alkyl sarcinosates, phosphites, hypophosphites, perfluorooctanoates, silicates, sorbates, salicylates, maleates, tartrates, fumarates, ethylenediaminetetraacetates, iminodiacetates, cinnamates, thiocyanates, arginates, pyromellitates, tetracarboxybutyrates, benzoates, glutarates, monofluorophosphates, perfluoropropionates as well as any mixtures thereof. Furthermore, halogenated xylene- and cresol derivatives are suitable, such as p-chloro-meta-cresol, p-chloro-meta-xylene, as well as natural antimicrobial active agents of plant origin (e.g. from spices or aromatics), animal as well as microbial origin. Preferred antimicrobial agents are antimicrobial surface-active quaternary compounds, a natural antimicrobial agent of vegetal origin and/or a natural antimicrobial agent of animal origin and, most preferably, at least one natural antimicrobial agent of vegetal origin from the group comprising caffeine, theobromine and theophylline and essential oils, such as eugenol, thymol and geraniol, and/or at least one natural antimicrobial agent of animal origin from the group comprising enzymes, such as protein from milk, lysozyme and lactoperoxidase and/or at least one antimicrobial surface-active quaternary compound containing an ammonium, sulfonium, phosphonium, iodonium or arsonium group, peroxy compounds and chlorine compounds. Substances of microbial origin, so-called bacteriozines, may also be used.

The quaternary ammonium compounds (QUATS) suitable as antimicrobial agents have the general formula (R¹)(R²)(R³)(R⁴)N⁺X⁻, in which R¹ to R⁴ may be the same or different and represent C₁₋₂₂ alkyl groups, C₇₋₂₅ aralkyl groups or heterocyclic groups, two or—in the case of an aromatic compound, such as pyridine—even three groups together with the nitrogen atom forming the heterocycle, for example a pyridinium or imidazolinium compound, and X⁻ represents halide ions, sulfate ions, hydroxide ions or similar anions. In the interests of optimal antimicrobial activity, at least one of the substituents preferably has a chain length of 8 to 18 and, more preferably, 12 to 16 carbon atoms.

QUATS can be obtained by reacting tertiary amines with alkylating agents such as, for example, methyl chloride, benzyl chloride, dimethyl sulfate, dodecyl bromide and also ethylene oxide. The alkylation of tertiary amines having one long alkyl chain and two methyl groups is particularly easy. The quaternization of tertiary amines containing two long chains and one methyl group can also be carried out under mild conditions using methyl chloride. Amines containing three long alkyl chains or hydroxy-substituted alkyl chains lack reactivity and are preferably quaternized with dimethyl sulfate.

Suitable QUATS are, for example, Benzalkonium chloride (N-alkyl-N,N-dimethylbenzyl ammonium chloride, CAS No. 8001-54-5), Benzalkon B (m,p-dichlorobenzyl dimethyl-C₁₋₂-alkyl ammonium chloride, CAS No. 58390-78-6), Benzoxonium chloride (benzyldodecyl-bis-(2-hydroxyethyl) ammonium chloride), Cetrimonium bromide (N-hexadecyl-N,N-trimethyl ammonium bromide, CAS No. 57-09-0), Benzetonium chloride (N,N-di-methyl-N-[2-[2-[p-(1,1,3,3-tetramethylbutyl)-phenoxy]-ethoxy]-ethyl]-benzyl ammonium chloride, CAS No. 121-54-0), dialkyl dimethyl ammonium chlorides, such as di-n-decyldimethyl ammonium chloride (CAS No. 7173-51-5-5), didecyldimethyl ammonium bromide (CAS No. 2390-68-3), dioctyl dimethyl ammonium chloride, 1-cetylpyridinium chloride (CAS No. 123-03-5) and thiazoline iodide (CAS No. 1576448-1) and mixtures thereof. Particularly preferred QUATS are the benzalkonium chlorides containing C₈₋₁₈ alkyl groups, more particularly C₁₂-C₁₄ alkylbenzyl dimethylammonium chloride.

Benzalkonium halides and/or substituted benzalkonium halides are commercially available, for example, as Barquat® from Lonza, Marquato® from Mason, Variquat® from Witco/Sherex and Hyamine from Lonza and as Bardac® from Lonza.

Other commercially obtainable antimicrobial agents are N-(3-chloroallyl)-hexaminium chloride, such as Dowicide® and Dowicil® from Dow, benzethonium chloride, such as Hyamine® 1622 from Rohm & Haas, methyl benzethonium chloride, such as Hyamine® 10× from Rohm & Haas, cetyl pyridinium chloride, such as cepacolchloride from Merrell Labs.

The antimicrobial agents are used in quantities of 0.0001% by weight to 1% by weight, preferably 0.001% by weight to 0.8% by weight, particularly preferably 0.005% by weight to 0.3% by weight and most preferably 0.01 to 0.2% by weight.

The compositions may comprise UV absorbers, which attach to the treated textiles and improve the light stability of the fibers and/or the light stability of the various ingredients of the formulation. UV-absorbers are understood to mean organic compounds, which are able to absorb UV radiation and emit the resulting energy in the form of longer wavelength radiation, for example as heat.

Compounds, which possess these desired properties, are for example, the efficient radiationless deactivating derivatives of benzophenone having substituents in position(s) 2 and/or 4. Also suitable are substituted benzotriazoles, acrylates, which are phenyl-substituted in position 3 (cinnamic acid derivatives optionally with cyano groups in position 2), salicylates, organic Ni complexes, as well as natural substances such as umbelliferone and the endogenous urocanic acid. The biphenyl and above all the stilbene derivatives such as for example those described in EP 0 728 749 A and commercially available as Tinosore FD or Tinosorb® FR from Ciba, are of particular importance. As UV-B absorbers can be cited: 3-benzylidenecamphor or 3-benzylidenenorcamphor and its derivatives, for example 3-(4-methylbenzylidene) camphor, as described in the EP 0693471 B1; 4-aminobenzoic acid derivatives, preferably 4-(dimethylamino)benzoic acid, 2-ethylhexyl ester, 4-(dimethylamino)benzoic acid, 2-octyl ester and 4-(dimethylamino)benzoic acid, amyl ester; esters of cinnamic acid, preferably 4-methoxycinnamic acid, 2-ethylhexyl ester, 4-methoxycinnamic acid, propyl ester, 4-methoxycinnamic acid, isoamyl ester, 2-cyano-3,3-phenylcinnamic acid, 2-ethylhexyl ester (octocrylene); esters of salicylic acid, preferably salicylic acid, 2-ethylhexyl ester, salicylic acid, 4-isopropylbenzyl ester, salicylic acid, homomethyl ester; derivatives of benzophenone, preferably 2-hydroxy-4-methoxybenzophenone, 2-hydroxy-4-methoxy-4′-methylbenzophenone, 2,2′-dihydroxy-4-methoxybenzophenone; esters of benzalmalonic acid, preferably 4-methoxybenzmalonic acid, di-2-ethylhexylester; triazine derivatives, such as, for example 2,4,6-trianilino-(p-carbo-2′-ethyl-1′-hexyloxy)-1,3,5-triazine and octyl triazone, as described in EP 0818450 A1 or dioctyl butamidotriazone (Uvasorb® HEB); propane-1,3-dione, such as for example 1-(4-tert.-butylphenyl)-3-(4′-methoxyphenyl)propane-1,3-dione; ketotricyclo(5.2.1.0) decane derivatives, as described in EP 0694521 B1. Further suitable compounds are 2-phenylbenzimidazole-5-sulfonic acid and its alkali-, alkaline earth-, ammonium-, alkylammonium-, alkanolammonium- and glucammonium salts; sulfonic acid derivatives of benzophenones, preferably 2-hydroxy-4-methoxybenzophenone-5-sulfonic acid and its salts; sulfonic acid derivatives of 3-benzylidenecamphor, as for example 4-(2-oxo-3-bornylidenemethyl)benzene sulfonic acid and 2-methyl-5-(2-oxo-3-bornylidene) sulfonic acid and its salts.

Typical UV-A filters particularly include derivatives of benzoylmethane, such as, for example 1-(4′-tert.-butylphenyl)-3-(4′-methoxyphenyl)propane-1,3-dione, 4-tert.-butyl-4′-methoxydibenzoylmethane (Parsol 1789), 1-phenyl-3-(4′-isopropylphenyl)-propane-1,3-dione as well as enamine compounds, as described in the DE 1 971 2033 A1 (BASF). Naturally, the UV-A and UV-B filters can also be added as mixtures. Beside the cited soluble materials, insoluble, light protective pigments, namely finely dispersed, preferably, nano metal oxides or salts can also be considered for this task. Exemplary suitable metal oxides are particularly zinc oxide and titanium oxide and also oxides of iron, zirconium, silicon, manganese, aluminum and cerium as well as their mixtures. Silicates (talc), barium sulfate or zinc stearate can be added as salts. The oxides and salts are already used in the form of pigments for skin care and skin protecting emulsions and decorative cosmetics. Here, the particles should have a mean diameter of less than 100 nm, preferably between 5 and 50 nm and especially between 15 and 30 nm. They can be spherical, however elliptical or other shaped particles can also be used. The pigments can also be surface treated, i.e. hydrophilized or hydrophobized. Typical examples are coated titanium dioxides, such as, for example Titandioxid Z 805 (Degussa) or Eusolex® T2000 (Merck); preferably, silicones and particularly preferably trialkoxy octylsilanes or Simethicones are used as the hydrophobic coating agents Preferably, micronized zinc oxide is used. Further suitable UV light protection filters may be found in the review by P. Finkel in SöFW-Journal, Volume 122 (1996), p. 543.

The UV absorbers are normally used in amounts of 0.01 wt. % to 5 wt. %, preferably from 0.03 wt. % to 1 wt. %.

To increase their washing or cleaning power, compositions according to the invention can comprise, in addition to the inventive enzymes, additional enzymes, in principle any enzyme established for these purposes in the prior art being useable. These particularly include proteases, amylases, lipases, hemicellulases, cellulases or oxidoreductases as well as preferably their mixtures. In principle, these enzymes are of natural origin; improved variants based on the natural molecules are available for use in detergents and accordingly they are preferred. The agents according to the invention preferably comprise enzymes in total quantities of 1×10⁻⁶ to 5 weight percent based on active protein. The protein concentration can be determined using known methods, for example the BCA Process (bicinchoninic acid; 2,2′-biquinolyl-4,4′-dicarboxylic acid) or the biuret process (A. G. Gornall, C. S. Bardawill and M. M. David, J. Biol. Chem., 177 (1948), pp. 751-766).

Preferred proteases are those of the subtilisin type. Examples of these are subtilisins BPN′ and Carlsberg, the protease PB92, the subtilisins 147 and 309, the alkaline protease from Bacillus lentus, subtilisin DY and those enzymes of the subtilases no longer however classified in the stricter sense as subtilisins, thermitase, proteinase K and the proteases TW3 and TW7. Subtilisin Carlsberg in further developed form is available under the trade name Alcalase® from Novozymes A/S, Bagsvrd, Denmark. Subtilisins 147 and 309 are commercialized under the trade names Esperase® and Savinase® by the Novozymes company. Variants derived from the protease from Bacillus lentus DSM 5483 (WO 91/02792 A1) called BLAP® are described especially in WO 92/21760 A1, WO 95/23221 A1, WO 02/088340 A2 and WO 03/038082 A2. Further useable proteases from various Bacillus sp. and B. gibsonii emerge from the patent applications WO 03/054185 A1, WO 03/056017 A2, WO 03/055974 A2 and WO 03/054184 A1.

Further useable proteases are, for example, those enzymes available with the trade names Durazym®, Relase®, Everlase®, Nafizym, Natalase®, Kannase® and Ovozymes® from the Novozymes Company, those under the trade names Purafect®, Purafect® OxP and Properase® from Genencor, that under the trade name Protosol® from Advanced Biochemicals Ltd., Thane, India, that under the trade name Wuxi® from Wuxi Snyder Bioproducts Ltd., China, those under the trade names Proleather® and Protease P® from Amano Pharmaceuticals Ltd., Nagoya, Japan, and that under the designation Proteinase K-16 from Kao Corp., Tokyo, Japan.

Examples of further useable amylases according to the invention are the α-amylases from Bacillus licheniformis, from B. amyloliquefaciens and from B. stearothermophilus, as well as their improved further developments for use in laundry detergents and cleaning compositions. The enzyme from B. licheniformis is available from the Novozymes Company under the name Termamyl® and from the Genencor Company under the name Purastar®ST. Further development products of this α-amylase are available from the Novozymes Company under the trade names Duramyl® and Termamyl®ultra, from the Genencor Company under the name Purastar® OxAm and from Daiwa Seiko Inc., Tokyo, Japan as Keistase®. The α-amylase from B. amyloliquefaciens is commercialized by the Novozymes Company under the name BAN®, and derived variants from the α-amylase from B. stearothermophilus under the names BSG® and Novamyl® also from the Novozymes Company. Moreover, for these purposes, attention should be drawn to the α-amylase from Bacillus sp. A 7-7 (DSM 12368) disclosed in the application WO 02/10356 A2 and the cyclodextrin-glucanotransferase (CGTase) from B. agaradherens (DSM 9948) described in the application WO 02/44350 A2. Furthermore, the amylolytic enzymes are useable, which belong to the sequence space of α-amylase, described in the application WO 03/002711 A2 and those described in the application WO 03/054177 A2. Similarly, fusion products of the cited molecules are applicable, for example those from the application DE 10138753 A1 or their point mutations. Moreover, further developments of α-amylase from Aspergillus niger and A. oryzae available from the Company Novozymes under the trade name Fungamyl® are suitable. Additional commercial products that can be used are for example the Amylase-LT® and Stainzyme®, the latter also from the Novozymes company. Moreover, for these purposes, attention should be drawn to the α-amylase from Bacillus sp. A 7-7 (DSM 12368) disclosed in the application WO 02/10356 A2 and the cyclodextrin-glucanotransferase (CGTase) from B. agaradherens (DSM 9948) described in the application WO 02/44350 A2. Furthermore, the amylolytic enzymes are useable, which belong to the sequence space of α-amylase, described in the application WO 03/002711 A2 and those described in the application WO 03/054177 A2. Moreover, further developments of α-amylase from Aspergillus niger and A. oryzae available from the Company Novozymes under the trade name Fungamyl® are suitable. A further commercial product is the amylase-LT® for example.

Compositions according to the invention, particularly when they are destined for treating textiles, can comprise cellulases, according to their purpose, as pure enzymes, as enzyme preparations, or in the form of mixtures in which the individual components advantageously complement their various performances. Among these aspects of performance are particular contributions to primary washing performance, to secondary washing performance of the product, (anti-redeposition activity or inhibition of graying) and softening or brightening (effect on the textile), through to practicing a “stone washed” effect.

A usable, fungal endoglucanase (EG)-rich cellulase preparation, or its further developments, are offered by the Novozymes Company under the trade name Celluzyme®. The products Endolase® and Carezyme® based on the 50 kD-EG, respectively 43 kD-EG from H. insolens DSM 1800 are also obtainable from Novozymes Company. The latter is based on the application WO 96/29397 A1. Performance enhanced cellulase variants emerge from the application WO 98/12307 A1, for example. It is equally possible to use the cellulases disclosed in the application WO 97/14804 A1; for example the 20 kD EG disclosed therein from Melanocarpus, and which is available under the trade names Ecostone® and Biotouch® from AB Enzymes, Finland. Further commercial products from the AB Enzymes Company are Econase® and Ecopulp®. Further suitable cellulases from Bacillus sp. CBS 670.93 and CBS 669.93 are disclosed in WO 96/34092 A2, the CBS 670.93 from Bacillus sp. being obtainable under the trade name Puradax® from the Genencor Company. Other commercial products from the Genencor Company are “Genencor detergent cellulase L” and IndiAge® Neutra.

The compositions according to the invention can comprise additional enzymes especially for removing specific problem stains and which are summarized under the term hemicellulases. These include, for example mannanases, xanthanlyases, pectinlyases (=pectinases), pectinesterases, pectatlyases, xyloglucanases (=xylanases), pullulanases and β-glucanases. Suitable mannanases, for example are available under the names Gamanase® and Pektinex AR® from Novozymes Company, under the names Rohapec® B1L from AB Enzymes and under the names Pyrolase® from Diversa Corp., San Diego, Calif., USA. A suitable β-Glucanase from a B. alcalophilus emerges from the application WO 99/06573 A1, for example. β-Glucanase extracted from B. subtilis is available under the name Cereflo® from Novozymes Company.

To increase the bleaching action, the laundry detergent and cleaning compositions according to the invention can comprise oxidoreductases, for example oxidases, oxygenases, katalases, peroxidases, like halo-, chloro-, bromo-, lignin-, glucose- or manganese-peroxidases, dioxygenases or laccases (phenoloxidases, polyphenoloxidases) as well as perhydrolases, bleach activators or bleach catalysts. Suitable commercial products are Denilite® 1 and 2 from the Novozymes Company. Advantageously, additional, preferably organic, particularly preferably aromatic compounds are added that interact with the enzymes to enhance the activity of the oxidoreductases in question or to facilitate the electron flow (mediators) between the oxidizing enzymes and the stains over strongly different redox potentials.

The enzymes additionally used in the compositions according to the invention either stem originally from microorganisms, such as the genera Bacillus, Streptomyces, Humicola, or Pseudomonas, and/or are produced according to known biotechnological processes using suitable microorganisms such as by transgenic expression hosts of the genera Escherichia coli, Bacillus, Streptomyces or filamentary fungi.

Purification of the relevant enzymes follows conveniently using established processes such as precipitation, sedimentation, concentration, filtration of the liquid phases, microfiltration, ultrafiltration, mixing with chemicals, deodorization or suitable combinations of these steps.

The enzymes—the amadoriases as well as the optionally additionally employed enzymes—can be added to the inventive compositions in each established form known from the prior art. Included here, for example, are solid preparations obtained by granulation, extrusion or lyophilization, or particularly for liquid compositions or compositions in the form of gels, enzyme solutions, advantageously highly concentrated, of low moisture content and/or mixed with stabilizers.

Alternatively, both for the solid as well as the liquid presentation form, the enzymes can also be encapsulated, for example by spray drying or extrusion of the enzyme solution together with a preferably natural polymer or in the form of capsules, for example those in which the enzyme is embedded in a solidified gel, or in those of the core-shell type, in which an enzyme-containing core is covered with a water-, air- and/or chemical-impervious protective layer. Further active principles, for example stabilizers, emulsifiers, pigments, bleaches or colorants can be applied in additional layers. Such capsules are made using known methods, for example by vibratory granulation or roll compaction or by fluidized bed processes. Advantageously, these types of granulates, for example with an applied polymeric film former are dust-free and as a result of the coating are storage stable.

In addition, it is possible to formulate two or more enzymes together, such that a single granulate exhibits a plurality of enzymatic activities.

The protein concentration on the comprised enzymes, particularly on the comprised choline oxidases can be determined using known methods, for example the BCA Process (bicinchoninic acid; 2,2′-biquinolyl-4,4′-dicarboxylic acid) or the biuret process (A. G. Gornall, C. S. Bardawill and M. M. David, J. Biol. Chem., 177 (1948), pp. 751-766).

A protein and/or enzyme in an inventive composition can be protected, particularly in storage, against deterioration such as, for example inactivation, denaturation or decomposition, for example through physical influences, oxidation or proteolytic cleavage. An inhibition of the proteolysis is particularly preferred during microbial preparation of proteins and/or enzymes, particularly when the compositions also contain proteases. Preferred compositions according to the invention comprise stabilizers for this purpose.

One group of stabilizers are the reversible protease inhibitors. For this, benzamidine hydrochloride, borax, boric acids, boronic acids or their salts or esters are frequently used, above all derivatives with aromatic groups, for example ortho, meta or para substituted phenyl boronic acids, particularly 4-formylphenyl boronic acid or the salts or esters of the cited compounds. Peptide aldehydes, i.e. oligopeptides with a reduced C-terminus, particularly those from 2 to 50 monomers are also used for this purpose. Ovomucoid and leupeptin, among others, belong to the peptidic reversible protease inhibitors. Specific, reversible peptide inhibitors for the protease subtilisin and fusion proteins from proteases and specific peptide inhibitors are also suitable.

Further enzyme stabilizers are amino alcohols like mono-, di-, triethanol- and -propanolamine and their mixtures, aliphatic carboxylic acids up to C₁₂, such as, for example succinic acid, other dicarboxylic acids or salts of the cited acids. End-capped fatty acid amide alkoxylates are also suitable for this purpose. Certain organic acids used as builders can, as disclosed in WO 97/18287 additionally stabilize an included enzyme.

Lower aliphatic alcohols, but above all polyols such as, for example glycerine, ethylene glycol, propylene glycol or sorbitol are further frequently used enzyme stabilizers. Di-glycerol phosphate also protects against denaturation by physical influences. Similarly, calcium and/or magnesium salts are used, such as, for example calcium acetate or calcium formate.

Polyamide oligomers or polymeric compounds like lignin, water-soluble vinyl copolymers or cellulose ethers, acrylic polymers and/or polyamides stabilize enzyme preparations inter alia against physical influences or pH variations. Polymers containing polyamine-N-oxide act simultaneously as enzyme stabilizers and color transfer inhibitors. Other polymeric stabilizers are linear C₈-C₁₈ polyoxyalkylenes. Alkyl polyglycosides can also stabilize the enzymatic components of the inventive compositions and are additionally capable of advantageously increasing their performance. Crosslinked nitrogen-containing compounds chiefly perform a dual function as soil release agents and as enzyme stabilizers. A hydrophobic, non-ionic polymer stabilizes in particular an optionally present cellulase.

Reducing agents and antioxidants increase the stability of enzymes against oxidative decomposition; sulfur-containing reducing agents are commonly used here. Other examples are sodium sulfite and reducing sugars, but also katalases in suitably low concentration.

The use of combinations of stabilizers is particularly preferred, for example of polyols, boric acid and/or borax, the combination of boric acid or borate, reducing salts and succinic acid or other dicarboxylic acids or the combination of boric acid or borate with polyols or polyamino compounds and with reducing salts. The effect of peptide-aldehyde stabilizers is conveniently increased by the combination with boric acid and/or boric acid derivatives and polyols and still more by the additional effect of divalent cations, such as for example calcium ions.

In a preferred embodiment, inventive compositions are characterized in that they consist of more than one phase in order to liberate the comprised active principles separately from one another at different times or from different places, for example. This can concern phases in different aggregates, however it particularly concerns phases in the same aggregates.

The inventive compositions are preferably present a single or multi-phase, especially two-phase liquid and sprayable composition that can be applied especially in a bottle having a spray system. On spraying, the composition is particularly well saturated with oxygen, which is why one can speak of a particular advantage for an enzyme-bleach or disinfection.

The multi-phase liquid and sprayable compositions (sprays) are either applied with a multi-chamber bottle having a spray system or they are shaken several times before use to produce a temporary emulsion.

Suitable organic solvents for producing the two-phase nature of the composition are for example hydrocarbons and alkyl ethers. Particularly preferred hydrocarbons have a boiling point above 150° C. and preferably above 180° C. In particular, dialkyl ethers, principally C₆-C₁₈ alkyl ethers and particularly preferably the C₈-C₁₂ alkyl ethers, for example dioctyl ether, can be considered as the alkyl ethers.

Other organic solvents that also exhibit an excellent cleaning effect include butoxy propoxy propanols (BPP) that are commercially available as a mixture of several isomers. As BPPs are not completely miscible with water, they are particularly suitable for use in multi-phase compositions. If BPPs are intended to be used in single-phase aqueous-based compositions, then additional emulsifiers need to be added. Examples of possible emulsifiers can be found in the disclosure of WO 96/30580.

The inventive compositions can comprise surfactants, namely anionic, non-ionic, cationic, zwitterionic, amphoteric surfactants or any mixtures thereof. The surfactant content of the compositions can vary over a wide range. Even small quantities that still show a soil dispersing and hence enhanced cleaning action, can be comprised; the surfactant content of the composition is for example in the range of 0.2 to 10 wt. %, wherein contents of 0.5 to 10 wt. % and especially 1 to 8 wt. % of anionic and/or non-ionic surfactants is particularly preferred. Amphoteric surfactants and/or cationic surfactants are also preferred for use in the present invention and can additionally act as fabric softeners.

In addition, the compositions can comprise fragrances, deodorants, preservatives, antibacterials, insect repellents (for moth proofing) and/or colorants, such as one or a plurality of finishers, lubricants, fungicides etc., as long as these additives do not destroy the dispersion of the composition. Also, pH-regulating substances, such as for example organic acids, especially citric acid, succinic acid, glutaric acid, adipic acid, gluconic acid and any mixture thereof, can be comprised. The amounts of these additives include—when present—generally about 0.2 to 5 wt. % of the total composition.

The liquid and sprayable compositions are preferably used in a bottle having a spraying system. Bottles having a spraying system are known to the person skilled in the art from the prior art. Bottles having a pump or trigger spray container, which allow a fine and concentrated squirt of spray to be precisely applied onto the fabric, are particularly suitable. The bottles can be manufactured from any materials known for this purpose; usually they are polymer and plastic bottles, polyethylenes and polypropylenes being preferred. However, other materials that are likewise suitable are also known to the person skilled in the art. The bottle can possess a closure cap to protect the spray device. Here, embodiments from the prior art are also known to the person skilled in the art. Pumps are also suitable that when used produce foam. A foam producing device of this type is known in the market as the “F2 Finger Pump Foamer” from the Airspray® Company. The preferred spray dispenser is a manually operated spray dispenser, selected in particular from the group including aerosol spray dispensers, self generated pressure spray dispensers, pump spray dispensers and trigger spray dispensers, in particular pump spray dispensers and trigger spray dispensers with a container of polyethylene or polyethylene terephthalate. Such trigger bottles are offered, for example, by the Afa-Polytec Company. The spray head is preferably equipped with a foam nozzle. One-handle dispensers or one-arm dispensers, which include and enclose a liquid container and a foam production device, are also known to the person skilled in the art from the prior art. An improved embodiment of one of this type of foam dispenser is described in the German patent application 199 51 011.3.

Inventive compositions, which are composed of more than one solid component, can be easily manufactured by mixing together the various solid components in bulk form, particularly powders, granules or extrudates with various ingredients and/or with a different release behavior. The manufacture of a solid composition according to the invention with one or more phases can be made by known methods, for example by spray drying or granulation, wherein the enzymes and possible further heat-sensitive ingredients, such as, for example bleaching agent are optionally added separately. For manufacturing inventive compositions with an increased bulk density, particularly in the range of 650 g/l to 950 g/l, a preferred process is one with an extrusion step, known from the European Patent EP 0 0486 592. A further preferred manufacturing using a granulation process is described in the European Patent EP 0 642 576.

For solid compositions, proteins can be used, for example, in dried, granulated, encapsulated or encapsulated and additionally dried form. They can be added separately, i.e. as one phase, or together with other ingredients in the same phase, with or without compaction. If microencapsulated, solid enzymes are used, then the water can be removed from the aqueous solutions resulting from the process by means of processes known from the prior art, such as spray-drying, centrifugation or by transdissolution. The particles obtained in this manner normally have a particle size between 50 and 200 μm.

The encapsulated form also serves, as previously discussed, to protect the enzymes from other ingredients such as bleaching agents, or to enable a controlled release. Depending on their size, said capsules are divided into micro- and nanocapsules, microcapsules being particularly preferred for enzymes. Such capsules are disclosed, for example, in the Patent applications WO 97/24177 and DE 199 18 267. Another possible encapsulation method consists in the encapsulation of the enzymes, suitable for laundry detergent or cleaning compositions, in starch or in a starch derivative, starting from a mixture of the enzyme solution with a solution or suspension of starch or a starch derivative. Such an encapsulation process is described in the German application DE 199 56 382.

At least two solid phases can also be combined with each other. Thus, it is possible to prepare a solid composition according to the invention by compression or compaction into tablets. Such tablets can be single-phase or multi-phase tablets. Consequently, this presentation form also offers the possibility of displaying a solid inventive composition having two solid phases. For manufacturing the inventive compositions in tablet form, which can be monophasic or multiphasic, single colored or multicolored and/or consisting of one or more layers, all the ingredients—optionally for each layer—are preferably mixed together in a mixer and the mixture is compressed using conventional tablet presses, e.g. exocentric presses or rotating presses with compression forces in the range of about 50 to 100 kN/cm², preferably 60 to 70 kN/cm². Particularly for the case of multilayer tablets, it can be advantageous to precompress at least one layer. This is preferably carried out using compression forces between 5 and 20 kN/cm², particularly 10 to 15 kN/cm². Tablets prepared in this way preferably have a weight of 10 g to 50 g, particularly 15 g to 40 g. The tablets may be any shape—round, oval or cornered—intermediate shapes also being possible.

It is particularly advantageous for multiphase compositions, that at least one of the phases comprises an amylase-sensitive material, especially starch, or is at least partially encapsulated or coated with this. In this way this phase is mechanically stabilized and/or protected against external influences and simultaneously attacked by an active amylase present in the wash liquor, such that the release of the ingredients is facilitated.

Similarly, preferred compositions according to the invention are characterized in that they are all in liquid, gel or paste form. The proteins, preferably a protein according to the invention, are added to such compositions and preferably result from a prior art protein extraction and preparation in concentrated aqueous or non-aqueous solution, for example in liquid form, such as solution, suspension or emulsion, but also in gel form or encapsulated or as dried powder. These types of inventive laundry detergent or cleaning composition in the form of solutions in standard solvents are generally prepared by a simple mixing of the ingredients, which can be added in the substance or as a solution into an automatic mixer.

An embodiment of the present invention is such a liquid, gel or paste agent, to which has been added an encapsulated protein essential for the invention and/or one of the other comprised proteins and/or one of the other comprised ingredients, preferably in the form of microcapsules. Among these, those encapsulated with amylase-sensitive materials are particularly preferred. The use of a combination of amylase-sensitive materials and an amylolytic enzyme in a laundry detergent or cleaning composition can demonstrate synergistic effects in such a way that the starch-cleaving enzyme supports the breakdown of the microcapsule and thereby controls the release process of the encapsulated ingredients with the result that their release does not happen during storage and/or not at the beginning of the cleaning process, but rather at a defined time. By this mechanism, complex laundry detergent and cleaning composition systems can be based on the most varied ingredients and the most varied capsule types, which represent the particularly preferred embodiments of the present invention.

A comparable effect is then given when the ingredients of the laundry detergent or cleaning composition are distributed in at least two different phases, for example two or more solid associated phases of a tableted laundry detergent or cleaning composition, or different granulates in the same powdery composition. Two-phase or multi-phase cleaners are state of the art for use in both automatic dishwashers as well as laundry detergents. The activity of an amylolytic enzyme in an earlier activated phase is a prerequisite for the activation of a later phase, when this is surrounded by an amylase-sensitive shell or coating, or the amylase-sensitive material represents an integral ingredient of the solid phase, whose partial or total hydrolysis disintegrates the relevant phase.

The ingredients of laundry detergent and cleaning compositions are capable of suitably supporting each other's performance. Thus, it is known from the application WO 98/45396, that polymers, which can be added as cobuilders, such as for example alkyl polyglycosides, can simultaneously stabilize and augment the activity and stability of included enzymes. Accordingly, it is preferred when a polyoloxidase according to the invention is modified by one of the customary ingredients mentioned above, is especially stabilized and/or its contribution to the performance of the laundry detergent or cleaning composition is increased.

Processes for cleaning textiles or hard surfaces constitute a further subject matter of the invention and are characterized in that an above-described inventive amadoriase is active in at least one of the process steps.

In this embodiment, the invention is realized in that the inventively improved enzymatic properties are utilized in principal in terms of an improvement in each cleaning process. Each cleaning process is enhanced in the relevant activity when it is added in at least one process step. Such processes are realized in machines such as standard household automatic dishwashers or household washing machines. Preferred processes are correspondingly preferred according to the abovementioned details. Further preferred processes are those wherein the amadoriase is incorporated through an above-described composition.

A further subject matter of the invention is a hair shampoo and/or hair care composition comprising inventively employable amadoriases.

The shampoos and/or hair care compositions as well as bubble baths, shower baths, creams, gels, lotions, alcoholic and aqueous-alcoholic solutions, emulsions, wax/fatty masses, sticks, powders or salves that include inventively useable amadoriases, can comprise mild surfactants, oils, emulsifiers, greases, pearlescent waxes, consistence providers, thickeners, polymers, silicone compounds, fats, waxes, stabilizers, biogenetic active principles, deodorants, anti-perspirants, anti-dandruff agents, film formers, swelling agents, UV-light protection factors, antioxidants, hydrotropes, preservatives, insect repellents, sun tans, solubilizers, perfume oils, colorants and the like as auxiliaries and additives.

Typical examples of suitable mild, i.e. particularly skin-compatible surfactants are fatty alcohol polyglycol ether sulfonates, monoglyceride sulfates, mono and/or dialkylsulfosuccinates, fatty acid isethionates, fatty acid sarcosinates, fatty acid taurides, fatty acid glutamates, α-olefin sulfonates, ether carboxylic acids, alkyl oligoglucosides, fatty acid glucamides, alkylamido betaines and/or protein-fatty acid condensates, the last preferably on the basis of wheat proteins.

The following can be considered as oils, for example: Guerbet alcohols based on fatty alcohols with 6 to 18, preferably 8 to 10 carbon atoms, esters of linear C₅-C₂₂-fatty acids with linear C₆-C₂₂ fatty alcohols, esters of branched C₆-C₁₃-carboxylic acids with linear C₆-C₂₂ fatty alcohols, such as for example myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. In addition, suitable esters are esters of linear C₆-C₂₂ fatty acids with branched alcohols, especially 2-ethylhexanol, esters of hydroxycarboxylic acids with linear or branched C₆-C₂₂ fatty alcohols, especially dioctyl malate, esters of linear and/or branched fatty acids with polyhydroxy alcohols (e.g. propylene glycol, dimerdiol or trimertriol) and/or Guerbet alcohols, triglycerides based on C₆-C₁₀ fatty acids, liquid mono-/di/triglyceride mixtures based on C₆-C₁₈ fatty acids, esters of C₆-C₂₂ fatty alcohols and/or Guerbet alcohols with aromatic carboxylic acids, especially benzoic acid, esters of C₂-C₁₂ dicarboxylic acids with linear or branched alcohols with 1 to 22 carbon atoms or polyols with 2 to 10 carbon atoms and 2 to 6 hydroxyl groups, vegetal oils, branched primary alcohols, substituted cyclohexanes, linear and branched C₆-C₂₂ fatty alcohol carbonates, Guerbet carbonates, esters of benzoic acid with linear and/or branched C₆-C₂₂ alcohols (e.g. Finsolv® TN), linear or branched, symmetrical or unsymmetrical dialkyl ethers with 6 to 22 carbon atoms per alkyl group, ring-opened products of epoxidized fatty acid esters with polyols, silicone oils and/or aliphatic or naphthenic hydrocarbons, such as, for example squalane, squalene or dialkylcyclohexanes.

Emulsifiers can be selected, for example, from non-ionic surfactants from at least one of the following groups:
(1) Addition products of 2 to 30 moles ethylene oxide and/or 0 to 5 moles propylene oxide to linear fatty alcohols with 8 to 22 carbon atoms, to fatty acids with 12 to 22 carbon atoms, to alkyl phenols with 8 to 15 carbon atoms in the alkyl group as well as alkylamines with 8 to 22 carbon atoms in the alkyl group;
(2) C_{12/18} fatty acid mono- and diesters of addition products of 1 to 30 moles ethylene oxide on glycerine;
(3) Glycerine mono- and diesters and sorbitol mono- and diesters of saturated and unsaturated fatty acids with 6 to 22 carbon atoms and their ethylene oxide addition products;
(4) Alkyl- and/or alkenyl mono- and -oligoglycosides with 8 to 22 carbon atoms in the alk(en)yl group and their ethoxylated analogs;
(5) Addition products of 15 to 60 moles ethylene oxide on castor oil and/or hydrogenated castor oil;
(6) Polyol esters and especially polyglycerine esters;
(7) Addition products of 2 to 15 moles ethylene oxide on castor oil and/or hydrogenated castor oil;
(8) Partial esters based on linear, branched, unsaturated or saturated C_{6/22} fatty acids, ricinoleic acid as well as 12-hydroxystearic acid and glycerine, polyglycerine, pentaerythritol, dipentaerythritol, sugar alcohols (e.g. sorbitol), alkyl glucosides (e.g. methyl glucoside, butyl glucoside, lauryl glucoside) as well as polyglucosides (e.g. cellulose);
(9) Mono, di- and trialkyl phosphates as well as mono-, di- and/or tri-PEG alkyl phosphates and salts thereof;
(10) Wool wax alcohols;
(11) Polysiloxane-polyalkyl-polyether copolymers or corresponding derivatives;
(12) Mixed esters of pentaerythritol, fatty acids, citric acid and fatty alcohol according to the Patent DE 1165574 and/or mixed esters of fatty acids with 6 to 22 carbon atoms, methyl glucose and polyols, preferably glycerine or polyglycerine,
(13) Polyalkylene glycols and
(14) Glycerine carbonate.

The addition products of ethylene oxide and/or propylene oxide on fatty alcohols, fatty acids, alkyl phenols, glycerine mono- and diesters as well as sorbitol mono- and diesters of fatty acids or on castor oil, represent known, commercially available products. They can be considered as mixtures of homologs, whose mean degree of alkoxylation corresponds to the ratio of amounts of ethylene oxide and/or propylene oxide, used for the addition reaction, and that of the substrate. C_{12/18} fatty acid mono- and diesters of addition products of ethylene oxide on glycerine are known from DE 2024051 as greasing agents for cosmetic preparations.

Alkyl and/or alkenyl mono- and oligoglycosides, their manufacture and use are known from the prior art. Their manufacture results particularly from the reaction of glucose or oligosaccharides with primary alcohols containing 8 to 18 carbon atoms. As far as the glycoside groups are concerned, both monoglycosides, in which a cyclic sugar group is glycosidically linked to the fatty alcohol, and also oligomeric glycosides, with a degree of oligomerization of preferably about 8, are suitable. In this context, the oligomerization degree is a statistical mean value based on the typical homolog distribution of such industrial products.

Typical examples of suitable polyglycerine esters are polyglyceryl-2-dipolyhydroxystearate (Dehymuls® PGPH), polyglycerine-3-diisostearate (Lameform® TGI), polyglyceryl-4-isostearate (Isolan® GI 34), polyglyceryl-3-oleate, diisostearoyl polyglyceryl-3-diisostearate (Isolan® PDI), polyglyceryl-3-methylglucose distearate (Tego Care® 450), polyglyceryl-3-beeswax (Cera Bellina®), polyglyceryl-4-caprate (polyglycerol caprate T2010/90), polyglyceryl-3-cetyl ether (Chimexane® NL), polyglyceryl-3-distearate (Cremophor® GS 32) and polyglyceryl polyricinoleate (Admul® WOL 1403) polyglyceryl dimerate isostearate and mixtures thereof.

Moreover, zwitterionic surfactants can be used as emulsifiers. Zwitterionic surfactants are designated as those surface-active compounds that carry at least a quaternary ammonium group and at least a carboxylate and a sulfonate group in the molecule. Particularly suitable zwitterionic surfactants are the so-called betaines such as the N-alkyl-N,N-dimethylammonium glycinates, for example the cocoalkyldimethylammonium glycinate, N-acylaminopropyl-N,N-dimethylammonium glycinates, for example the cocoacylaminopropyldimethylammonium glycinate, and 2-alkyl-3-carboxymethyl-3-hydroxyethyl imidazolines with 8 to 18 carbon atoms in each of the alkyl or acyl groups, as well as cocoacylaminoethylhydroxyethylcarboxymethyl glycinate. The fatty acid derivative known under the CTFA-description Cocamidopropyl Betaine is particularly preferred. Similarly, ampholytic surfactants are suitable emulsifiers. The ampholytic surfactants are understood to include such surface-active compounds that apart from a C₈₋₁₈ alkyl or acyl group, comprise at least one free amino group and at least one —COOH or —SO₃H group in the molecule, and are able to form internal salts. Examples of suitable ampholytic surfactants are N-alkylglycines, N-alkylamino propionic acids, N-alkylamino butyric acids, N-alkylimino dipropionic acids, N-hydroxyethyl-N-alkylamidopropylglycines, N-alkyltaurines, N-alkylsarcosines, 2-alkylamino propionic acids and alkylamino acetic acids, each with about 8 to 18 carbon atoms in the alkyl group. Particularly preferred ampholytic surfactants are the N-cocoalkylamino propionate, the cocoacylaminoethylamino propionate and besides the ampholytics, the quaternary emulsifiers can also be considered, wherein the esterquats, preferably methyl quaternized difatty acid triethanolamine ester salts are particularly preferred.

As greasing agents, substances such as lanolin and lecithin, as well as polyethoxylated or acylated lanolin and lecithin derivatives, polyol fatty acid esters, monoglycerides and fatty acid alkanolamides can be used, the last ones serving as foam stabilizers at the same time.

Pearlescent waxes include: alkylene glycol esters, especially ethylene glycol distearate; fatty acid alkanolamides, especially cocofatty acid diethanolamide; partial glycerides, especially monoglyceride of stearic acid; esters of polyfunctional, optionally hydroxy-substituted carboxylic acids with fatty alcohols with 6 to 22 carbon atoms, especially long chain esters of tartaric acid; fats, such as, for example fatty alcohols, fatty ketones, fatty aldehydes, fatty ethers and fatty carbonates, which have a total of at least 24 carbon atoms, especially lauron and distearyl ether; fatty acids like stearic acid, hydroxystearic acid or behenic acid, ring opened products of olefin epoxides having 12 to 22 carbon atoms with fatty alcohols with 12 to 22 carbon atoms and/or polyols having 2 to 15 carbon atoms and 2 to 10 hydroxyl groups and mixtures thereof.

Consistence agents primarily include fatty alcohols or hydroxyfatty alcohols having 12 to 22 and preferably 16 to 18 carbon atoms, besides partial glycerides, fatty acids or hydroxyfatty acids. A combination of these materials with alkyl oligo glucosides and/or fatty acid N-methylglucamides of the same chain length and/or polyglycerine poly-12-hydroxystearates is preferred.

Suitable thickeners are for example aerosil types (hydrophilic silicic acids), polysaccharides, especially xanthane gum, guar-guar, agar-agar, alginates and tyloses, carboxymethyl cellulose and hydroxyethyl cellulose, in addition, higher molecular weight polyethylene glycol mono- and -diesters of fatty acids, polyacrylates, (e.g. Carbopole® from Goodrich or Synthalene® from Sigma), polyacrylamides, polyvinyl alcohol and polyvinyl pyrrolidone, surfactants such as for example ethoxylated fatty acid glycerides, esters of fatty acids with polyols such as pentaerythritol or trimethylolpropane, fatty alcohol ethoxylates with narrowed homolog distribution or alkyl oligo glucosides as well as electrolytes like cooking salt and ammonium chloride.

Exemplary suitable cationic polymers are cationic cellulose derivatives, such as e.g. a quaternized hydroxyethyl cellulose, available under the trade name Polymer JR 400® from Amerchol, cationic starches, copolymers of diallylammonium salts and acrylamides, quaternized vinyl pyrrolidone/vinyl imidazole polymers, such as e.g. Luviquat® (BASF), condensation products of polyglycols with amines, quaternized collagen polypeptides, such as for example, lauryldimonium hydroxypropyl hydrolyzed collagen (Lamequat®L/Grünau), quaternized wheat polypeptides, polyethylene imine, cationic silicone polymers, such as for example amidomethicone, copolymers of adipic acid and dimethylaminohydroxypropyldiethylene triamine (Cartaretine®/Sandoz), copolymers of acrylic acid and dimethyldiallylammonium chloride (Merquat® 550/Chemviron), polyaminopolyamides, such as e.g. described in FR 2252840 A as well as their crosslinked water-soluble polymers, cationic chitin derivatives such as e.g. quaternized chitosan, optionally microcrystallinically dispersed, condensation products of dihaloalkylenes, such as e.g. dibromobutane with bisdialkylamines, such as e.g. bis-dimethylamino-1,3-propane, cationic guar-gum, such as e.g. Jaguar® CBS, Jaguar® C-17, Jaguar® C-16 from the Celanese Company, quaternized ammonium salt polymers, such as e.g. Mirapol® A-15, Mirapol® AD-1, Mirapol® AZ-1 from the Miranol Company.

Anionic, zwitterionic, amphoteric and non-ionic polymers include, for example, vinyl acetate-crotonic acid copolymers, vinyl pyrrolidone-vinyl acrylate copolymers, vinyl acetate-butyl maleate-isobornyl acrylate copolymers, methyl vinyl ether-maleic anhydride copolymers and their esters, uncrosslinked polyacrylic acids and those crosslinked with polyols, acrylamidopropyltrimethylammonium chloride-acrylate copolymers, octylacylamide-methyl methacrylate-tert.-butylaminoethyl methacrylate-2-hydroxypropyl methacrylate copolymers, polyvinyl pyrrolidone, vinyl pyrrolidone-vinyl acetate copolymers, vinyl pyrrolidone-dimethylaminoethyl methacrylate-vinyl caprolactam terpolymers as well as optionally derivatized cellulose ethers and silicones.

Exemplary suitable silicone compounds are dimethylpolysiloxanes, methylphenylpolysiloxanes, cyclic siloxanes as well as amino-, fatty acid-, alcohol-, polyether-, epoxy-, fluorine-, glycoside- and/or alkyl modified silicone compounds, which may be both liquid or also resinous at room temperature. Simethicones, which are mixtures of dimethicones having an average chain length of 200 to 300 dimethylsiloxane units and hydrated silicates, are also suitable. A detailed review of suitable volatile silicones is found in Todd et al., Cosm. Toil. 91, 27, (1976).

Typical examples of fats are glycerides; waxes include inter alia natural waxes such as e.g. candelilla wax, carnauba wax, Japan wax, esparto grass wax, cork wax, guarum wax, rice oilseed wax, raw sugar wax, ouricury wax, montan wax, beeswax, shellac wax, spermaceti, lanolin (wool wax), fowl fat, ceresine, ozokerite (mineral wax), petrolatum, paraffin waxes, microwaxes; chemically modified waxes (hard waxes), such as e.g. montan ester waxes, Sasol waxes, hydrogenated jojoba waxes as well as synthetic waxes, such as e.g. polyalkylene waxes and polyethylene glycol waxes.

Metal salts of fatty acids, such as e.g. magnesium-, aluminum- and/or zinc stearate or ricinoleate can be used as stabilizers.

Biogenetic active agents are understood to mean for example, tocopherol, tocopherol acetate, tocopherol palmitate, ascorbic acid, desoxyribonucleic acid, retinol, bisabolol, allantoin, phytanetriol, panthenol, AHA-acids, amino acids, ceramides, pseudoceramides, essential oils, plant extracts and vitamin complexes.

Cosmetic deodorants act against body odors, masking or eliminating them. Body odors result from the action of skin bacteria on apocrine sweat, whereby unpleasant smelling degradation products are formed. Accordingly, deodorants contain active principles, which act as germicides, enzyme inhibitors, odor absorbers or odor masks.

As germicides, which can be optionally added to the cosmetics according to the invention, basically all substances that are active against gram-positive bacteria are suitable, such as e.g. 4-hydroxybenzoic acid and its salts and esters, N-(4-chlorophenyl)-N′-(3,4-dichlorophenyl) urea, 2,4,4′-trichloro-2′-hydroxydiphenyl ether (Triclosan), 4-chloro-3,5-dimethylphenol, 2,2′-methylene-bis(6-bromo-4-chlorophenol), 3-methyl-4-(1-methylethyl) phenol, 2-benzyl-4-chlorophenol, 3-(4-chlorophenoxy)-1,2-propanediol, 3-iodo-2-propynylbutyl carbamate, chlorhexidine, 3,4,4′-trichlorocarbanilide (TTC), antibacterial fragrances, menthol, mint oil, phenoxyethanol, glycerine monolaurate (GML), diglycerine monocaprinate (DMC), salicylic acid-N-alkylamides such as, e.g. salicylic acid n-octylamide or salicylic acid n-decylamide.

Enzyme inhibitors can also be added to the inventive cosmetics. Examples of possible suitable enzyme inhibitors are esterase inhibitors. In this respect, trialkyl citrates are preferred, such as trimethyl citrate, tripropyl citrate, triisopropyl citrate, tributyl citrate and particularly triethyl citrate (Hydagen® CAT, Henkel KgaA, Düsseldorf/Germany). The substances inhibit the enzymatic activity and thereby reduce the odor formation. Additional substances that can be considered as esterase inhibitors are sterol sulfates or -phosphates, such as e.g. lanosterin-, cholesterin-, campesterin-, stigmasterin- and sitosterin sulfate or -phosphate, dicarboxylic acids and their esters, such as e.g. glutaric acid, monoethyl glutarate, diethyl glutarate, adipic acid, monoethyl adipate, diethyl adipate, malonic acid and diethyl malonate, hydroxycarboxylic acids and their esters such as e.g. citric acid, malic acid, tartaric acid or diethyl tartrate, as well as zinc glycinate.

Suitable odor absorbers are substances, which take up the odor-forming compounds and firmly block them. They reduce the partial pressures of the individual components and thus also reduce their rate of propagation. It is important that the perfumes remain unaffected by this. Odor absorbers have no activity against bacteria. They comprise as the major component, for example, a complex zinc salt of ricinoleic acid or specific, largely odor-neutral fragrances, which are known to the person skilled in the art as fixing agents, such as e.g. extracts of labdanum or styrax or specific abietic acid derivatives. Odoriferous substances or perfume oils act as masking agents and in addition to their function as masking agents, lend the deodorants their particular fragrance note. Exemplary perfume oils include mixtures of natural and synthetic odoriferous substances. Natural odoriferous substances are extracts of flowers, stalks and leaves, fruits, fruit skins, roots, branches, herbs and grasses, needles and twigs as well as resins and balsams. In addition, raw materials of animal origin such as e.g. civet and castoreum can be considered. Typical synthetic odoriferous compounds are products of the type of the esters, ethers, aldehydes, ketones, alcohols and hydrocarbons.

Antiperspirants reduce sweat formation by influencing the activity of the ecrinal sweat glands and thereby act against armpit moisture and body odor. Aqueous or anhydrous formulations of antiperspirants typically contain the following ingredients:
(a) astringent principles,
(b) oil components,
(c) non-ionic emulsifiers,
(d) coemulsifiers,
(e) consistency providers,
(f) auxiliaries such as e.g. thickeners or complexing agents and/or
(g) non-aqueous solvents such as e.g. ethanol, propylene glycol and/or glycerine.

Salts of aluminum, zirconium or zinc are the main suitable astringent antiperspirant active principles. Such suitable antihydrotically active substances are e.g. aluminum chloride, hydrated aluminum chloride, hydrated aluminum dichloride, hydrated aluminum sesquichloride and their complexes e.g. with 1,2-propylene glycol, aluminum hydroxy allantoinate, aluminum chloride tartrate, aluminum-zirconium trichlorohydrate, aluminum-zirconium tetrachlorohydrate, aluminum-zirconium pentachlorohydrate and their complex compounds e.g. with amino acids such as glycine.

The antiperspirants can also comprise standard oil-soluble and water-soluble auxiliaries in minor amounts. Such oil-soluble auxiliaries can be for example:
anti-inflammatory, skin-protecting or fragrant ethereal oils,
synthetic skin-protecting active principles and/or
oil-soluble perfume oils.

Typical water-soluble additives are e.g. preservatives, water-soluble fragrances, pH adjustors, e.g. buffer mixtures, water-soluble thickeners, e.g. water-soluble natural or synthetic polymers such as e.g. xanthane gum, hydroxyethyl cellulose, polyvinyl pyrrolidone or high-molecular weight polyethylene oxides.

Climbazole, Octopirox and zinc pyrethion can be used as anti-dandruff agents.

Usable film builders are for example, chitosan, microcrystalline chitosan, quaternized chitosan, polyvinyl pyrrolidone, vinyl pyrrolidone-vinyl acetate copolymers, polymers of the acrylic acid series, quaternized cellulose derivatives, collagen, hyaluronic acid or its salts and similar compounds.

As swelling agents for the aqueous phase, montmorillonite, mineral clays, Pemulen® as well as alkyl-modified Carbopol types (Goodrich) can be used. Further suitable polymers or swelling agents can be found in the review by R. Lochhead in Cosm. Toil. 108, 95 (1993).

The UV-light protective factors are understood for example to be organic substances (protective light filters) that are liquid or solid at room temperature and which are able to absorb UV-radiation and emit the resulting energy in the form of longer wavelength radiation, for example as heat. UVB filters can be oil-soluble or water-soluble. As oil-soluble substances, the following may be cited:
3-Benzylidenecamphor or 3-benzylidenenorcamphor and its derivatives, e.g. 3-(4-methylbenzylidene)camphor as described in EP 0693471 B1;
4-Aminobenzoic acid derivatives, preferably 4-dimethylamino)benzoic acid 2-ethylhexyl ester, 4-(dimethylamino)benzoic acid 2-octyl ester and 4-(dimethylamino)benzoic acid amyl ester;
Esters of cinnamic acid, preferably 2-ethylhexyl 4-methoxycinnamate, propyl 4-methoxycinnamate, isoamyl 4-methoxycinnamate, 2-ethylhexyl 2-cyano-3,3-phenylcinnamate (Octocrylene);
Esters of salicylic acid, preferably 2-ethylhexyl salicylate, 4-isopropylbenzyl salicylate, homomethyl salicylate;
Derivatives of benzophenone, preferably 2-hydroxy-4-methoxybenzophenone, 2-hydroxy-4-methoxy-4′-methylbenzophenone, 2,2′-dihydroxy-4-methoxybenzophenone;
Esters of benzalmalonic acid, preferably di-2-ethylhexyl 4-methoxybenzmalonate;
Triazine derivatives, such as e.g. 2,4,6-trianilino-(p-carbo-2′-ethyl-1′-hexyloxy)-1,3,5-triazine and octyl triazone, as described in EP 0818450 A1 or dioctyl butamido triazone (Uvasorb® HEB);
Propane-1,3-dione, such as e.g. 1-(4-tert.butylphenyl)-3-(4′-methoxyphenyl)propane-1,3-dione;
Ketotricyclo(5.2.1.0)decane derivatives, as described in EP 0 694 521 B1.

Water-soluble substances include:
2-Phenylbenzimidazole-5-sulfonic acid and its alkali metal, alkaline earth metal, ammonium, alkylammonium, alkanolammonium and glucammonium salts;
Sulfonic acid derivatives of benzophenones, preferably 2-hydroxy-4-methoxybenzophenone-5-sulfonic acid and its salts;
Sulfonic acid derivatives of 3-benzylidenecamphor, such as e.g. 4-(2-oxo-3-bornylidenemethyl)benzene sulfonic acid and 2-methyl-5-(2-oxo-3-bornylidene)sulfonic acid and its salts.

Typical UV-A filters particularly include derivatives of benzoylmethane, such as, for example 1-(4′-tert.-butylphenyl)-3-(4′-methoxyphenyl)propane-1,3-dione, 4-tert.-butyl-4′-methoxydibenzoylmethane (Parsol 1789), 1-phenyl-3-(4′-isopropylphenyl)-propane-1,3-dione as well as enamine compounds, as described in DE 19712033 A1 (BASF). Naturally, the UV-A and UV-B filters can also be added as mixtures. Besides the cited soluble materials, insoluble, light protective pigments, namely finely dispersed, metal oxides or salts can also be considered for this task. Exemplary suitable metal oxides are particularly zinc oxide and titanium oxide and also oxides of iron, zirconium, silicon, manganese, aluminum and cerium as well as their mixtures. Silicates (talc), barium sulfate or zinc stearate can be added as salts. The oxides and salts are already used in the form of pigments for skin care and skin protecting emulsions and decorative cosmetics. Here, the particles should have a mean diameter of less than 100 nm, preferably between 5 and 50 nm and especially between 15 and 30 nm. They can be spherical, however elliptical or other non-spherical shaped particles can also be used. The pigments can also be surface treated, i.e. hydrophilized or hydrophobized. Typical examples are coated titanium dioxides, such as, for example Titandioxid T 805 (Degussa) or Eusolex® T2000 (Merck). Hydrophobic coating agents preferably include silicones and among them specifically trialkoxy octylsilanes or Simethicones. Sun-protection agents preferably contain micropigments or nano-pigments. Preferably, micronized zinc oxide is used. Further suitable UV light protection filters may be found in the review by P. Finkel in SöFW-Journal, Volume 122 (1996), p. 543.

As well as both above-cited groups of primary light protective materials, secondary light protective agents of the antioxidant type can also be used, which interrupt photochemical chain reactions that are propagated when the UV-radiation penetrates the skin. Typical examples are amino acids (e.g. glycine, histidine, tyrosine, tryptophan) and their derivatives, imidazoles (e.g. urocanic acid) and their derivatives, peptides such as D,L-carnosine, D-carnosine, L-carnosine and their derivatives (e.g. anserine), carotinoides, carotenes (e.g. α-carotene, β-carotene, lycopine) and their derivatives, chlorogenic acids and their derivatives, liponic acids and their derivatives (e.g. dihydroliponic acid), aurothioglucose, propylthiouracil and other thioles (e.g. thioredoxine, glutathione, cystein, cystine, cystamine and their glycosyl-, N-acetyl-, methyl-, ethyl-, propyl-, amyl-, butyl- and lauryl-, palmitoyl-, oleyl-, γ-linoleyl-, cholesteryl- and glyceryl esters) as well as their salts, dilauryl thiodipropionate, distearyl thiodipropionate, thiodipropionic acid and their derivatives (esters, ethers, peptides, lipids, nucleotides, nucleosides and salts) as well as sulfoximine compounds (e.g. buthionine sulfoximines, homocystein sulfoximine, butionine sulfone, penta-, hexa-, heptathionine sulfoximine) in very minor compatible doses (e.g. pmol to μmol/kg), further (metal) chelates (e.g. α-hydroxyfatty acids, palmitic acid, phytinic acid, lactoferrin), α-hydroxyacids (e.g. citric acid, lactic acid, malic acid), humic acid, gallic acid, gall extracts, bilirubin, biliverdin, EDTA, EGTA and their derivatives, unsaturated fatty acids and their derivatives (e.g. γ-linolenic acid, linoleic acid, oleic acid), folic acid and their derivatives, ubiquinone and ubiquinol and their derivatives, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate) as well as coniferyl benzoate of benzoic resin, rutinic acid and their derivatives, α-glycosyl rutine, ferula acid, furfurylideneglucitol, carnosine, butylhydroxytoluene, butylhydroxyanisol, nordihydroguajac resin acid, nordihydroguajaret acid, trihydroxybutyrophenone, uric acid and their derivatives, mannoses and their derivatives, superoxide-dismutase, zinc and its derivatives (e.g. ZnO, ZnSO₄) selenium and its derivatives (e.g. selenium-methionine), stilbenes and their derivatives (e.g. stilbene oxide, trans stilbene oxide) and the inventively suitable derivatives (salts, esters, ethers, sugars, nucleotides, nucleosides, peptides and lipids) of these cited active substances.

To improve the flow properties, hydrotropes can also be added, such as, for example, ethanol, isopropyl alcohol, or polyols. Polyols, which are considered, possess preferably 2 to 15 carbon atoms and at least two hydroxyl groups. The polyols can comprise further functional groups, especially amino groups, or can be modified by nitrogen. Typical examples are
Glycerine;
Alkylene glycols, such as, for example, ethylene glycol, diethylene glycol, propylene glycol, butylene glycol, hexylene glycol as well as polyethylene glycols with an average molecular weight of 100 to 1000 daltons;
Industrial oligoglycerine mixtures with a degree of self-condensation of 1.5 to 10, such as for instance industrial diglycerine mixtures with a diglycerine content of 40 to 50 wt. %;
Methylol compounds such as in particular trimethylol ethane, trimethylol propane, trimethylol butane, pentaerythritol and dipentaerythritol;
Lower alkyl glucosides, particularly those with 1 to 8 carbon atoms in the alkyl group, such as, for example methyl and butyl glucoside;
Sugar alcohols with 5 to 12 carbon atoms, such as, for example sorbitol or mannitol,
Sugars with 5 to 12 carbon atoms, such as, for example glucose or saccharose;
Amino sugars, such as for example glucamine;
Dialcoholamines, such as diethanolamine or 2-amino-1,3-propanediol.

Suitable preservatives are, for example phenoxyethanol, formaldehyde solution, parabene, pentanediol or sorbic acid as well as the further classes of substances described in Appendix 6, part A and B of the Cosmetic Regulation. Insect repellents include N,N-diethyl-m-toluamide, 1,2-pentanediol, or ethyl butylacetylamino propionate; suitable self-tanning agents include dihydroxyacetone.

As perfume oils, the known mixtures of natural and synthetic odoriferous substances can be cited. Natural aromas are extracts of flowers (lilies, lavender, roses, jasmine, neroli, ylang ylang), stalks and leaves (geranium, patchouli, petit grain), fruits (aniseed, coriander, caraway, juniper), fruit skins (bergamot, lemons, oranges), roots (mace, angelica, celery, cardamom, costic, iris, calmus), wood (pine, sandal, guava, cedar, rose wood), herbs and grasses (tarragon, lemon grass, sage, thyme), needles and twigs (spruce, fir, scotch pine, larch), resins and balsam (galbanum, elemi, benzoin, myrrh, olibanum, opoponax). In addition, raw materials of animal origin such as e.g. civet and castoreum can be considered. Typical synthetic odoriferous compounds are products of the type of the esters, ethers, aldehydes, ketones, alcohols and hydrocarbons.

As colorants, those substances suitable and approved for cosmetic purposes can be used, as summarized, for example in the publication “Kosmetische Farbemittel” of the Colorant Commission of the Deutsche Forschungsgemeinschaft, Verlag Chemie, Weinheim, 1984, pp. 81-106. These colorants are typically used in concentrations of 0.001 to 0.1 wt. %, based on the total mixture.

The total content of auxiliaries and additives can be 1 to 50, preferably 5 to 40 wt. %, based on the composition. The composition can be manufactured using customary cold or hot processes; preferably according to the phase inversion temperature method.

A further subject matter of the invention is an oxidative dyestuff for dyeing keratin fibers, comprising inventively useable amadoriases and especially amadoriases according to the invention. Keratin fibers are understood to mean wool, feathers, skins and particularly human hair.

To manufacture the inventive oxidizing agent, the oxidative dyestuff precursors as well as the amadoriases are incorporated into a suitable aqueous carrier in the absence of atmospheric oxygen. Such carriers are, for example, thickened aqueous solutions, creams (emulsions), gels or surfactant-containing preparations, for example, shampoos or foam aerosols or other preparations that are suitable for use on the hair.

Anhydrous powders are fundamentally suitable carriers; in this case the oxidative dyestuff is dissolved or dispersed in water immediately before use. Preferred carrier components are
wetting agents and emulsifiers
thickeners
reducing agents (antioxidants)
hair care additives
fragrances and
solvents such as e.g. water, glycols or lower alcohols.

Exemplary suitable wetting agents and emulsifiers are anionic, zwitterionic, ampholytic and non-ionic surfactants. Cationic surfactants can also be employed to achieve certain effects.

Suitable thickeners are the water-soluble high molecular weight polysaccharide derivatives or polypeptides, e.g. cellulose ethers or starch ethers, gelatines, plant gums, biopolymers (Xanthane gum) or water-soluble synthetic polymers such as e.g. polyvinyl pyrrolidone, polyvinyl alcohol, polyethylene oxides, polyacrylamides, polyurethanes, polyacrylates and others.

Furthermore, surfactant-containing preparations can also be thickened by solubilization or emulsification of polar lipids. Such lipids are e.g. fatty alcohols having 12-18 carbon atoms, (free) fatty acids having 12-18 carbon atoms, partial glycerides of fatty acids, sorbitol esters of fatty acids, fatty acid alkanolamides, low ethoxylated fatty acids or fatty alcohols, lecithin, sterin. Finally, gelled carriers can also be produced based on aqueous soap gels, e.g. of ammonium oleate.

Reducing agents (antioxidants) that are added to the carrier in order to prevent a premature oxidative development of the dyestuff prior to the application on the hair are e.g. sodium sulfite or sodium ascorbate.

Hair care additives can be e.g. fats, oils or waxes in emulsified form, structure-promoting additives such as e.g. glucose or pyridoxine, freshening components such as e.g. water-soluble proteins, protein decomposition products, amino acids, water-soluble cationic polymers, silicones, vitamins, panthenol or vegetal extracts.

Finally, fragrances and solvents such as e.g. glycols like 1,2-propylene glycols, glycerine, glycol ethers like e.g. butyl glycol, ethyl glycol or lower monohydric alcohols like ethanol or isopropanol can be comprised.

In addition, still more auxiliaries can be comprised, which improve the stability and application characteristics of the oxidative dye, e.g. complexants like EDTA, NTA or organo phosphonates, swelling and penetration agents such as e.g. urea, guanidine, hydrogen carbonates, buffer salts such as e.g. ammonium chloride ammonium citrate, ammonium sulfate or alkanolammonium salts and optionally propellant gases.

A further subject matter of the invention is a composition for oral, dental or dental prostheses care, especially prostheses cleaners, comprising amadoriases according to the invention for bleaching or for disinfection.

For partial dentures or dentures, the presentation is suitable both as denture cleaning tablets and also as mouth rinses or mouth water, or as toothpaste.

The inventive oral, dental and/or dental prostheses care compositions can exist, for example, as mouth water, gels, liquid toothpaste, viscous toothpaste, denture cleaners or adhesive creams for prostheses.

For this, the inventively employable amadoriases have to be incorporated in a suitable carrier.

Suitable carriers can also be e.g. powdered preparations or aqueous-alcoholic solutions that comprise 0 to 15 wt. % ethanol, 1 to 1.5 wt. % aromatic oils and 0.01 to 0.5 wt. % sweeteners as the mouth wash, or 15 to 60 wt. % ethanol, 0.05 to 5 wt. % aromatic oils, 0.1 to 3 wt. % sweeteners and optional additional auxiliaries as the mouth wash concentrate that is diluted with water before use. Consequently, the concentration of the components must be chosen at a high enough level such that during the application after dilution they do not fall below the cited lower concentration limits.

However, gels and more or less flowable pastes can also serve as carriers and which can be pressed out of flexible plastic containers or tubes and applied to the teeth by means of a toothbrush. Such products comprise higher quantities of moisturizers and binders or consistency regulators and polishing components. Moreover, these preparations also comprise aromatic oils, sweeteners and water.

Moisturizers can comprise and use, for example, glycerine, sorbitol, xylitol, propylene glycols, polyethylene glycols or mixtures of these polyols, in particular those polyethylene glycols with molecular weights from 200 to 800 (from 400-2000). The preferred moisturizer is sorbitol, comprised in an amount of 25-40 wt. %.

Condensed phosphates in the form of their alkali metal salts, preferably in the form of their sodium or potassium salts, can be included as anti-tartar active substances and as demineralization inhibitors. Due to the hydrolytic effect, the aqueous solutions of these phosphates are alkaline. The pH of the inventive oral, dental and/or dental prostheses care compositions is adjusted to the preferred value of 7.5-9 by the addition of acid.

Mixtures of various condensed phosphates or also hydrated salts of condensed phosphates can also be added. The specified quantities of 2-12 wt. % refer, however, to the anhydrous salts. A sodium or potassium tripolyphosphate in a concentration of 5 to 10 wt. % is preferred as the condensed phosphate.

A preferred comprised active substance is a caries-inhibiting fluorine compound, preferably from the group of fluorides or monofluorophosphates, in an amount of 0.1 to 0.5 wt. % fluorine. Suitable fluorine compounds are e.g. sodium monofluorophosphate (Na₂PO₃F), potassium monofluorophosphate, sodium or potassium fluoride, tin fluoride or the fluoride of an organic amino compound.

Natural and synthetic water-soluble polymers, such as carrageen, traganth, guar, starch and their non-ionic derivatives such as e.g. hydroxypropyl guar, hydroxyethyl starch, cellulose ethers such as e.g. hydroxyethyl cellulose or methylhydroxypropyl cellulose serve as exemplary binding agents and consistence regulators. Also agar-agar, xanthane gum, pectins, water-soluble carboxyvinyl polymers (e.g. Carbopol types) polyvinyl alcohol, polyvinyl pyrrolidone, higher molecular weight polyethylene glycols (molecular weight 10³ to 10⁶ D). Additional substances that are suitable for controlling viscosity are layered silicates such as e.g. montmorillonite clays, colloidal thickening silicas, e.g. aerogel silica or pyrogenic silicas.

As the polishing components all the polishing agents known for this can be added, but preferably precipitated and gelled silicas, aluminum hydroxide, aluminum silicate, aluminum oxide, aluminum oxide trihydrate, insoluble sodium metaphosphate, calcium pyrophosphate, calcium hydrogen phosphate, dicalcium phosphate, chalk, hydroxy apatite, hydrotalcite, talcum, magnesium aluminum silicate (Veegum®), calcium sulfate, magnesium carbonate, magnesium oxide, sodium aluminum silicate, e.g. zeolite A or organic polymers e.g. polymethyl acrylate. The polishing agents are advantageously used in smaller amounts of e.g. 1 to 10 wt. %.

The organoleptic properties of the inventive dental and/or oral care products can be improved by the addition of aromatic oils and sweeteners. All the natural and synthetic aromas suited for oral-, dental- and/or dental prostheses can be considered as the aromatic oils. Natural aromas can be used both in the form of ethereal oils isolated from drugs and also from individual components isolated from them. Preferably at least one aromatic oil is comprised from the group peppermint oil, spearmint oil, anisole, caraway oil, eucalyptus oil, fennel oil, cinnamon oil, geranium oil, sage oil, oil of thyme, marjoram oil, oil of basil, lemon oil, gaultheria oil or one or a plurality of the synthetically produced isolated components of these oils. The most important components of the cited oils are e.g. menthol, carvone, anethol, cineol, eugenol, cinnamaldehyde, geraniol, citronellol, linalool, salven, thymol, terpenes, terpineol, methyl chavicol and methyl salicylate. Further suitable aromas are e.g. menthyl acetate, vanillin, ionone, linalyl acetate, rhodinol and piperitone. Either natural sugars, such as sucrose, maltose, lactose and fructose, or synthetic sweeteners such as e.g. the sodium salt of saccharin, sodium cyclamate or aspartame are suitable edulcorants.

In particular, alkyl and/or alkenyl (oligo) glycosides can be used as the surfactants. Their manufacture and use as surface active materials are known, for example, from U.S. Pat. No. 3,839,318, U.S. Pat. No. 3,707,535, U.S. Pat. No. 3,547,828, DE-A-19 43 689, DE-A-20 36 472 and DE-A-30 01 064 as well as EP-A-77 167. As far as the glycoside groups are concerned, both monoglycosides (x=1), in which a pentose or hexose group is glycosidically linked to a primary alcohol having 4 to 16 carbon atoms, and also oligomeric glycosides, with a degree of oligomerization x up to 10, are suitable. In this context, the oligomerization degree is a statistical mean value based on the typical homolog distribution of such industrial products.

Preferred suitable alkyl and/or alkenyl (oligo) glycosides are an alkyl and/or alkenyl (oligo) glucoside of the Formula RO(C₆H₁₀O)ₓ—H, in which R is an alkyl and/or an alkenyl group with 8 to 14 carbon atoms and x has a mean value of 1 to 4. Alkyl oligo glucosides based on hydrogenated C12/14 coco alcohol with a DP of 1 to 3 are particularly preferred. The alkyl and/or alkenyl glycoside surfactant can be used very sparingly, amounts of 0.005 to 1 wt. % being already sufficient.

Apart from the cited alkyl glucoside surfactants, other non-ionic, ampholytic and cationic surfactants can also be comprised, examples being:

Fatty alcohol polyglycol ether sulfonates, monoglyceride sulfates, monoglyceride ether sulfates, mono and/or dialkyl sulfosuccinates, fatty acid isethionates, fatty acid sarcosinates, fatty acid taurides, fatty acid glutamates, ether carboxylic acids, fatty acid glucamides, alkylamido betaines and/or protein-fatty acid condensates, the last preferably on the basis of wheat proteins. A non-ionic solubilizer from the group of the surface-active compounds can be required, particularly for solubilizing the mostly water-insoluble aromatic oils. For example, ethoxylated fatty acid glycerides, ethoxylated fatty acid sorbitol partial esters or fatty acid partial esters of glycerine ethoxylates or sorbitol ethoxylates are suitable for this task. Solubilizers from the group of the ethoxylated fatty acid glycerides primarily include addition products of 20 to 60 moles ethylene oxide on mono and diglycerides of linear fatty acids having 12 to 18 carbon atoms or on triglycerides of hydroxyfatty acids such as oxystearic acid or ricinoleic acid. Further suitable solubilizers are ethoxylated fatty acid sorbitol partial esters; they are preferably addition products of 20 to 60 moles ethylene oxide on monoesters of sorbitol and diesters of sorbitol with fatty acids having 12 to 18 carbon atoms. Equally suitable solubilizers are fatty acid partial esters of glycerine ethoxylates or sorbitol ethoxylates; they are preferably monoesters and diesters of C₁₂-C₁₈ fatty acids and addition products of 20 to 60 moles ethylene oxide on 1 mole glycerine or on 1 mole sorbitol.

The inventive oral, dental and/or dental prostheses care compositions preferably comprise addition products of 20 to 60 moles ethylene oxide on hydrogenated or non-hydrogenated castor oil (i.e. on oxystearic acid triglyceride or ricinoleic acid triglyceride), on glycerine mono and/or distearate or on sorbitol mono and/or distearate, as the solubilizer for the optionally comprised aromatic oils.

Additional typical additives for the oral, dental and/or dental prostheses care agents are e.g.
pigments, e.g. titanium dioxide, and/or colorants,
pH adjustors and buffer substances like e.g. sodium bicarbonate, sodium citrate, sodium benzoate, citric acid, phosphoric acid or acidic salts e.g. NaH₂PO₄,
wound-healing and anti-inflammatory substances such as, for example, allantoin, urea, panthenol, azulene or chamomile extract,
additional active substances to combat tartar such as e.g. organo phosphonates, e.g. hydroxyethane diphosphonates or azacycloheptane diphosphonate,
preservatives such as e.g. sorbic acid salts, p-hydroxybenzoates,
plaque inhibitors such as e.g. hexachlorophene, Chlorhexidin, Hexetidin, Triclosan, bromochlorophene, phenyl salicylate.

In a particular embodiment, the composition is a mouthwash, a mouth water, a denture cleaner or a denture adhesive.

For inventively preferred denture cleaners, particularly denture cleaning tablets and powder, besides the ingredients already mentioned for oral, dental and/or dental prostheses care, peroxy compounds such as for example peroxyborate, peroxymonosulfate or percarbonate are also suitable. They have the advantage that besides the bleaching activity, they simultaneously act as deodorizers and/or as disinfectants. Such peroxy compounds are added in denture cleaners in the range between 0.01 and 10 wt. %, particularly between 0.5 and 5 wt. %.

Enzymes, such as proteases and carbohydrases are also suitable as additional ingredients for decomposing proteins and carbohydrates. The pH can be between pH 4 and pH 12, in particular between pH 5 and pH 11.

In addition, further auxiliaries are required for the denture cleaning tablets, such as for example agents that initiate an effervescence, such as e.g. CO₂-releasing materials such as sodium hydrogen carbonate, fillers, e.g. sodium sulfate or dextrose, lubricants, e.g. magnesium stearate, flow regulators, such as for example colloidal silicon dioxide and granulating agents, such as the already cited high molecular weight polyethylene glycols or polyvinyl pyrrolidone.

Denture adhesives can be offered as powders, creams, films or liquids and support the adhesion of the dentures.

Natural and synthetic swelling agents are suitable as active principles. Besides alginates, vegetal gums, such as e.g. gum arabicum, traganth and karayi gum as well as natural rubber are to be understood as natural swelling agents. In particular, alginates and synthetic swelling agents, such as e.g. sodium carboxymethyl cellulose, high molecular weight ethylene oxide copolymers, salts of polyvinyl ether-maleic acid and polyacrylamides are suitable.

In particular, hydrophobic foundations, especially hydrocarbons, such as for example white Vaseline (DAB) or paraffin oil, are especially suitable as the auxiliaries for pasty and liquid products.

The following examples illustrate the invention without, however, restricting it in any way:

### Example 1

### Investigation of Enzyme Concentrates for the Presence of an Amadoriase Activity with a Highly Denatured Stain as the Substrate

Sample: Enzyme concentrate of Bacillus licheniformis
Stains as the substrate:
1. from EMPA: blood/milk/ink on cotton: product no. 116 obtainable from the Company: Eidgenössische Material- and Prüfanstalt (EMPA) Testmaterialien AG, St. Gallen (Switzerland)
2. Complete egg/pigment on cotton: product no. 10N obtainable from the company wfk Testgewebe GmbH; Brüggen-Bracht, Germany.

Preparation of the stains:

The stains were cut up into 2 mm×4 mm pieces, for each preparation ca. 50 mg rags were employed.

The detection of the amadoriase activity was made using a H₂O₂ detection plate. The plate was prepared as follows:

1 g agar was boiled up in 50 mL 120 mM Tris-HCl buffer pH 8.5. In a second solution, 26.8 mg of 4-chloronaphthene and horse radish peroxidase (2 mL of a 54 U/mL solution) were dissolved in 50 mL bidistilled water. After the solutions were combined, the agar plates were poured. When cooled, small holes were punched (ca. 0.5 cm diameter), into which the enzyme sample was added. References: Simon Delagrave, Dennis I Murphy, Jennifer L. Rittenhouse Pruss, Anthony M. Maffia, III, Barry L. Marrs, Edward I Bylina, William I Coleman, Christina L. Grek, Michael R. Dilworth, Mary M. Yang & Douglas C. Youvan “Application of a very high-throughput digital imaging screen to evolve the enzyme galactose oxidase” Protein Engineering 2001 vol. 14, no. 4, pp 261-267 Bylina, E J., Grek, C. L., Coleman, W I & Youvan, D. C. “Directed evolution and solid phase enzyme Screening” Proc. SPIE 2000 Vol. 3926, pp. 186-191, Advances in Nucleic Acid and Protein Analyses, Manipulation, and Sequencing

Test procedure:

A 50 mg stain was placed into each of the 2.2 mL Eppendorf cups. 700 μL 120 mM Tris HCl buffer pH 8.5 and 30 μL of a 30 mM hydroxylamine sulfate solution as the Katalase inhibitor (1 mM) were added. 300 μL aliquots of the culture supernatant, 100 μL of the enzyme concentrates were added to each of the cups.

The incubation temperature was 37° C., the agar plates being incubated on a shaker (Intelli-Mixer RM-2L from LFT-Labortechnik, program F1, 40 rpm) for up to 22 hours. After 2.5 and 22 hours incubation, 30 μL samples were removed from each cup and added to a H₂O₂ detection plate (see above).). A blue coloration shows a positive H₂O₂ detection.

Results: The enzyme concentrate showed a definite blue coloration with both stains. This corresponds to a detection of amadoriase activity,

### Example 2

### Investigation of Enzyme Concentrates for the Presence of an Amadoriase Activity with Synthetic Amadori Raw Products as the Substrate

Substrate: Products comprising Amadori compounds: Description of the synthesis: 0.5 mol glucose, 0.1 mol glycine, 10 g sodium metabisulfite and 10 mL water was incubated in a water bath at 95° C. for 4 hours. Parallel to this, a negative control was prepared, which comprised the same substances, but was not heated. (Hironaga Hashiba “Participation of amadori rearrangement products and carbonyl Compounds in oxygen-dependent browning of soy sauce”, 3. Agric. Food Chem., 1976 VoI 24, pp. 70-74)

Sample:

The substrate (synthesis product+negative control) was tested with the enzyme concentrate in two concentrations.

Starting materials:
100 μL sample
100 and 500 μL substrate
30 μL 30 mM hydroxylamine sulfate
ad 1 mL 120 mM Tris-HCl pH 8.5

Incubation at 37° C., 40 rpm, for 1.15 h. Test of each 30 μL by means of H₂O₂ detection plates.

Results:

The enzyme concentrate showed a blue coloration with the substrate.

In this method, the minimum detectable H₂O₂ concentration is for an enzyme activity that produces 1.5 ng H₂O₂ in 30 minutes.

Assay of amadoriase activity

The amadoriase activity was measured at 37° C. by the formation of a quinone colorant A555 (E, 39.2 cm²/_mol) with a suitable spectrophotometer. The reaction mixture comprised 100 mM potassium phosphate (pH 8.0); 2.7 purpurogallin units of peroxidase; 0.45 mM 4-aminoantipyrine; 0.5 mM N-ethyl-N-(2-hydroxy-3-sulfopropyl)-m-toluidine, and 5.0 mM Amadori product in a total volume of 3 ml. Under these conditions the reaction ran linearly for a period of 1 to 3 minutes. An enzyme activity unit was defined as the quantity of enzyme that produced 0.5 μmol quinone colorant per minute. (Ryoichi Sakaue & Naoki Kajiyama “Thermostabilisation of bacterial fructosyl-amino-acid oxidase by directed evolution” Appl. Environ. Micro., Vol. 69, 2003, 139-145)

### Example 3

### Amadoriase

### Laundry Test

Enzymes:
Amadoriase obtained from Sigma-Aldrich Corporation, Box 14508, St. Louis Mo. 63178 USA
Protease concentrate from Bacillus licheniformis (examples are BLAP-S or BLAP-X from the Company HENKEL KGaA)
Catalase obtained from Merck KGaA, Darmstadt, Germany

Buffer:
100 mM Tris/HCl pH 8.5 (enzyme solution)

Stains:
Blood/milk/ink on cotton: product no. C5 from CFT B.V. Vlaardingen, Holland
Complete egg/pigment on cotton: product no. 10N obtainable from the company wfk Testgewebe GmbH; Brüggen-Bracht, Germany, cut up into small pieces.

Laundry detergent matrix:

TABLE 1
General formulation for a textile laundry detergent

| Chemical Name | [wt. %] pure substance |
|---|---|
| Xanthane gum | 0.3-0.5 |
| Antifoaming agent | 0.2-0.4 |
| Glycerine | 6-7 |
| Ethanol | 0.3-0.5 |
| FAEOS | 4-7 |
| Non-ionic surfactant (FAEO, APG, i.a.) | 24-28 |
| Boric acid | 1 |
| Sodium citrate × 2 H₂O | 1-2 |
| Caustic soda | 2-4 |
| Coconut fatty acid | 14-16 |
| HEDP | 0.5 |
| PVP | 0-0.4 |
| Optical brightener | 0-0.05 |
| Dye | 0-0.001 |
| Perfume | 0-2 |
| H₂O, demin. | remainder |

Test materials (1 ml) in 48 well plates:

| Volumes | | Solution |
|---|---|---|
| 420 | μl | 161-966 mg textile laundry detergent in |
| | | 42 ml water or buffer |
| 30-530 | μl | 10-100 U/ml protease concentrate of |
| | | Bacillus licheniformis (Examples are |
| | | BLAP-S or BLAP-X from Henkel KgaA) |
| 20-520 | μl | 100-500 U/ml catalase |
| 30-530 | μl | 10-100 U/ml amadoriase |

| remainder | H₂O |
|---|---|
| | Stain ø 1 cm |

Incubation: 60 Min., 40° C., ca. 600 rpm

After incubation: rinsing (3×) the stains, drying and fixing

Measurement of the intensity with a colorimeter Cm508d (Minolta)

For this test, round test fabric pieces (diameter 10 mm) were incubated in a 24-well microtiter plate in 1 ml of wash liquor for 30 min at 37° C. with agitation at 100 rpm. The wash liquor comprised protease (protease concentrate of Bacillus licheniformis, examples are BLAP-S or BLAP-X Darmstadt, Germany) and/or amadoriase (obtained from Sigma-Aldrich Corporation, Box 14508, St. Louis Mo. 63178 USA). Each test was carried out as a triple determination against a triply determined control with only catalase.

After washing, the degree of whiteness of the washed textiles was measured in comparison with a whiteness standard (d/8, Ø 8 mm, SCI/SCE), which had been set at 100% (L-value determination). The measurement was made using a colorimeter (Minolta Cm508d) with a light setting of 107D65. The results were expressed as percent power, wherein the difference of the reflectance value of the base laundry detergent without enzymes to that with protease and with catalase was set to 100%.

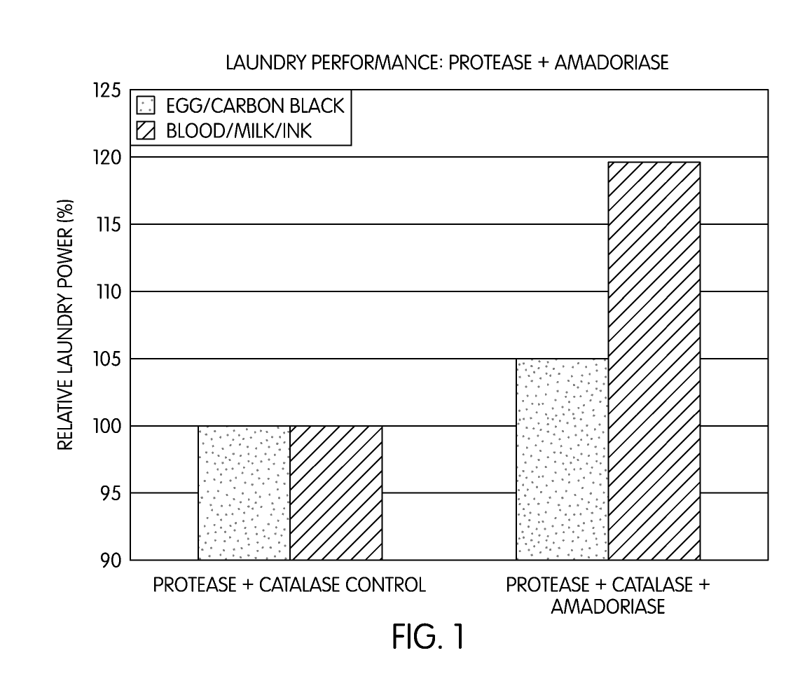

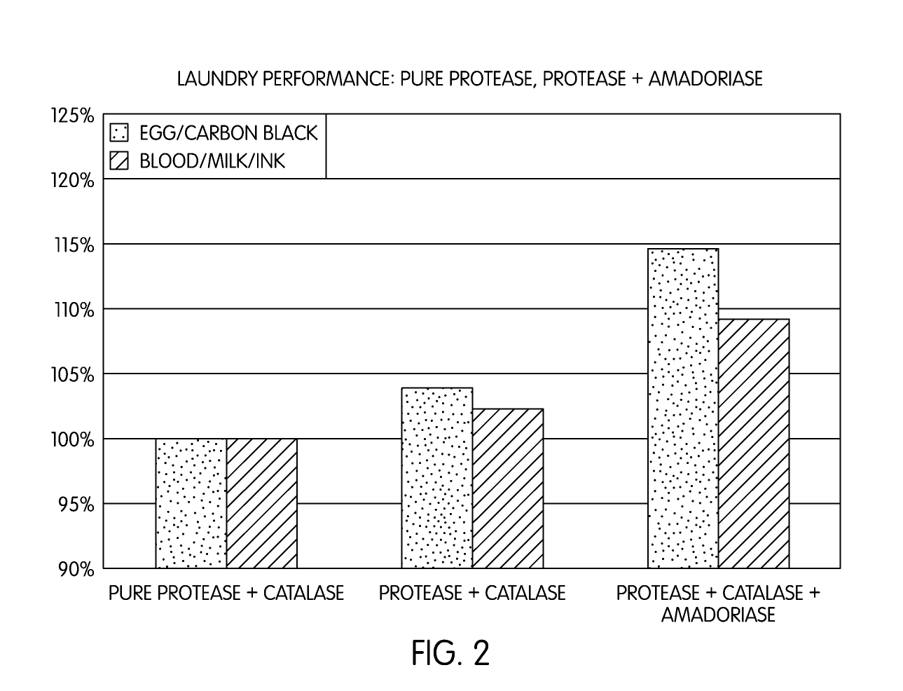

The results of the laundry test were presented in FIG. 1. FIG. 2 shows the tests of a laundry test that corresponds to that shown in FIG. 1, in which the protease concentrate was of Bacillus licheniformis, but was purified by the use of a conventional biochemical method.

The results show a significantly superior power of the inventive combination of amadoriase over that without amadoriase. The better result was obtained with the blood/milk/ink stain. This seems logical if one takes into account the composition and preparation of the blood/milk/ink stain: more Amadori product is expected in the blood/milk/ink stain than in the egg-carbon black stain, as significantly more sugar (for example glucose from blood and lactose from milk) can react with very much protein (membrane protein).

1. A washing or cleaning composition comprising at least one amadoriase, and optionally comprising a substrate for the amadoriase.

2. The composition of claim 1, further comprising at least one protease, amylase, cellulose, or perhydrolase.

3. The composition of claim 1, further comprising at least one bleach activator.

4. The composition of claim 1, further comprising at least one bleach catalyst.

5. The composition of claim 1, wherein the amadoriase is isolated from a fungal cell or a bacteria cell.

6. The composition of claim 5, wherein the fungal cell is of the genus Aspergillus.

7. The composition of claim 5, wherein the bacteria cell is of the genus Bacillus.

8. The composition of claim 5, wherein the amadoriase is isolated from a fungal cell or a bacteria cell selected from the group consisting of Agrobacterium tumefaciens, Arthrobacter sp., Aspergillus clavatus, Aspergillus fumigatus, Aspergillus nidulans, Aspergillus oryzae, Aspergillus terreus, Bacillus halodurans, Candida boidinii, Coniochaeta sp., Corynebacterium sp., Cryptococcus neoformans, Debaryomyces hansenii, Eupenicillium terrenum, Filobasidiella neoformans, Gibberella zeae, Neosartorya fischeri, Ulocladium sp., Fusarium oxysporum, Acremonium, Bacillus cereus, Bacillus clausii; Brevibacterium linens, Thermobifidafusca; Bacillus anthracis, Bacillus thuringiensis, Eobacillus kaustophilus, Bacillus subtilis, Oceanobacillus iheyensjs, Geobacillus kaustophilus, Penicillium spp., Penicillium janthinellum, Pichia sp., Bacillus licheniformis and Schizosaccharomyces pombe.

9. The composition of claim 1, wherein the amadoriase has an amino acid sequence having at least 95% identity with SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, or SEQ ID NO:26.

10. The composition of claim 1, wherein the amadoriase and the substrate for the amadoriase are encapsulated by an agent that is impermeable to the amadoriase and/or the substrate at room temperature or in the absence of water and that becomes permeable for the amadoriase and/or the substrate at a temperature higher than room temperature or in the presence of water.

11. A body care or cosmetic composition comprising at least one amadoriase.

12. The composition of claim 11, further comprising at least one protease, amylase, cellulose, or perhydrolase.

13. The composition of claim 11, further comprising at least one bleach activator.

14. The composition of claim 11, further comprising at least one bleach catalyst.

15. The composition of claim 11, wherein the amadoriase is isolated from a fungal cell or a bacteria cell.

16. The composition of claim 15, wherein the fungal cell is of the genus Aspergillus.

17. The composition of claim 15, wherein the bacteria cell is of the genus Bacillus.

18. The composition of claim 15, wherein the amadoriase is isolated from a fungal cell or a bacteria cell selected from the group consisting of Agrobacterium tumefaciens, Arthrobacter sp., Aspergillus clavatus, Aspergillus fumigatus, Aspergillus nidulans, Aspergillus oryzae, Aspergillus terreus, Bacillus halodurans, Candida boidinii, Coniochaeta sp., Corynebacterium sp., Cryptococcus neoformans, Debaryomyces hansenii, Eupenicillium terrenum, Filobasidiella neoformans, Gibberella zeae, Neosartorya fischeri, Ulocladium sp., Fusarium oxysporum, Acremonium, Bacillus cereus, Bacillus clausii; Brevibacterium linens, Thermobifidafusca; Bacillus anthracis, Bacillus thuringiensis, Eobacillus kaustophilus, Bacillus subtilis, Oceanobacillus iheyensjs, Geobacillus kaustophilus, Penicillium spp., Penicillium janthinellum, Pichia sp., Bacillus licheniformis and Schizosaccharomyces pombe.

19. The composition of claim 11, wherein the amadoriase has an amino acid sequence having at least 95% identity with SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, or SEQ ID NO:26.

20. The composition of claim 11, wherein the amadoriase and the substrate for the amadoriase are encapsulated by an agent that is impermeable to the amadoriase and/or the substrate at room temperature or in the absence of water and that becomes permeable for the amadoriase and/or the substrate at a temperature higher than room temperature or in the presence of water.

21. A method for cleaning a textile or surface, comprising contacting the textile or surface with the composition of claim 1.

22. The method of claim 21, wherein the amadoriase oxidizes one or more substances in a stain on the textile or surface or in the composition to produce hydrogen peroxide in situ.

23. The method of claim 22, wherein the composition further comprises perhydrolase, and wherein the textile or surface is bleached and/or disinfected.


SEQUENCE LISTING (26 sequences)

SEQ ID NO: 1 - protein, 372 aa, Corynebakterium
MSSTATKHVAVIGGGILGVSTAVHLLRQGATVTLLTEQGLASEATGRSLSWLNSAGERSAPYHQLRIAGVDRYRTLFAADPSREWLQFGGGLMWNAAGESEVTKARHAYEKSIGYDSQLLAPEEIGSVTPGIDASAVPENAIFNPGEGWVSLPDLVNFLMEEFHALGGQLVLNAGKASVMVEGGRATGVETATGETYPADAVLVACGAATPAVVKPLGVEIPNGSPVSMLVVTKPVEHQVAAVLNTPRAAVRPNPGSTFAMDHDWYEGHITEHADGSFTIPDDVVQELADESSKLIAGNPELKPASWKIGYKPIPGDGEPVFGELGRVPGCFVAFTHSGATLGLIAGELLSGEILTGDKHPMFATFRPGRFS

SEQ ID NO: 2 - protein, 375 aa, Agrobacterium tumefaciens
MTDNTISSAVIIGAGIFGVSTGVQLARRGIQVTILNDGPPANGASGRSLSWLNSARMRSEPYHQLRMAGIDRYRTLAAENPDVEWLRFDGGLTWDSDDERNEIDAAYRHEVSLAYDAQRLSAGDVARVTPGIDAGAITPQGAIFNPGEGWVDLPTLIRVLLEEFFALGGVLVTDQGAARVMLEGGRAVGAETAEGYLHRADAVVLATGPAVPKMVGESGQIIGDGTPIALLVQTKPLAHPLRAVLNTPRVAVRPAPGGSFSLDADWAADEGVTVRADGTYEIDDTIVAELLVEAAKVMEGNPQLEVASIGVGGKPIPGDGEPVIGAIKAIPGYYVAFSHSGATLGLIVGELLAFEIVTGTEHQMLATFRPERFSS

SEQ ID NO: 3 - protein, 370 aa, Arthrobacter sp.
MSSTKHVAVIGGGILGVSTAVHLLRQGASVTLLSEQGLASEATGRSLSWLNSAGERSTPYHQLRVAGVDRYRTLFATDPSREWLQFGGGLMWNAAGQGEVTEARHAYEKSIAYDSKLLAPDEIESVTPGIDARAVPENAIFNPGESWVSLPELGDFLMEDFHALGGNLVLNAGKVSVTVDGGRAAGVETAAGDTYAADAVLVACGAATPAVVKALGVDIPNGSPVSMLVVTKPVEHNVTAVMNTPRAAVRPNPGNTFALDHDWYEERITEHADGSFTIPDEVVQELADESSKLIAGNPELKPASWKIGYKPIPGDGEPVFGELGQVPGCFVAFTHSGATLGLIAGELLSGEILTGEKHPMLATFRPGRFS

SEQ ID NO: 4 - protein, 441 aa, Aspergillus clavatus
MAPSPLSIESSILIIGAGTWGCSTALDIARRGYKHVTVLDPHPVPSPIAAGNDINKIMEHNELKDGEPDSRSIAFATFTRAALNAWKTDPVFQPFFHETGAIVSGHTPALLKHIQEDEIDPSETAFVQLETAEDFRGTMPAGVLTGDFPGWKGWWRKDGAGWIHAKKAMVSAFSEARRLGVTFVTGSPEGNVDSLAYEGGDVIGARTADGKLHRADYTILSAGAGSDMLLDFKKQLRPTAWTLCHIQMTPEEAARYRNLPVLFNIAKGFFMEPDADNHELKICDEHPGYCNFLPDPDRPGETRSIPFAKHQIPLEAEARARDFLRDTMPHLAERPLSFARICWDADTPDRAFLIDKHPEYPSLVVAVGGSGNGAMQMPTIGGFIADALEGSLQKDLKDVVRWRPETAVGRDWRATQNRFGGPGQIMDFQNLEEGQWTRIKV

SEQ ID NO: 5 - protein, 438 aa, Aspergillus fumigatus
MAVTKSSSLLIVGAGTWGTSTALHLARRGYTNVTVLDPYPVPSAISAGNDVNKVISSGQYSNNKDEIEVNEILAEEAFNGWKNDPLFKPYYHDTGLLMSACSQEGLDRLGVRVRPGEDPNLVELTRPEQFRKLAPEGVLQGDFPGWKGYFARSGAGWAHARNALVAAAREAQRMGVKFVTGTPQGRVVTLIFENNDVKGAVTGDGKIWRAERTFLCAGASAGQFLDFKNQLRPTAWTLVHIALKPEERALYKNIPVIFNIERGFFFEPDEERGEIKICDEHPGYTNMVQSADGTMMSIPFEKTQIPKEAETRVRALLKETMPQLADRPFSFARICWCADTANREFLIDRHPQYHSLVLGCGASGRGFKYLPSIGNLIVDAMEGKVPQKIHELIKWNPDIAANRNWRDTLGRFGGPNRVMDFHDVKEWTNVQYRDISKL

SEQ ID NO: 6 - protein, 445 aa, Aspergillus oryzae
MTSSKLTPTSSILIVGAGTWGCSTALHLARRGYKNVTVLDPHPVPSPIAAGNDINKIMEHREVKASETDPWSIAFSTCTRAALKGWKNDPVFQPYFHETGAIVSGHTASLIKHIQEHEIDSSDAEFIKLNTAEDFRKTMPPGILTGNFPGWKGWLNKTGAGWIHAKKAMFSAYTEAKRLGVTFITGSPEGDVVSLIYENGDVVGARTADGTVHRADHTILSAGAGSDRLLDFKKQLRPTAWTLCHIRMTPDEAKKYRNLPVLFNVAKGFFMEPDEDNHELKICDEHPGYCNFVPDPKHGGEVRSIPFAKHQIPLEAEARARDFLRDTMPHLADRPLSFARICWDADTVDRAFLIDRHPEYRSLLLAVGGSGNGAMQMPTIGGFIADALEGNLQKELKHALRWRPEIAAQRDWKDTQNRFGGPNKVMDFQKVGENEWTKIGDKSRL

SEQ ID NO: 7 - protein, 436 aa, Aspergillus oryzae
MTVAKSSSILIIGAGTWGASTALHLGRRGYTNVTVLDPYTVPSAISAGNDVNKIISSGQYSNKKDEIEVNEILAEEAFKGWTTDPLFKPYYHDTGVVMSACSSAGLDRLGIRVRPEEEPDVSEVTKPEHFRQLAPAVLKGNFPGWRGYHIRSNAGWAHARNALVAAIREAEKLGVKFVTGTQGRVITLIFENNDVKGAVTADGKIWRAEQTVLCAGANAAQFLDFKDQLRPTAWTLAHIRLKPEERALYKNLPVIFNIEKGFFFEPDEERGEIKICDEHPGYTNMVKSADGHLTSLPFEKTQIPKESEARVRALLSETMPQLADRPFSFARVCWCADTANREFIIDRHPEHPSLVLGCGASGRGFKYLPSIGNLIVDAIEDKVPEKVHKLTRWSPDIAVDRKWRDTLGRFGGPNRVMDFHDVKEWTNVQNKDTAKL

SEQ ID NO: 8 - protein, 437 aa, Aspergillus terreus
MPVTKSSSILIIGAGTWGCSTALHLARRGYTNVTVLDPYPVPSAISAGNDVNKIISSGQYSSKKDEVEVNEIIAEQAFNGWKNDPIFKPYYHDTGVVMSATTQEGLERLGVRVRPEDEPDVAELTRPEQFRQLAPGVLKGNFPGWRGYHIRSNAGWAHARNALVAAAREAQRLGVRFVAGSPQGRVITLIFENNDVKGAVTADGKIWRAEQTILCAGAAAGQFLDFKDQLRPTAWTLVHIQLKPEERAQYKNMPVVFNIEKGFFFEPDEERGEIKICDEHPGYTNMTTGADGRVRSIPFEKTQVPREAEMRVRKLLSETMPQLADRPFSFARICWCADTPNREFIIDRHPEYPSLVLGCGASGRGFKYLPSIGSIIADAMEDKTPAKIHKLIRWSPEIAINRNWGDRLGRFGGPNRVMDFNEVKEWTNVTQRDISKL

SEQ ID NO: 9 - protein, 438 aa, Aspergillus fumigatus
MAVTKSSSLLIVGAGTWGTSTALHLARRGYTNVTVLDPYPVPSAISAGNDVNKVISSGQYSNNKDEIEVNEILAEEAFNGWKNDPLFKPYYHDTGLLMSACSQEGLDRLGVRVRPGEDPNLVELTRPEQFRKLAPEGVLQGDFPGWKGYFARSGAGWAHARNALVAAAREAQRMGVKFVTGTPQGRVVTLIFENNDVKGAVTADGKIWRAERTFLCAGASAGQFLDFKNQLRPTAWTLVHIALKPEERALYKNIPVIFNIERGFFFEPDEERGEIKICDEHPGYTNMVQSADGTMMSIPFEKTQIPKEAETRVRALLKETMPQLADRPFSFARICWCADTANREFLIDRHPQYHSLVLGCGASGRGFKYLPSIGNLIVDAMEGKVPQKIHELIKWNPEIAANRNWRDTLGRFGGPNRVMDFHDVKEWTNVQYRDISKL

SEQ ID NO: 10 - protein, 445 aa, Aspergillus fumigatus
MAPSILSTESSIIVIGAGTWGCSTALHLARRGYKDVTVLDPHPVPSPIAAGNDINKIMEHSELKDGSSDPRSAAFSTFTRAALKAWKTDPVFQPYFHETGFIISGHTPALIDHIRKDEVEPSETNFVKLETAEDFRRTMPPGVLTGDFPGWKGWLHKSGAGWIHAKKAMISAFNEAKRLGVRFVTGSPEGNVVSLVYEDGDVVGARTADGRVHKAHRTILSAGAGSDSLLDFKKQLRPTAWTLCHIQMGPEEVKQYRNLPVLFNIAKGFFIEPDEDKLELKICDEHPGYCNFLPDPNRPGQEKSVPFAKHQIPLEAEARARDFLHDTMPHLADRPLSFARICWDADTPDRAFLIDRHPEHPSLLVAVGGSGNGAMQMPTIGGFIADALESKLQKEVKDIVRWRPETAVDRDWRATQNRFGGPDRIMDFQQVGEDQWTKIGESRGP

SEQ ID NO: 11 - protein, 445 aa, Aspergillus fumigatus
MAPSILSTESSIIVIGAGTWGCSTALHLARRGYKDVTVLDPHPVPSPIAAGNDINKIMEHSELKDGSSDPRSAAFSTFTRAALKAWKTDPVFQPYFHETGFIISGHTPALIDHIRKDEVEPSETNFVKLETAEDFRRTMPPGVLTGDFPGWKGWLHKSGAGWIHAKKAMISAFNEAKRLGVRFVTGSPEGNVVSLVYEDGDVVGARTADGRVHKAHRTILSAGAGSDSLLDFKKQLRPTAWTLCHIQMGPEEVKQYRNLPVLFNIAKGFFMEPDEDKHELKICDEHPGYCNFLPDPNRPGQEKSVPFAKHQIPLEAEARARDFLHDTMPHLADRPLSFARICWDADTPDRAFLIDRHPEHPSLLVAVGGSGNGAMQMPTIGGFIADALESKLQKEVKDIVRWRPETAVDRDWRATQNRFGGPDRIMDFQQVGEDQWTKIGESRGP

SEQ ID NO: 12 - protein, 438 aa, Aspergillus nidulans
MTPRANTKIIVVGGGGTMGSSTALHLLRAGYTPSNITVLDTCPIPSAQSAGYDLNKIMSIRLRNKPDLQLSLEALDMWKNDPLFKPFFHNVGMIDVSSTEEGIEGLRKKYQSLLDAGIGLEKTNFMLESEDEILAKAPHFTQEQIKGWKGLFCGDGGWLAAAKAINAIGQFLKEQGVKFGFGGAGTFKKPLFADAHEKTCIGVETVDGTKYYADKVVLAAGAWSSTLVDLEEQCVSKAWVFAHIQLTPAEAAAYKNTPVIYDGDYGFFFEPNENGIIKVCDEFPGFTHFKMHQPYGSPAPKPISVPRSHAKHPTDTYPHASEVTIKKAINRFLPRFNDKELFNRAMCWCTDTADANLLVCEHPRWKGFYLATGDSGHSFKLLPNIGKHVVELLEERLESVFKDAWRWRPGSGDALKSRRAAPAKDLADMPGWRNEAKM

SEQ ID NO: 13 - protein, 477 aa, Cryptococcus neoformans
MPPSRASTKVIVIGGGGTLGSSTALHLLRAGYTPSNITVLDTYPIPSAQSAGNDLNKIMGIRIRNPVDKQLSLEARDMWRNDEVFKPYFHNTGRLDCAHTPESIASLRKSYEAILKAGSGLEKTHHWLSTEDEILARAPLLDRKQIKGWKAIYSEDGGWLAAAKAINSIGQVLKEKGVTFGFGSAGSFKKPLFDEDGTKAIGIETVDGTQYFADKVVLAAGAWSPTLVDLEGQCCSKAWVYAHMQLTPEEAAEYKECPVVYNSELGFFFEPNEKGVIKVCDEFPGFTRFKQHQPYGASTTKHISFPRSHAKHPTDTIPDESDASIRRAISAFLPRFKEKELFNRALCWCTDTADANLLICEHPKWKNFILATGDSGHSFKILPNIGKHVVELIEGTLAEDLAESWRWRPGSGDPLISRRAAPARDLADLPGWNHDEPSDDDMDVKDVAVSLASVKIGENIGEKVVEDGARVGVKVLA

SEQ ID NO: 14 - protein, 371 aa, Gibberella zeae
MVLASSLTKQSQILIVGGGTWGCSTALHVTRRGYTNVTLITKSSIGHKIGDFVPLITAKDFRNTMPKGVLTGDFPGWKGFYKSKGSGWVHARKAMTAAFEESKRLGVKFITGSPKGEVQSLIFEGGDVKGVKTADGKEHRADRTILAVGASAERFLDFENQIRPTAWTIGHIQMTPEETQLYKNLPVLFNIEKGFFMEPDEDLHQLKICDEHPGYVNWMQKPGAKFPQSIPFAKHQIPLESEHRMRDFLRDIMPQLADRPLVHARLCWCADTYDRHFLITYHPRHPSLVVASGDRGIGYKHITSIGNFISDCMEGTLEERFAKVWRWRPEKFIEFWGKDPLERLGADHNIMDLPRSEDEGWTDISESVPIL

SEQ ID NO: 15 - protein, 446 aa, Neosartorya fischeri
MAPSNLTTESSILIIGAGTWGCSTALHLARRGYKDVTVLDPHPVPSPIAAGNDINKIMEHSELKDGSSDPRSAAFSTFTRAALKAWKTDPIFQPYFHETGFIISGHTPALIEHIRKDEVEPSETNFVKLETAEDFRRTMPPGVLTGDFPGWKGWWHKSGAGWVHAKKAMISAFNEAKRLGVKFVTSSPEGNVVSLVYEDGDVVGAKTADGRVHRAHRTILSAGAGSDSLLDFKKQLRPTAWTLCHIQMDPEEVKQYRNLPVLFNIAKGFFMEPDEDKHELKICDEHPGYCNFLSDPDRPGQEKSVPFAKHQIPLEAEARARDFLRDTMPHLAGRPLSFARICWDADTPDRAFLIDRHPEHPSLLVAVGGSGNGAMQIPTIGGFIADALESKLQKEVKDVVRWRPETAVDRDWRATQNRFGGPDRIMDFQQVGEDQWTKIGESRGQL

SEQ ID NO: 16 - protein, 412 aa, Schizosaccharomyces pombe
MVKNTSVIIVGAGVFGLSAALELTKRGGYTIKILDRAPPPVIDGSSVDANRIIRSDYADAVYCSMGIDALEEWRTNPLFKEQFYGSGLMFVGRDNVEYRDMSLENLTKMGVSAAKFQTTEELRKLFPKWIGELNDGEAGYANFSSGWANAEQSVKSVVNYLAHAGVSFISGPEGTVEELITEENVVKGVRTTTGAYMAEKLIFATGAWTASLLPNDHTRFLATGQPVAYIKLTPEEYIRFLTNPVYLDFDTGFYIFPPTPDGYLKFARHGYGFTRMQNLKSGKVESVPPKKPLVSPILPKEAELDLRRNLQRTYGEEISQRPFYKTRICYYTDTADAEFVFDYHPDYENLFVCTGGSGHGFKFFPILGKYSIGCMFRELEEPLLKKWRWKKENLEFAALDHSRAGPSRQELS

SEQ ID NO: 17 - protein, 437 aa, Coniochaeta sp.
MTSNRADTRVIVVGGGGTIGSSTALHLVRSGYAPANITVLDTFEIPSAQSAGHDLNKIMGIRLRNKVDLQMSLEARQMWKEDELFQPFFHNTGRMDCEHTPEGIEDLKKQYQALHDAGAGLEKTHAWLDNEDEILSKMPLLQRDQIQGWKAIWSQDGGWLAAAKAINAIGQFLKERGVKFGFGGAGSFKQPLFDDEGTTCIGVETADGTKYYADKVVLAAGAWSPTLVDLEDQCCSKAWVYAHIQLTPEEAAEYKGVPVVYNGEFGFFFEPNEFGVIKVCDEFPGFSRFKEHQPYGAPSPKHISVPRSHAKHPTDTYPDASEVSIKKAIATFLPRFQDKELFNRALCWCTDTADAALLMCEHPKWKNFILATGDSGHSFKILPNVGKHVVELIEGRLPEEMAYQWRWRPGGDALKSRRAAPPKDLADMPGWKHDPKL

SEQ ID NO: 18 - protein, 372 aa, Corynebacterium sp.
MSSTATKHVAVIGGGILGVSTAVHLLRQGATVTLLTEQGLASEATGRSLSWLNSAGERSTPYHQLRIAGVDRYRTLFAADPSREWLQFGGGLMWNAAGESEVTKARHAYEKSIGYDSQLLAPEEIGSVTPGIDASAVPENAIFNPGEGWVSLPDLVNFLMEEFHALGGQLVLNAGKASVMVEGGRATAVETATGETYPADAVLVACGAATPAVVKPLGVEIPNGSPVSMLVVTKPVEHQVAAVMNTPRAAVRPNPGNTFALDHDWYEGHITEHADGSFTIPDDVVQELADESSKLIAGNPELKPASWKIGYKPIPGDGEPVFGELGRVPGCFVAFTHSGATLGLIAGELLSGEILTGDKHPMFATFRPGRFS

SEQ ID NO: 19 - protein, 437 aa, Eupenicillium terrenum
MAHSRASTKVVVVGGGGTIGSSTALHLIRSGYTPSNITVLDVYKTPSLQSAGHDLNKIMGIRLRNGPDLQLSLESLDMWQNDELFKPFFHQVGMIDCSSSKEGIENLRRKYQTLLDAGIGLEKTNVWLESEDEILAKAPNFTREQVKGWKGLFCTDGGWLAAAKAINAIGIFLQDKGVKFGFGGAGTFQQPLFAADGKTCIGLETTDGTKYFADKVVLAAGAWSPTLVDLEDQCVSKAWVFAHIQLTPKEADAYKNVPVVYDGEYGFFFEPNEYGVIKVCDEFPGFSRFKLHQPYGAASPKMISVPRSHAKHPTDTYPDASEVTIRKAIARFLPEFKDKELFNRTMCWCTDTADANLLICEHPKWKNFILATGDSGHSFKLLPNIGKHVVELLEGSLSQEMAGAWRWRPGGDALRSRRGAPAKDLAEMPGWKHDAHL

SEQ ID NO: 20 - protein, 441 aa, Fusarium oxysporum
MASTLTKQSQILIVGGGTWGCSTALHLARRGYTNVTVLDVNRIPSPISAGHDVNKLAGRLSTADSKGDDEDSIWKALSYAAAQGWLHDPVFQPFCHNTGSVVAGSTPKSIKQLVEDEIGDDIDQYTPLNTAEDFRKTMPEGILTGNFPGWKGFYKPTGSGWVHARKAMKAAFEESERLGVKFITGSPEGKVESLIFEDGDVRGAKTADGKEHRADRTILSAGASAEFFLDFENQIQPTAWTLGHIQMTPEETKLYKNLPPLFNINQGFFMEPDEDLHQLKMCDEHPGYCNWVEKPGSKYPQSIPFAKHQVPTEAERRMKQFLKDIMPQLADRPLVHARICWCADTQDRMFLITYHPRHPSLVIASGDCGTGYKHITSIGKFISDCMEGTLEERFAKFWRWRPEKFTEFWGKDPLDRFGADDKIMDLPKSDVEGWTNIKNDI

SEQ ID NO: 21 - protein, 430 aa, Neosartorya fischeri
MAVTKSSSLLIVGAGTWGTSTALHLARRGYTNVTVLDPYPVPSAISAGNDVNKVISSGQYSNNKDEIEINEILAEEAFNGWKNDPLFKPYYHDTGLLMSACSQEGLDRLGVRVRPGEDPNLVELTRPEQFRKLAPEGVLQGEFPGWKGYFARSGAGWAHARNALVAAAREAQRMGVKFVTGTPQGRVVTLIFENNDVKGAVTADGKIWRAERTFLCAGASAGQFLDFKNQLRPTAWTLVHIALKPEERALYKNIPVIFNIERGFFFEPDEERGEIKICDEHPGYTNMVQSADGTMMSIPFEKTQIPKEAETRVRALLKETMPQLADRPFSFARICWCADTANREFLIDRHPQYHSLVLGCGASGRGNLIVDAMEGKVPQKIHELIKWNPEIAANRNWKDTLGRFGGPNRVMDFHDVKEWTNVQYRDISKL

SEQ ID NO: 22 - protein, 438 aa, Aspergillus clavatus
MAVIKSSSLLIVGAGTWGTSTALHLARRGYTNVTVLDPYPVPSAISAGNDVNKIISSGQYSNKKDEIEINEILAEEAFDGWKNDPLFKPHYHDTGLLMSACTNEGLDRLGIRVRPGEDPNIVEVTQPDEFRKLAPPGVLQGDFPGWKGYFTRSGAGWAHARDALVAAAREAQRLGVRFVTGTPQGRVITLIFENNDVKGAVTADGKVWRAERTFLCAGASAGQFLDFKDQLRPTAWTLVHIALEPEERALYKNIPVIFNIERGFFFEPDEERGEIKICDEHPGHTNMVHFANGTAMSIPFEKTQVPLEAEIRVRALLSETMPQLADRPFSFARICWCADTANREFLIDQHPQYQSLILGCGASGRGFKYLPSIGGLIVDTMEGKVPQKIREIIRWNPEIAANRDWRDTLGRFGGPNSVMDFHEVKDWTNVQYRNISSL

SEQ ID NO: 23 - protein, 441 aa, Ulocladium sp.
MAPNRANISVIVVGGGGTIGSSTALHLVRSGYTPSNITVLDTYPIPSAQSAGNDLNKIMGIRLRNKVDLQLSLEARQMWTEDDLFKEYFHKTGRLDCAHGEKGLADLKQAYQALLDANAGLEATTEWLDSEDKILEKMPLLNRDQIKGWKAVFSEDGGWLAAAKAINAIGRFLRDQGVKFGFGGAGSFKQPLLAEGVCVGVETVDGTRYYADKVVLAAGAWSPALVDLQDQCVSKAWVYAHIQLSPSEAAEYKNVPVVYNGDVGFFFEPDEYGVIKVCDEFPGFTRFKQHQPFGASAPKRISVPRSAAKHPTDTYPDASEVSIRKAIATFLPKFTEKEVFNRHLCWCTDTADAALLMCEHPEWKNFVLATGDSGHTFKLLPNIGKHVVELLEGTLADDLAHAWRWRPGTGDALKSRRAARAKDLADMPGWNHDGEAPRAKL

SEQ ID NO: 24 - protein, 372 aa, artificial
  synthetic construct WO 02/44387
MSSTATKHVAVIGGGILGVSTAVHLLRQGATVTLLTEQGLASEATGISLSWLNSAGERSAPYHQLRIAGVDRYRTLFAADPSREWLQFGGGLMWNAAGESEVTKARHAYEKSIGYDSQLLAPEEIGSVTPGIDASAVPENAIFNPGEGWVSLPDLVNFLMEEFHALGGQLVLNAGKASVMVEGGRATGVETATGETYPADAVLVACGAATPAVVKPLGVEIPNGSPVSMLVVTKPVEHQVAAVLNTPRAAVRPNPGSTFAMDHDWYEGHITEHADGSFTIPDDVVQELADESSKLIAGNPELKPASWKIGYKPIPGDGEPVFGELGRVPGCFVAFTHSGATLGLIAGELLSGEILTGDKHPMFATFRPGRFS

SEQ ID NO: 25 - protein, 437 aa, Penicillium janthinellum
MAHSRESTKIVIVGGGGTMGSSTALHLIRSGYTPSNITVLDVYPIPSLQSAGYDLNKIMSIRLRNGPDLQLSLEALDMWKNDPLFKPFFHNVGMLDCSSSQEGIASLRRKHQDLIDANIGLEKTNIWLESEDDILAKAPHFTREQIKGWKGLFCGDGGWLAAAKAINAIGTFLKSQGVKFGFGSAGTFKRPLFAPDGATCSGVETVDGTKYFADKVVLAAGAWSSTLVDLEDQCVSKAWVFAHIQLTPQESAQYKDVPVVYDGDYGFFFEPNEHGVIKVCDEFPGFSRFKLHQPYGATSPKLISVPRSHAKHPTDTYPDSSEETIRKAIARFMPRFKDKELFNRSMCWCTDTADANLLICEHPKWKNFILATGDSGHSFKVLPNIGKHVVELIEGRLPQDLAGAWRWRPGGDALKSKRSAPAKDLAEMPGWKHDAKL

SEQ ID NO: 26 - protein, 427 aa, Pichia sp.
MESIIIVGAGTFGLSTALQLARDGYKNIKCFDKFPVPSEIAAGNDSNKIFHYDYVAPLAKPNSKERLSLEALHLWKTDPVYKPYYHPVGFILAASSDAPLLHDKEYYEELQKNGLRNYRYISTPEEFREYLPILKGPLPNWRGYVLDGDNGWLHARDSLKSAYEECKRLGVEFVFGDDGEIVELLNENGKLTGIRARSGAIFSAQKYVLSSGANAVTLLNFQRQLEGKCFTLAHFKVTDEEAKAFKSLPVLFNAEKGFFFEADENNEIKICNEYPGFTHTNESGESIPLYRMEIPLESALEIRQYLKETMPQFADRPFTKTRICWCTDSPDMQLILCTHPEYTNLIVASGDSGNSFKIMPIIGKYVSKVVTKGDKGLDPEDKECWKWRPETWDKRGQVRWGGRYRVADLNEIEEWVSVENPTPHKLE